(19) United States
(12) Patent Application Publication
(10) Pub. No.: US 2025/0101028 A1
(43) Pub. Date: Mar. 27, 2025

(54) HETEROCYCLIC COMPOUNDS, COMPOSITIONS THEREOF, AND METHODS OF TREATMENT THEREWITH

(71) Applicant: BeiGene, Ltd., Grand Cayman (KY)

(72) Inventors: Chao YU, Beijing (CN); Jie CHEN, Beijing (CN); Hanzi SUN, Beijing (CN); Huaqing LIU, Beijing (CN); Ce WANG, Beijing (CN); Zhiwei WANG, Beijing (CN)

(73) Assignee: BeiGene, Ltd., Grand Cayman (KY)

(21) Appl. No.: 18/889,657

(22) Filed: Sep. 19, 2024

Related U.S. Application Data
(63) Continuation of application No. PCT/CN2023/083294, filed on Mar. 23, 2023.

(30) Foreign Application Priority Data
Mar. 24, 2022 (WO) PCT/CN2022/082722

(51) Int. Cl. C07D 487/04; A61K 31/517; A61K 31/519; A61K 31/537; A61K 31/5377; A61K 31/55; C07D 519/00

(52) U.S. Cl. CPC C07D 487/04; A61K 31/517; A61K 31/519; A61K 31/537; A61K 31/5377; A61K 31/55; C07D 519/00

(57) ABSTRACT

Provided herein are compounds having the following structure:

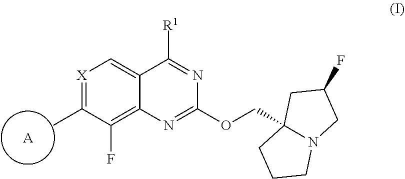

wherein the substituents are as defined herein, compositions comprising an effective amount of a compound, and methods for modulating activity of KRAS G12D and/or G12V.

### CROSS-REFERENCE TO RELATED APPLICATION(S)

This application is a continuation of International Patent Application No. PCT/CN2023/083294, filed Mar. 23, 2023, which claims priority from International Patent Application No. PCT/CN2022/082722, filed Mar. 24, 2022. The contents of these applications are incorporated herein by reference in their entirety.

### FIELD

Provided herein are heterocyclic compounds useful for treating cancer, a pharmaceutical composition comprising the compounds and, methods of using the compounds for treating cancer or a condition treatable or preventable by inhibition of KRAS G12V activity, comprising administering an effective amount of the compounds to a subject in need thereof.

### BACKGROUND

Ras is a family of proteins which are associated with cell membrane through their C-terminal membrane targeting region and well known as the molecular switch in intracellular signaling network (Cox A D, Der C J. Ras history: The saga continues. Small GTPases. 2010; 1(1):2-27). Ras proteins bind with either GTP or GDP and switch between “on” and “off” states. When Ras proteins bind with GDP, it is in the off (or inactive) state. And when Ras is switched on by certain growth promoting stimuli like growth factors, Ras proteins will be induced to exchange its bound GDP for a GTP and turn into on (or active) state (Malumbres M, Barbacid M. RAS oncogenes: the first 30 years. Nat Rev Cancer. 2003; 3(6):459-465). By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). Ras superfamily contains different subfamilies including Ras, Ral, Rap, Rheb, Rad, Rit and Miro (Wennerberg K, Rossman K L, Der C J. The Ras superfamily at a glance. J Cell Sci. 2005; 118(Pt 5):843-846). HRas, NRas and KRas are the most well studied proteins in Ras family since these proteins are the most common oncogenes in human cancers (O'Bryan J P. Pharmacological targeting of RAS: Recent success with direct inhibitors. Pharmacol Res. 2019; 139:503-511).

KRas is one of the most frequently mutated genes in human cancers. Based on data from Catalogue of Somatic Mutations (COSMIC) database, KRas mutation can be found in about 20% of human cancers, including pancreatic cancer, colorectal cancer, lung cancer, skin cancer etc. (O'Bryan J P. Pharmacological targeting of RAS: Recent success with direct inhibitors. Pharmacol Res. 2019; 139:503-511). The most common KRas mutations are found at position G12 and G13 by blocking the GTPase activating proteins (GAP) stimulated GTP hydrolysis activity of KRas (Wang W, Fang G, Rudolph J. Ras inhibition via direct Ras binding—is there a path forward?. Bioorg Med Chem Lett. 2012; 22(18):5766-5776). That results in the over activation of KRas protein and ultimately leads to uncontrolled cell proliferation and cancer.

Among different cancers, pancreatic cancer is considered as the most KRas-addicted cancer type. KRas mutation is found in 94.1% of pancreatic ductal adenocarcinoma (PDAC). G12D (41%) and G12V (34%) mutations of KRas are the two most predominant mutations in all the KRas mutated PDAC (Waters A M, Der C J. KRAS: The Critical Driver and Therapeutic Target for Pancreatic Cancer. Cold Spring Harb PerspectMed. 2018; 8(9):a031435). In vivo data generated by mouse models proves that the progression and maintenance of pancreatic cancer are highly rely on the constitutive activation of KRas downstream signaling (Siveke J T, Schmid R M. Chromosomal instability in mouse metastatic pancreatic cancer—it's Kras and Tp53 after all. Cancer Cell. 2005; 7(5):405-407). Which indicates that mutated KRas protein is a highly attractive drug target for pancreatic cancer and also other cancers with KRas mutation. Since WT KRas protein also plays critical role in the function of normal tissue and WT KRas function is demonstrated to be essential for adult hematopoiesis (Malumbres M, Barbacid M. RAS oncogenes: the first 30 years. Nat Rev Cancer. 2003; 3(6):459-465). It is highly deserved that a potential drug molecule can selectively inhibit mutated KRas protein in cancer cells and spare its WT companion in normal cells.

Thus, KRas G12D and G12V mutation is a highly attractive target for pancreatic cancer and other cancers with this mutation. As such, small-molecule therapeutic agents that are capable to selectively bind with KRas G12D and/or G12V and inhibit its function would be considered as an attractive strategy to target cancers with this mutation.

Citation or identification of any reference in this section is not to be construed as an admission that the reference is prior art to the present application.

### SUMMARY

Provided herein are compounds having the following formula (I):

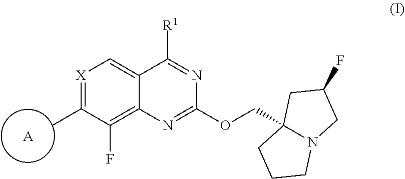

and pharmaceutically acceptable salts, tautomers, stereoisomers, enantiomers, atropisomers, isotopologues, and prodrugs thereof,
wherein:
ring A is

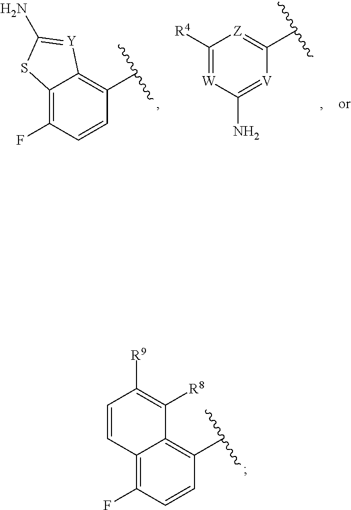

X is N, or C—R²;
Y is N, or C—CN;
Z is N, or C—R³;
W is N, or C—R⁵;
V is N, or C—R⁷;
R¹ is —NR^{1a}R^{1b};
R² is halogen, or unsubstituted or substituted alkyl;
R³ is halogen, or unsubstituted or substituted alkyl;
R⁴ is halogen, —NO₂, or unsubstituted or substituted alkyl;
R⁵ is hydrogen, halogen, or —CN;
R⁷ is hydrogen, halogen, or —CN;
each of R⁸, R⁹, and R¹⁰ is independently hydrogen, halogen, unsubstituted or substituted alkyl, or —OH;
R^{1a} and R^{1b} are each independently hydrogen, unsubstituted or substituted alkyl, unsubstituted or substituted cycloalkyl, unsubstituted or substituted cycloalkylalkyl, unsubstituted or substituted heterocyclyl, unsubstituted or substituted heterocyclylalkyl, provided that R^{1a} and R^{1b} are not both hydrogen; or
R^{1a} and R^{1b}, together with the nitrogen atom to which they are attached to, form an unsubstituted or substituted heterocyclyl.

In one embodiment, the compound having formula (I) is a compound, wherein ring A is

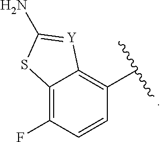

In one embodiment, Y is N. In one embodiment, Y is C—CN. In one embodiment, X is C—R². In one embodiment, R² is halogen, or alkyl which is unsubstituted or substituted by halogen; preferably Cl, or CF₃; more preferably CF₃. In one embodiment, R¹ is —NHR^{1a}. In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl.

In one embodiment, the compound having formula (I) is a compound, where ring A is

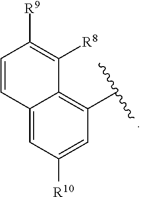

In one embodiment, X is C—R². In one embodiment, R² is halogen, or alkyl which is unsubstituted or substituted by halogen; preferably Cl, or CF₃; more preferably Cl. In one embodiment, R⁸ is methyl, ethyl, Cl, —CN, or —CCH; preferably —CCH. In one embodiment, R⁹ is hydrogen, or F. In one embodiment, R¹⁰ is —OH. In one embodiment, R¹ is —NHR^{1a}. In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl. In one embodiment, R^{1a} is cyclopropyl.

In one embodiment, the compound having formula (I) is a compound, where ring A is

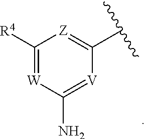

In one embodiment, X is C—R². In one embodiment, R² is halogen, or alkyl which is unsubstituted or substituted by halogen; preferably Cl, or CF₃; more preferably Cl.

In one embodiment, ring A is

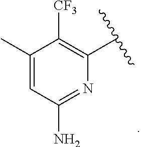 [SMILES: *c1nc(N)cc(C)c1C(F)(F)F]

In one embodiment, R^{1a} and R^{1b}, together with the nitrogen atom to which they are attached to, form an unsubstituted or substituted heterocyclyl.

In one embodiment, the compound having formula (I) is a compound, wherein ring A is

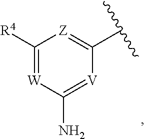

and R¹ is

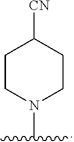 [SMILES: *N1CCC(C#N)CC1]

In one embodiment, ring A is

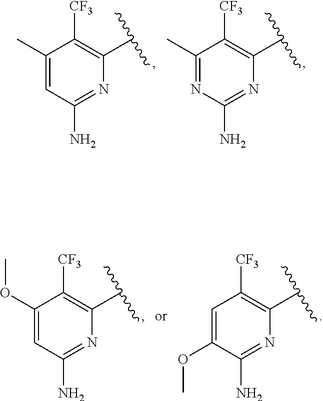

In one embodiment, the compound having formula (I) is a compound, wherein ring A is

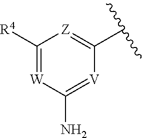

and X is N. In one embodiment, ring A is

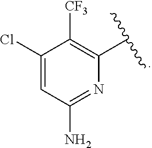 [SMILES: *c1nc(N)cc(Cl)c1C(F)(F)F]

R¹ is —NHR^{1a}. In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl.

In one embodiment, ring A is

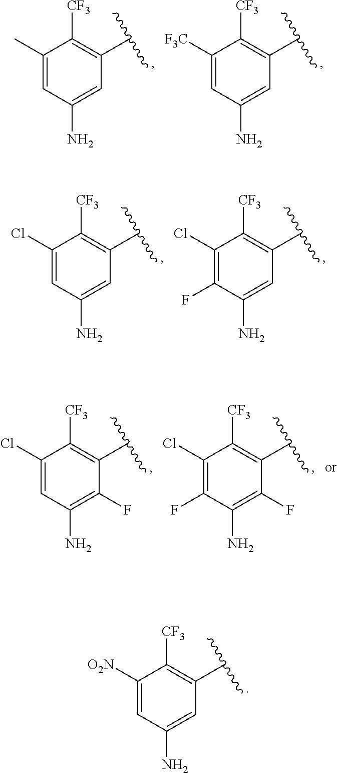

In one embodiment, ring A is NH₂.

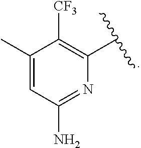 [SMILES: *c1nc(N)cc(C)c1C(F)(F)F]

In one embodiment, R^{1a} and R^{1b}, together with the nitrogen atom to which they are attached to, form an unsubstituted or substituted monocyclic heterocyclyl or an unsubstituted or substituted spiro heterocyclyl, said unsubstituted or substituted monocyclic heterocyclyl or the unsubstituted or substituted spiro heterocyclyl comprises zero, one, or two additional heteroatoms selected from oxygen, nitrogen, or optionally oxidized sulfur; preferably R¹ is unsubstituted or substituted heterocyclyl selected from unsubstituted or substituted azetidinyl, unsubstituted or substituted pyrrolidyl, unsubstituted or substituted piperidyl, unsubstituted or substituted morpholinyl, or unsubstituted or substituted.

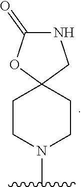 [SMILES: *N1CCC2(CC1)CNC(=O)O2]

In one embodiment, the compound having formula (I) is a compound, wherein R^{1a} and R^{1b} together with the nitrogen atom to which they are attached to, form an unsubstituted or substituted heterocyclyl; and the heterocyclyl is unsubstituted or substituted monocyclic heterocyclyl, unsubstituted or substituted bicyclic heterocyclyl, unsubstituted or substituted tricyclic heterocyclyl, unsubstituted or substituted quadracyclic heterocyclyl, or unsubstituted or substituted spirocyclic heterocyclyl.

In one embodiment, the compound is selected from Table 1, Table 2 and Table 3.

In one embodiment, provided herein is a method for inhibiting the activity of KRAS mutant protein in a cell, comprising contacting said cell with an effective amount of a compound provided herein, or a pharmaceutically acceptable salt, tautomer, isotopologue, stereoisomer, or prodrug thereof, optionally wherein the KRAS mutant protein is KRAS G12V mutant protein.

In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, or prodrug thereof, optionally wherein the cancer is mediated by KRAS mutation; preferably KRAS G12V mutation.

### DETAILED DESCRIPTION

### Definitions

As used herein, “KRAS gene” refers to a gene selected from the group consisting of: DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, and mutants thereof.

As used herein, “KRAS protein” refers to a protein or an isoform thereof expressed by a KRAS gene (Scolnick E M, Papageoege A G, Shih TY (1979), “Guanine nucleotide-binding activity for src protein of rat-derived murine sarcoma viruses,” Proc Natl Acad Sci USA. 76 (5): 5355-5559; Kranenburg O (November 2005) “The KRAS oncogene: past, present, and future,” Biochimica et Biophysica Acta (BBA)—Reviews on Cancer, 1756 (2): 81-2).

As used herein, “G12V mutation” refers to the mutation of the 12^{th} amino acid residue located in the G domain of KRAS protein from glycine to a valine.

As used herein, “KRAS G12V” or “G12V” refer to KRAS protein with G12V mutation.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to +0.2° 20 (or +0.2 degree 20) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, ortetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R) or —C(O)NH₂, wherein each R is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and Rare independently as defined above.

A “sulfonylamino” group is a radical of the formula: —NHSO₂(R^{#}) or —N(alkyl)SO₂(R^{#}), wherein each alkyl and Rare defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and Rare independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E.L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

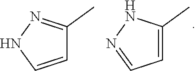

As readily understood by one skilled in the art, a wide variety of functional groups and other structures may exhibit tautomerism and all tautomers of compounds of formula (I) are within the scope of the present invention.

It should also be noted the compounds can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (¹⁴C), or may be isotopically enriched, such as with deuterium (H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of KRAS G12V.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of KRAS G12V.

The term “effective amount” in connection with a compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### Compounds

Aspect 1: Provided herein are compounds having the following formula (I):

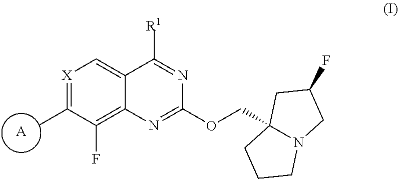

and pharmaceutically acceptable salts, tautomers, stereoisomers, enantiomers, atropisomers, isotopologues, and prodrugs thereof,
wherein:
ring A is

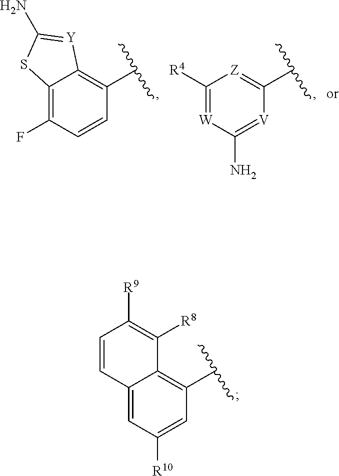

X is N, or C—R²;
Y is N, or C—CN;
Z is N, or C—R³;
W is N, or C—R⁵;
V is N, or C—R⁷;
R¹ is —NR^{1a}R^{1b};
R² is halogen, or unsubstituted or substituted alkyl;
R³ is halogen, or unsubstituted or substituted alkyl;
R⁴ is halogen, —NO₂, or unsubstituted or substituted alkyl;
R⁵ is hydrogen, halogen, or —CN;
R⁷ is hydrogen, halogen, or —CN;
each of R⁸, R⁹, and R¹⁰ is independently hydrogen, halogen, unsubstituted or substituted alkyl, or —OH;
R^{1a} and R^{1b} are each independently hydrogen, unsubstituted or substituted alkyl, unsubstituted or substituted cycloalkyl, unsubstituted or substituted cycloalkylalkyl, unsubstituted or substituted heterocyclyl, unsubstituted or substituted heterocyclylalkyl, provided that R^{1a} and R^{1b} are not both hydrogen; or
R^{1a} and R^{1b}, together with the nitrogen atom to which they are attached to, form an unsubstituted or substituted heterocyclyl.

Aspect 2: In one embodiment, the compound having formula (I) is a compound, wherein ring A is

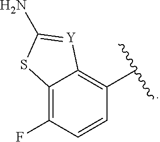

In one embodiment, Y is N.

In one embodiment, Y is C—CN.

In one embodiment, X is C—R². In one embodiment, R² is halogen, or alkyl which is unsubstituted or substituted by halogen; preferably Cl, or CF₃; more preferably CF₃.

In one embodiment, R⁷ is —NHR^{1a}. In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl. In one embodiment, R^{1a} is cycloalkyl, or heterocyclyl, wherein at least one ring of R^{1a} is cyclopropyl; and R^{1a} is optionally substituted by one or more C₁₋₄ alkyl, C₁₋₄ alkoxyl, halogen, —CN, —OH or acetyl, wherein each of the C₁₋₄ alkyl, and the C₁₋₄ alkoxyl, is independently optionally substituted by one or more halogen, —CN, —OH, or contains a carbon-carbon triple bond. In one embodiment, R^{1a} is cyclopropyl, cyclobutyl, cyclopentyl, cyclohexyl, spiro[3.3]heptan-2-yl, 2-oxaspiro[3.3]heptan-6-yl, bicyclo[1.1.1]pentan-1-yl, 3-oxabicyclo[3.1.0]hexan-6-yl, 1-(3-azabicyclo[3.1.0]hexan-3-yl)ethan-1-one-6-yl, or

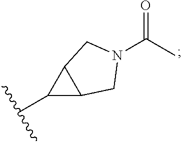 [SMILES: *C1C2CN(C(C)=O)CC12]

preferably cyclopropyl; R^{1a} is optionally substituted by one or more C₁₋₄ alkyl, halogen, —CN, —OH or C₁₋₄ alkoxyl; and each of the C₁₋₄ alkyl, and the C₁₋₄ alkoxyl, is independently optionally substituted by one or more halogen, —CN, —OH, or contains a carbon-carbon triple bond. In one embodiment, R^{1a} is

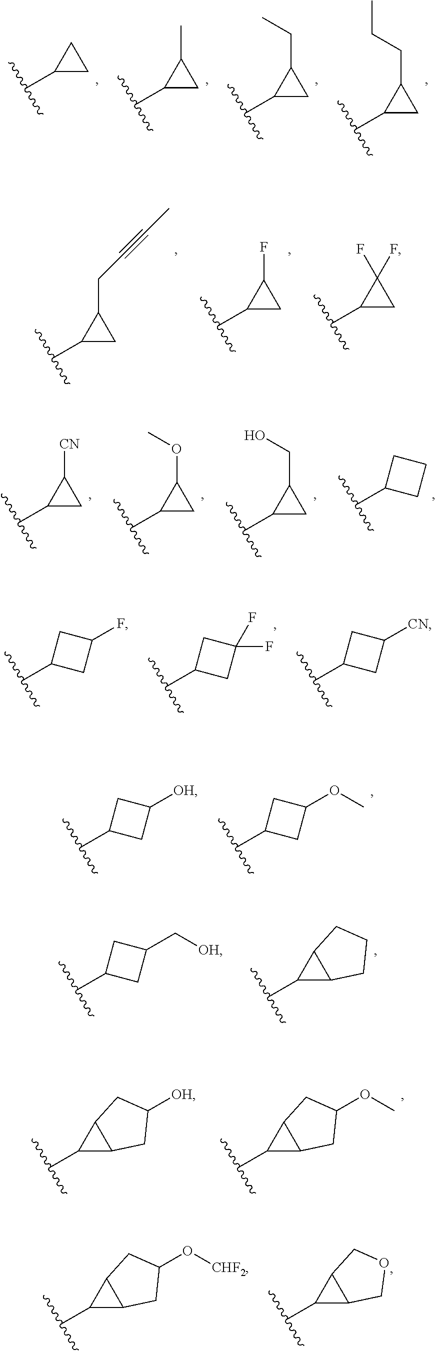

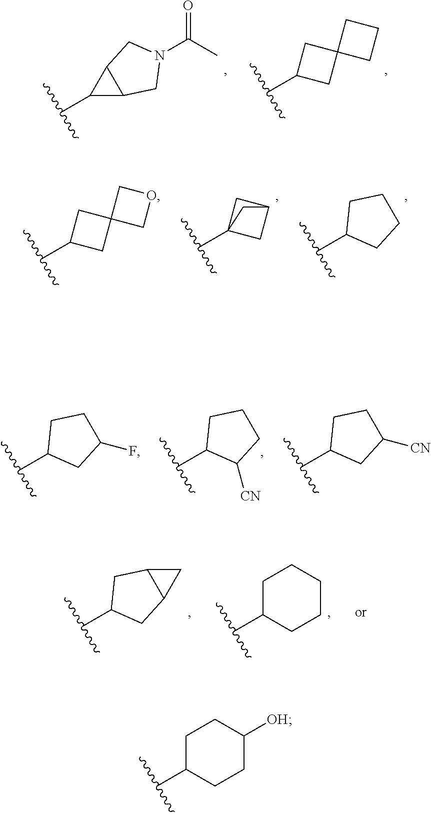

preferably

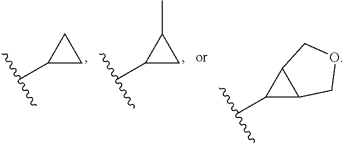

Aspect 3: In one embodiment, the compound having formula (I) is a compound, where ring A is

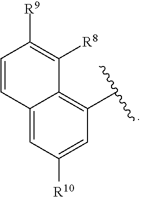

In one embodiment, X is C—R². In one embodiment, R² is halogen, or alkyl which is unsubstituted or substituted by halogen; preferably Cl, or CF₃; more preferably Cl.

In one embodiment, R⁸ is methyl, ethyl, Cl, —CN, or —CCH; preferably —CCH.

In one embodiment, R⁹ is hydrogen, or F.

In one embodiment, R¹⁰ is —OH.

In one embodiment, R¹ is —NHR^{1a}. In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl. In one embodiment, R^{1a} is cyclopropyl.

In one embodiment, R¹ is —NR^{1a}R^{1b}, wherein R^{1a} and R^{1b}, together with the nitrogen atom to which they are attached to, form an unsubstituted or substituted monocyclic heterocyclyl or an unsubstituted or substituted spiro heterocyclyl. In some embodiment, the unsubstituted or substituted monocyclic heterocyclyl or the unsubstituted or substituted spiro heterocyclyl comprises one or two additional heteroatoms selected from oxygen, nitrogen, or optionally oxidized sulfur. In one embodiment, R^{1a} and R^{1b}, together with the nitrogen atom to which they are attached to, form an unsubstituted or substituted heterocyclyl selected from an unsubstituted or substituted azetidinyl, unsubstituted or substituted pyrrolidyl, unsubstituted or substituted piperidyl, unsubstituted or substituted azepanyl, unsubstituted or substituted

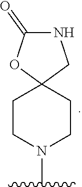 [SMILES: *N1CCC2(CC1)CNC(=O)O2]

unsubstituted or substituted

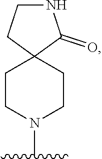 [SMILES: *N1CCC2(CCNC2=O)CC1]

unsubstituted or substituted

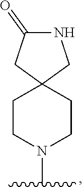 [SMILES: *N1CCC2(CC1)CNC(=O)C2]

unsubstituted or substituted

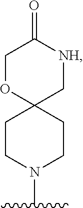 [SMILES: *N1CCC2(CC1)CNC(=O)CO2]

or unsubstituted or substituted

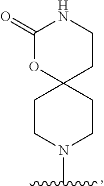 [SMILES: *N1CCC2(CCNC(=O)O2)CC1]

In one embodiment, the heterocyclyl is optionally substituted by one or more C₁₋₄ alkyl, halogen, —CN, —OH, C₁₋₄ alkoxyl, cycloalkylalkyl or methylsulfonyl, wherein each of the C₁₋₄ alkyl, and the C₁₋₄ alkoxyl, is independently optionally substituted by one or more halogen, —CN, —OH, or C₁₋₄ alkoxyl.

In one embodiment, R¹ is azetidine-1-yl,

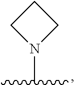 [SMILES: *N1CCC1]

2-methyl-azetidin-1-yl,

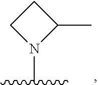 [SMILES: *N1CCC1C]

3-methyl-azetidin-1-yl,

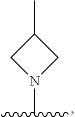 [SMILES: *N1CC(C)C1]

2,2-dimethyl-azetidin-1-yl,

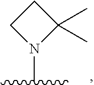 [SMILES: *N1CCC1(C)C]

3-fluoro-azetidin-1-yl,

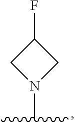 [SMILES: *N1CC(F)C1]

3,3-difluoro-azetidin-1-yl,

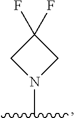 [SMILES: *N1CC(F)(F)C1]

3-cyano-azetidin-1-yl

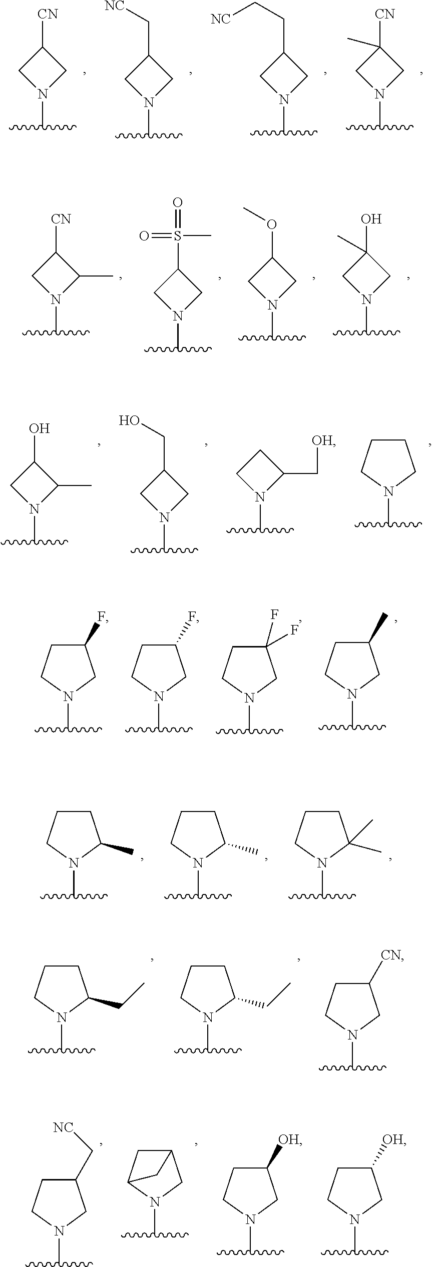

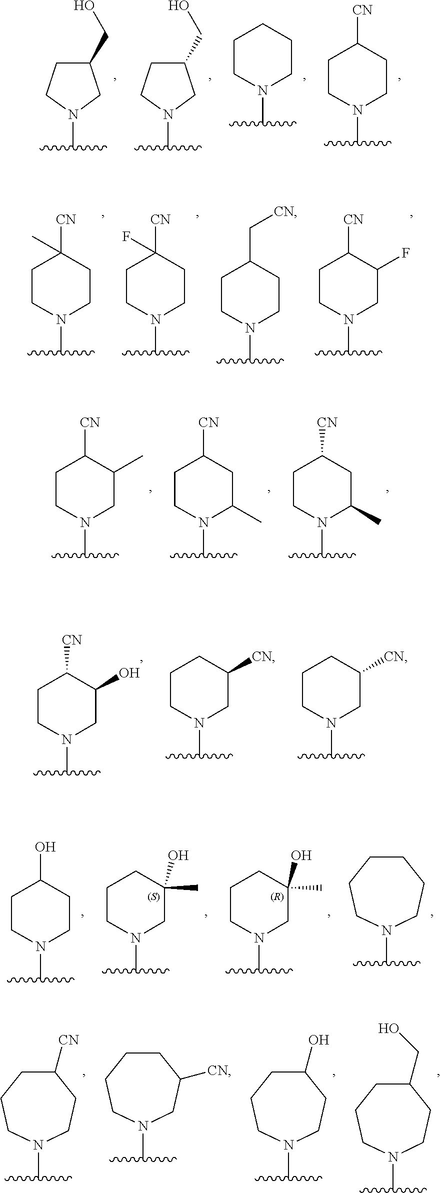

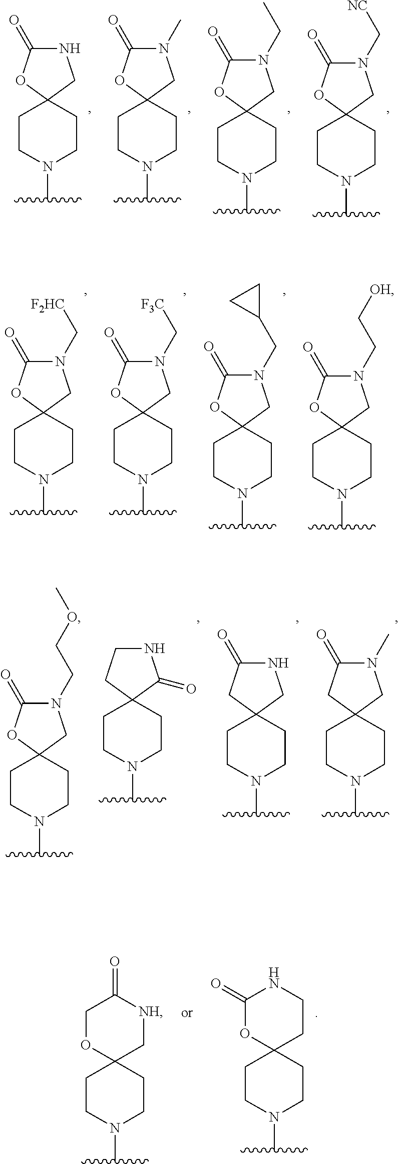

Aspect 4: In one embodiment, the compound having formula (I) is a compound, where ring A is

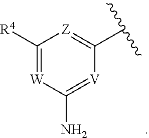

In one embodiment, X is C—R².

In one embodiment, R² is halogen, or alkyl which is unsubstituted or substituted by halogen; preferably Cl, or CF₃; more preferably Cl.

In one embodiment, ring A is

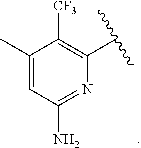 [SMILES: *c1nc(N)cc(C)c1C(F)(F)F]

In one embodiment, R^{1a} and R^{1b}, together with the nitrogen atom to which they are attached to, form an unsubstituted or substituted heterocyclyl selected from unsubstituted or substituted azetidinyl, unsubstituted or substituted pyrrolidyl, unsubstituted or substituted piperidyl, unsubstituted or substituted morpholinyl, unsubstituted or substituted

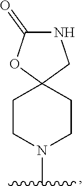 [SMILES: *N1CCC2(CC1)CNC(=O)O2]

In one embodiment, the heterocyclyl is optionally substituted by one or more C₁₋₄ alkyl, halogen, —CN, or —OH.

In one embodiment, R¹ is

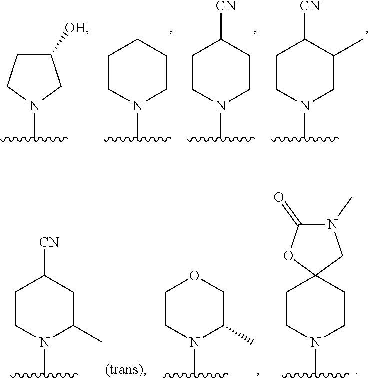

Aspect 5: In one embodiment, the compound having formula (I) is a compound, wherein ring A is

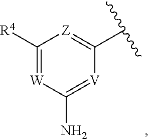

and R¹ is

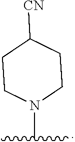 [SMILES: *N1CCC(C#N)CC1]

In one embodiment, ring A is

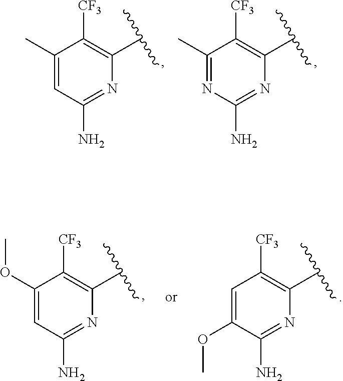

Aspect 6: In one embodiment, the compound having formula (I) is a compound, wherein ring A is

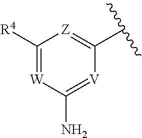

and X is N.

In one embodiment, ring A is

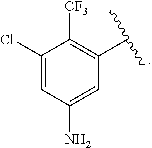 [SMILES: *c1cc(N)cc(Cl)c1C(F)(F)F]

In one embodiment, R¹ is —NHR^{1a}.

In one embodiment, R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl.

In one embodiment, R^{1a} is cycloalkyl, or heterocyclyl, wherein at least one ring of R^{1a} is cyclopropyl; and R^{1a} is optionally substituted by one or more C₁₋₄ alkyl.

In one embodiment, R^{1a} is cyclopropyl, cyclobutyl, bicyclo[1.1.1]pentan-1-yl, or 3-oxabicyclo[3.1.0]hexan-6-yl; preferably cyclopropyl; and R^{1a} is optionally substituted by one or more C₁₋₄ alkyl.

In one embodiment, R^{1a} is

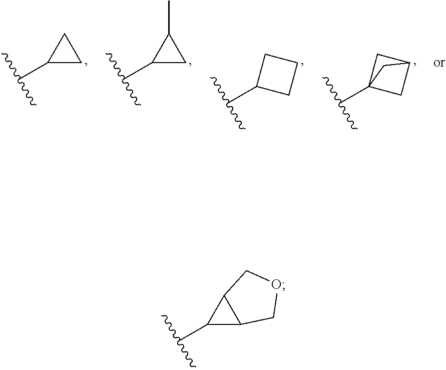

preferably

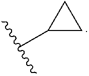 [SMILES: *C1CC1]

In one embodiment, R^{1a} and R^{1b}, together with the nitrogen atom to which they are attached to, form an unsubstituted or substituted heterocyclyl, wherein at least one ring of the heterocyclyl is piperidyl, or morpholinyl.

In one embodiment, R¹ is optionally substituted by one or more C₁₋₄ alkyl, halogen, —CN, or —OH, wherein each of the C₁₋₄ alkyl is independently optionally substituted by one or more halogen, —CN, or —OH.

In one embodiment, R¹ is

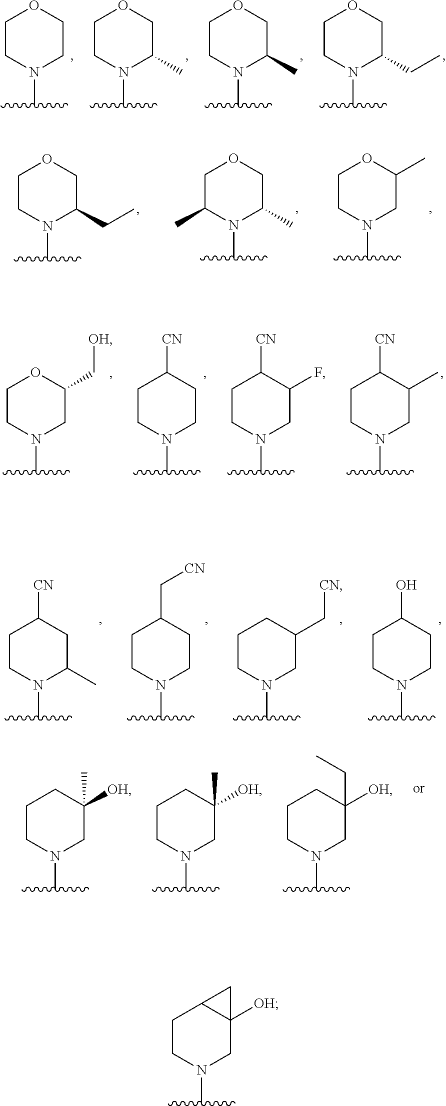

preferably

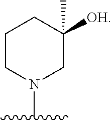 [SMILES: *N1CCC[C@@](C)(O)C1]

In one embodiment, R¹ is

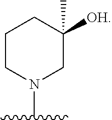 [SMILES: *N1CCC[C@@](C)(O)C1]

In one embodiment, ring A is

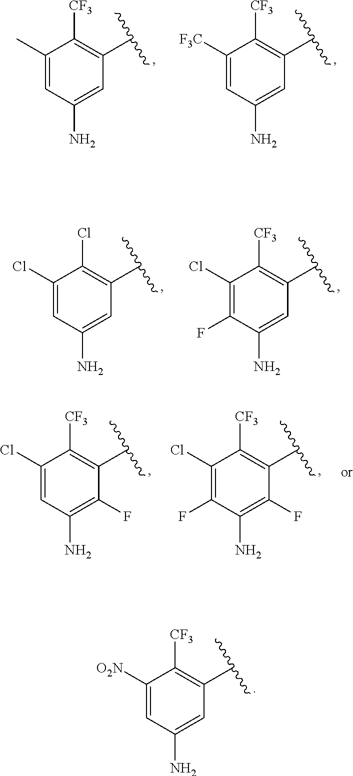

Aspect 7: In one embodiment, the compound having formula (I) is a compound, wherein R^{1a} and R^{1b}, together with the nitrogen atom to which they are attached to, form an unsubstituted or substituted heterocyclyl; and the heterocyclyl is unsubstituted or substituted monocyclic heterocyclyl, unsubstituted or substituted bicyclic heterocyclyl, unsubstituted or substituted tricyclic heterocyclyl, unsubstituted or substituted quadracyclic heterocyclyl, or unsubstituted or substituted spirocyclic heterocyclyl.

In one embodiment, R¹ is not unsubstituted or substituted piperazinyl.

In one embodiment, R¹ is not unsubstituted or substituted

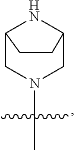 [SMILES: *N1CC2CCC(C1)N2]

unsubstituted or substituted

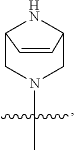 [SMILES: *N1CC2C=CC(C1)N2]

unsubstituted or substituted

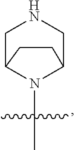 [SMILES: *N1C2CCC1CNC2]

unsubstituted or substituted

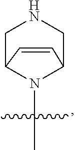 [SMILES: *N1C2C=CC1CNC2]

unsubstituted or substituted

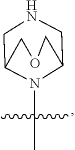 [SMILES: *N1C2CNCC1COC2]

unsubstituted or substituted

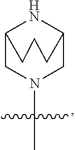 [SMILES: *N1CC2CCCC(C1)N2]

or unsubstituted or substituted

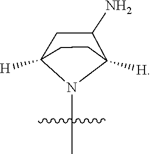

In one embodiment, R⁷ is not unsubstituted or substituted 3,8-diazabicyclo[3.2.1]octan-3-yl, or unsubstituted or substituted

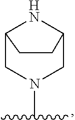 [SMILES: *N1CC2CCC(C1)N2]

Aspect 8: In one embodiment, the compound is selected from Table 1, Table 2 and Table 3.

Aspect 9: In one embodiment, provided herein is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Aspect 10: In one embodiment, provided herein is a method for inhibiting the activity of KRAS mutant protein in a cell, comprising contacting said cell with an effective amount of a compound provided herein, or a pharmaceutically acceptable salt, tautomer, isotopologue, stereoisomer, or prodrug thereof, optionally wherein the KRAS mutant protein is KRAS G12D and/or G12V mutant protein.

Aspect 11: In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, or prodrug thereof, optionally wherein the cancer is mediated by KRAS mutation; preferably KRAS G12D and/or G12V mutation.

Aspect 12: Provided here is a method of modulating activity of KRAS G12D and/or G12V, comprising contacting said cell with an effective amount of a compound provided herein, or a pharmaceutically acceptable salt, tautomer, isotopologue, stereoisomer, or prodrug thereof.

Aspect 13: Provided here is a method for the treatment or prevention of a cancer, the methods comprising administering to a subject in need thereof an effective amount of a compound provided herein.

Aspect 14: Provided herein is a kit for treating cancer, the kit comprising (a) a pharmaceutical composition comprising a compound provided herein; and (b) instructions for administration of an effective amount of the pharmaceutical composition comprising the KRAS G12D and/or G12V inhibitor provided herein to treat cancer in an individual.

The present embodiments can be understood more fully by reference to the detailed description and examples, which are intended to exemplify non-limiting embodiments.

### Methods for Making Compounds

The Compounds can be made using conventional organic syntheses and commercially available starting materials. By way of example and not limitation, Compounds of formula (I) can be prepared as outlined in Schemes 1-3 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Examplary protecting groups are described in “Protective Groups in Organic Synthesis”, 4^{th} Edition, P. G. M. Wuts; T. W. Greene, John Wiley, 2007, and references cited therein.

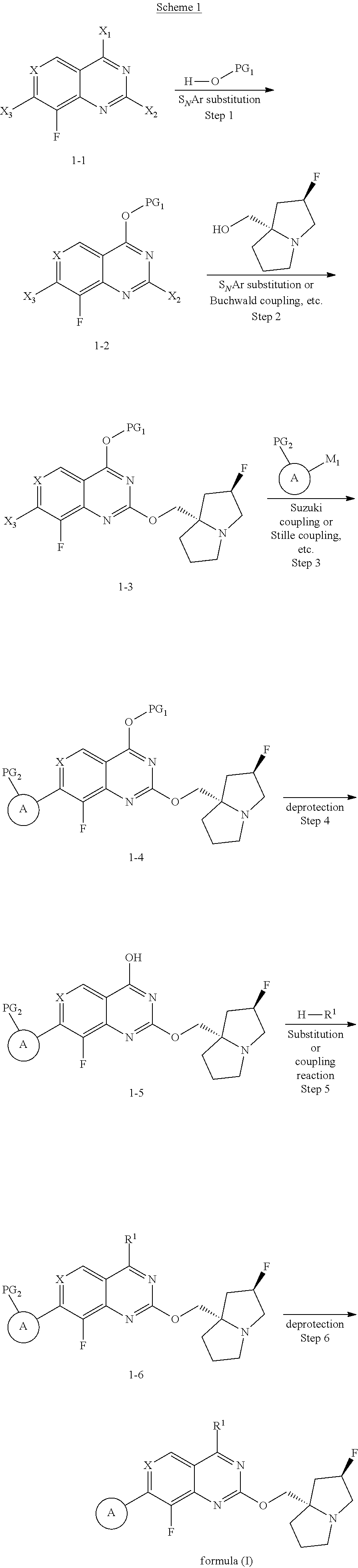

As shown in Scheme 1, in some embodiments, provided herein are methods for preparing the compounds defined as formula (I). Halogen substituted compound 1-1 (X₁, X₂, X₃ are halogen) is converted into compound 1-2 under basic conditions (e.g., NaH, THF); then compound 1-2 is converted to compound 1-3 under basic conditions (e.g., Cs₂CO₃, 1,4-dioxane) or Buchwald coupling conditions (e.g., RuPhos Pd catalyst, Cs₂CO₃, 1,4-dioxane); compound 1-3 further undergoes metal catalyzed cross-coupling reaction such as Suzuki or Stille coupling (e.g. Pd(dtbpf)Cl₂, K₃PO₄, 1,4-dioxane, water for Suzuki coupling) with A-M₁ where A may or may not contain protecting groups (PG₂) to obtain compound 1-4, wherein M₁ can be boronic acid, boronic ester, a metal (such as Zn), tributyltin, etc.; compound 1-5 is prepared by deprotecting compound 1-4 (e.g., 10% Pd/C, H₂, EtOAc to deprotect benzyl when PG₁ is Bn); compound 1-5 is converted to 1-6 under substitution or coupling reaction conditions (e.g., BOP or PyBOP, DIPEA, MeCN); compound 1-6 is then deprotected (e.g., TFA and DCM to deprotect Boc group when PG₁ is Boc) to yield the compound defined as formula (I).

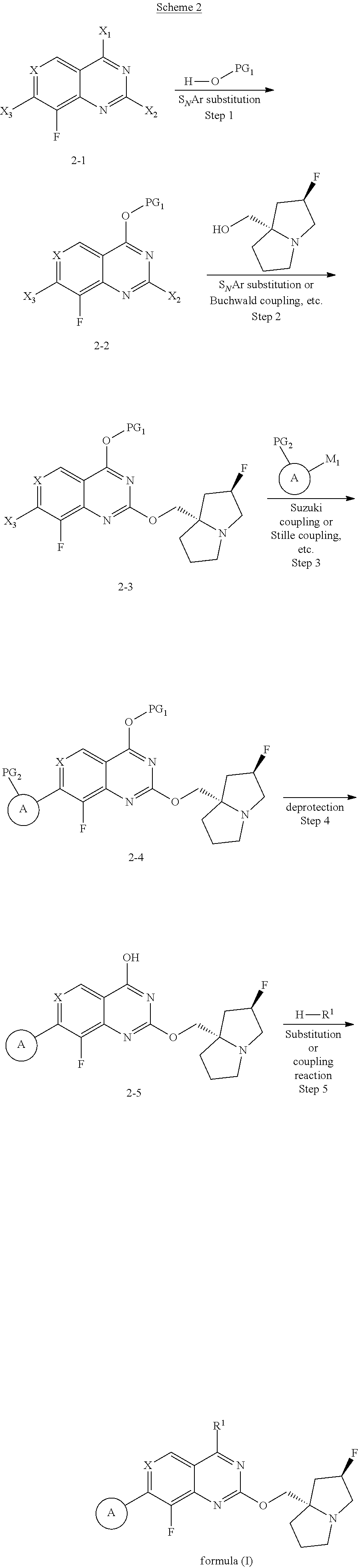

As shown in Scheme 2, in some embodiments, provided herein are methods for preparing the compounds defined as formula (I). Halogen substituted compound 2-1 (X₁, X₂, X₃ are halogen) is converted into compound 2-2 under basic conditions (e.g., NaH, THF); then compound 2-2 is converted to compound 2-3 under basic conditions (e.g., Cs₂CO₃, 1,4-dioxane) or Buchwald coupling conditions (e.g., RuPhos Pd catalyst, Cs₂CO₃, 1,4-dioxane); compound 2-3 further undergoes metal catalyzed cross-coupling reaction such as Suzuki or Stille coupling (e.g., Pd(dtbpf)Cl₂, K₃PO₄, 1,4-dioxane, water for Suzuki coupling) with A-M₁ where A may or may not contain protecting groups (PG₂) to obtain compound 2-4, wherein M₁ can be boronic acid, boronic ester, a metal (such as Zn), tributyltin, etc.; compound 2-5 is prepared by deprotecting compound 2-4 (e.g., TFA to deprotect benzyl and Boc when PG₁ is Bn and PG₂ is Boc); compound 2-5 is converted to the compound defined as formula (I) under substitution or coupling reaction conditions (e.g., BOP or PyBOP, DIPEA, MeCN).

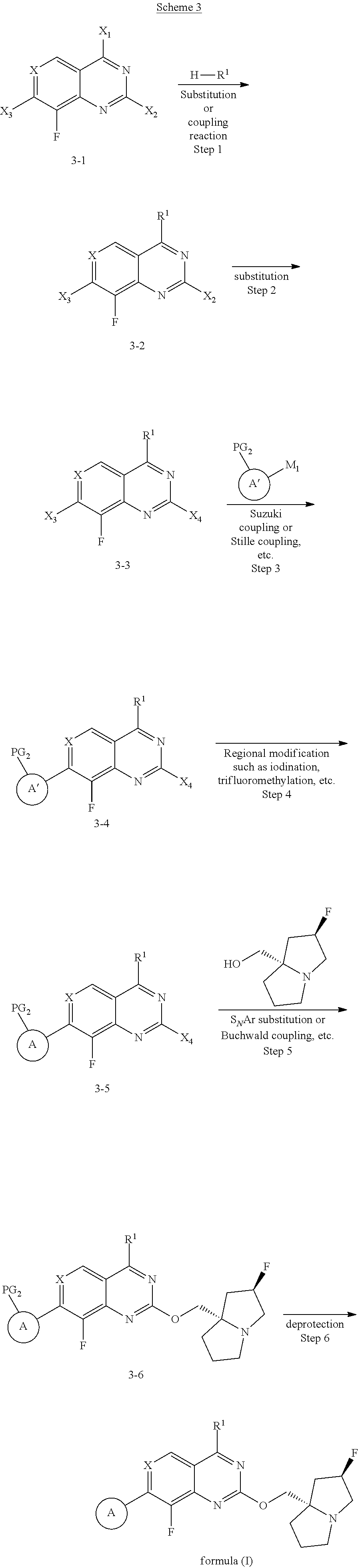

As shown in Scheme 3, in some embodiments, provided herein are methods for preparing the compounds defined as formula (I). Halogen substituted compound 3-1 (X₁, X₂, X₃ are halogen) is converted into compound 3-2 under substitution or coupling reaction conditions (e.g., DIPEA, DCM); then compound 3-2 is converted to compound 3-3 under substitution conditions (e.g., KF, DMSO where X₄ is F); compound 3-3 further undergoes metal catalyzed cross-coupling reaction such as Suzuki or Stille coupling (e.g. Pd(dppf)Cl₂, K₃PO₄, THF, water for Suzuki coupling) with A-M₁ where A may or may not contain protecting groups (PG₂) to obtain compound 3-4, wherein M₁ can be boronic acid, boronic ester, a metal (such as Zn), tributyltin, etc.; compound 3-5 is prepared by modifying compound 3-4 (e.g., I₂, Ag₂SO₄, and DMF for iodination; e.g., CuI, methyl 2,2-difluoro-2-(fluorosulfonyl)acetate, NMP for trifluoromethylation); compound 3-5 is converted to compound 3-6 under basic conditions (e.g., Cs₂CO₃, 1,4-dioxane) or Buchwald coupling conditions (e.g., RuPhos Pd catalyst, Cs₂CO₃, 1,4-dioxane); compound 3-6 is converted to the compound defined as formula (I) under deprotecting conditions (e.g., TFA to deprotect PMB group when PG₂ is PMB).

The present embodiments can be understood more fully by reference to the detailed description and examples, which are intended to exemplify non-limiting embodiments.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz with TMS (tetramethylsilane) as the internal standard. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 19.1.

In the following examples, the following abbreviations are used:

| | |
|---|---|
| AcOH | Acetic acid |
| Aq. | Aqueous |
| BINAP | 2,2′-bis(diphenylphosphino)-1,1′-binaphthalene |
| Brine | Saturated aqueous sodium chloride solution |
| Bn | Benzyl |
| BnBr | Benzyl Bromide |
| Boc | Tert-butoxycarbonyl |
| BOP | Benzotriazol-l-yl-oxy-tris- (dimethylamino)phosphonium |
| | hexa-fluorophosphate |
| CH₂Cl₂ or DCM | Dichloromethane |
| CAN | Cerium(IV) ammonium nitrate (cericammonium |
| | nitrate) |
| Cs₂CO₃ | Cesium carbonate |
| DAST | Diethylaminosulfur trifluoride |
| DCM | Dichloromethane |
| DMF | N,N-Dimethylformamide |
| Dppf | 1,1′-bis(diphenylphosphino)ferrocene |
| DBU | 1,8-diazabicyclo[5.4.0]undec-7-ene |
| DHP | 3,4-Dihydro-2H-pyran |
| DIEA or DIPEA | N,N-diisopropylethylamine |
| DMAP | 4-N,N-dimethylaminopyridine |
| DMB | (2,4-dimethoxyphenyl)methanamine |
| Dess-Martin/DMP | Dess-Martin Periodinane |
| DMF | N,N-dimethylformamide |
| DMF-DMA | N,N-Dimethylformamide dimethyl acetal purum |
| DMSO | Dimethyl sulfoxide |
| DMEDA | Dimethyl Ethylene Diamine |
| EDCI | 1-Ethyl-3-(3-dimethylaminopropyl)carbodiimide hydrochloride |
| EtOAc or EA | Ethyl acetate |
| EtOH | Ethanol |
| Et₃SiH | Triethyl silhydride |
| Et₂O or ether | Diethyl ether |
| g | Grams |
| h or hr | Hour |
| HATU | O-(7-Azabenzotriazol-1-yl)-N,N,N′,N′-tetramethyluronium |
| | hexafluorophosphate |
| Hex | Hexane |
| HCl | Hydrochloric acid |
| HMDS | Hexamethyldisilazane |
| HOBT | 1-Hydroxybenzotriazole |
| HPLC | High-performance liquid chromatography |
| IBX | 2-Iodylbenzoic acid |
| i-PrOH | Isopropyl alcohol |
| LCMS | Liquid chromatography-mass spectrometry |
| LDA | Lithium diisopropylamide |
| LiHMDS | Lithium Bis(trimethylsilyl)amide |
| K₂OsO₄•H₂O | Potassium osmate(VI) dihydrate |
| K₃PO₄ | Tripotassium phosphate |
| mg | Milligrams |
| mL | Milliliters |
| mmol | Millimole |
| MeCN | Acetonitrile |
| MeOH | Methanol |
| Min | Minutes |
| ms or MS | Mass spectrum |
| m-CPBA | 2-chloranylbenzenecarboperoxoic acid |
| MPLC | Medium Pressure Liquid Chromatography |
| Na₂SO₄ | Sodium sulfate |
| NaBH(OAc)₃/STAB | Sodium triacetyl borohydride |
| NaH | Sodium hydride |
| NaHMDS | Sodium bis(trimethylsilyl)amide |
| NBS | N-Bromosuccinimide |
| NCS | N-Chlorosuccinimide |
| NMO | 4-Methylmorpholine N-oxide |
| NMP | N-Methyl Pyrrolidone |
| PE | petroleum ether |
| PMB | (4-methoxyphenyl)methanamine |
| POCl₃ | phosphorous oxychloride |
| PyBOP | Benzotriazol-1-yl-oxytripyrrolidinophosphonium |
| | hexafluorophosphate |
| PddppfCl₂ | [1,1′-Bis(diphenylphosphino)ferrocene]dichloropalladium(II) |
| Pd(dtbpf)Cl₂ | [1,1′-Bis(di-tert- |
| | butylphosphino)ferrocene]dichloropalladium(II) |
| Pd₂(dba)₃ | Tris(dibenzylideneacetone)dipalladium |
| Prep | Preparative |
| PTSA | 4-Methylbenzenesulfonic acid |
| Rt or rt | Room temperature |
| RuPhos | 2-Dicyclohexylphosphino-2′,6′-diisopropoxybiphenyl |
| sat. | Saturated |
| SEMCl | (2-(Chloromethoxy)ethyl)trimethylsilane |
| TBSCl | tert-Butyldimethylsilyl chloride |
| TEA/ Et3N | triethylamine |
| t-BuOK | Potassium tert-butoxide |
| t-BuONa | Sodium tert-butoxide |
| T₃P | n-Propylphosphonic cyclic anhydride |
| TMSCN | Trimethylsilyl cyanide |
| TFA | Trifluoroacetic acid |
| TFAA | Trifluoroacetic anhydride |
| THF | Tetrahydrofuran |
| TLC | thin layer chromatography |
| tBuXPhospd-G3 | Methanesulfonato(2-di-t-butylphosphino-2′,4′,6′-tri-i-propyl-1,1′- |
| | biphenyl)(2′-amino-1,1′-biphenyl-2-yl)palladium(II) |
| tBuXPhos | 2-Di-tert-butylphosphino-2′,4′,6′-triisopropylbiphenyl |
| UHP | Urea hydrogen peroxide |
| μL | Microliters |
| XantPhos | 4,5-Bis(diphenylphosphino)-9,9-dimethylxanthene |
| XPhos | 2-Dicyclohexylphosphino-2′,4′,6′-triisopropylbiphenyl |
| 4CzIPN | (4r,6r)-2,4,5,6-tetra(9H-carbazol-9-yl)isophthalonitrile |

### Compound Synthesis

### Example 1a (common intermediate): 7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-ol

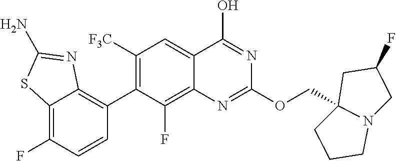 [SMILES: Nc1nc2c(-c3c(C(F)(F)F)cc4c(O)nc(OC[C@@]56CCCN5C[C@H](F)C6)nc4c3F)ccc(F)c2s1]

### Step 1: 7-bromo-2,4-dichloro-8-fluoro-6-(trifluoromethyl)quinazoline

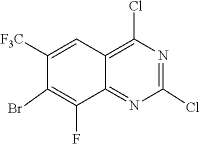 [SMILES: Fc1c(Br)c(C(F)(F)F)cc2c(Cl)nc(Cl)nc12]

To a stirred solution of 7-bromo-8-fluoro-6-(trifluoromethyl)quinazoline-2,4-diol (20.0 g, 61.2 mmol) in 120 mL POCl₃ was added N,N-diisopropylethylamine (40.0 mL), and the resulting mixture was stirred at 100° C. for 2 hrs. The cooled reaction mixture was evaporated under vacuo. The residue was purified by flash chromatography column (PE/DCM=1/1) to give the title compound (16.5 g). MS (ESI, m/e) [M+H]⁺ 364.7.

### Step 2: 4-(benzyloxy)-7-bromo-2-chloro-8-fluoro-6-(trifluoromethyl)quinazoline

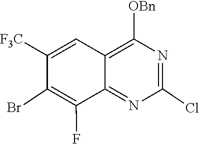 [SMILES: Fc1c(Br)c(C(F)(F)F)cc2c(OCc3ccccc3)nc(Cl)nc12]

To a stirred solution of NaH (2.20 g, 54.4 mmol, 60%) in 50 mL THF was added BnOH (5.15 g, 47.6 mmol) at 0° C. and it was stirred for 30 mins at 0° C. Then, to a solution of 7-bromo-2,4-dichloro-8-fluoro-6-(trifluoromethyl)quinazoline (16.5 g, 45.3 mmol) in 150 mL THF was added the above mixture at −40° C., and the reaction mixture was stirred for 3 hrs at room temperature. The reaction was quenched with water and extracted with EtOAc, and the combined organic layer was dried over sodium sulfate and evaporated. The residue was purified by flash chromatography column to give the title product (16.7 g). MS (ESI, m/e) [M+H]⁺ 436.7.

### Step 3: 4-(benzyloxy)-7-bromo-8-fluoro-2-(((2R,7aS)-2-fluorotetrahydro-1H-pyrrolizin-7a(5H)-yl)methoxy)-6-(trifluoromethyl)quinazoline

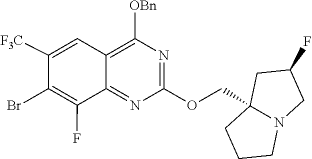 [SMILES: Fc1c(Br)c(C(F)(F)F)cc2c(OCc3ccccc3)nc(OC[C@@]34CCCN3C[C@H](F)C4)nc12]

To a stirred solution of 4-(benzyloxy)-7-bromo-2-chloro-8-fluoro-6-(trifluoromethyl)quinazoline (12.5 g, 28.7 mmol) in 120 mL dioxane was added Cs₂CO₃ (23.4 g, 71.8 mmol) and ((2R,7aS)-2-fluorotetrahydro-1H-pyrrolizin-7a(5H)-yl)methanol (6.85 g, 43.1 mmol), and the resulting mixture was stirred for overnight at 80° C. The cooled reaction mixture was filtered and the filtrate was concentrated under reduced pressure. The residue was purified by flash chromatography column (eluting with DCM/MeOH=20/1) to give the title product (13.1 g). MS (ESI, m/e) [M+H]⁺ 557.8.

### Step 4: tert-butyl (4-(4-(benzyloxy)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl)carbamate

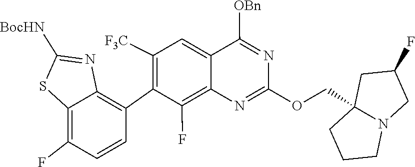 [SMILES: CC(C)(C)OC(=O)Nc1nc2c(-c3c(C(F)(F)F)cc4c(OCc5ccccc5)nc(OC[C@@]56CCCN5C[C@H](F)C6)nc4c3F)ccc(F)c2s1]

To a 250 mL round-bottomed flask was added 4-(benzyloxy)-7-bromo-8-fluoro-2-(((2R,7aS)-2-fluorotetrahydro-1H-pyrrolizin-7a(5H)-yl)methoxy)-6-(trifluoromethyl)quinazoline (10.0 g, 17.9 mmol), (2-((tert-butoxycarbonyl)amino)-7-fluorobenzo[d]thiazol-4-yl)boronic acid (11.2 g, 35.8 mmol), K₃PO₄ (15.2 g, 71.6 mmol), Pd(dtbpf)Cl₂ (1.17 g, 1.79 mmol), dioxane (120 mL) and H₂O (24 mL), and the reaction mixture was stirred at 100° C. for 3 hours. The resulting cooled mixture was concentrated and purified by flash column chromatography (DCM/MeOH=20/1) to give the title product (8.67 g). MS (ESI, m/e) [M+H]⁺ 745.9.

### Step 5: 7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-ol

To a 250 mL round-bottomed flask was added tert-butyl (4-(4-(benzyloxy)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl)carbamate (10.0 g, 13.4 mmol) and TFA (100 mL), and the resulting mixture was stirred at room temperature overnight. The reaction mixture was evaporated under vacuo. The residue was purified by prep-HPLC to give the title compound (6.1 g). MS (ESI, m/e) [M+H]⁺ 556.3.

### Example 1b (Common Intermediate): tert-butyl (7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[d]thiazol-2-yl)carbamate

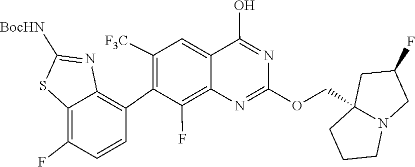 [SMILES: CC(C)(C)OC(=O)Nc1nc2c(-c3c(C(F)(F)F)cc4c(O)nc(OC[C@@]56CCCN5C[C@H](F)C6)nc4c3F)ccc(F)c2s1]

### Step 1: tert-butyl (7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[d]thiazol-2-yl)carbamate

To a stirred solution of tert-butyl (4-(4-(benzyloxy)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl)carbamate (8.67 g, 11.6 mmol) in 120 mL EtOAc was added 10% Pd/C (2.90 g) under N₂, and the resulting mixture was degassed with H₂ for 3 times and stirred for 5 hrs at room temperature under H₂ atmosphere. The reaction mixture was filtered and the filtrate was concentrated under reduced pressure. The residue was purified by flash chromatography column (eluting with DCM/MeOH=10/1) to give the title product (6.54 g). MS (ESI, m/e) [M+1]⁺ 656.3. ¹H NMR (500 MHz, DMSO-d₆) δ 12.20 (s, 1H), 8.18 (s, 1H), 7.46-7.36 (m, 1H), 7.25-7.35 (m, 1H), 5.20-5.45 (m, 1H), 2.80-3.23 (m, 4H), 1.77-2.22 (m, 6H), 1.47 (s, 9H).

### Example 1: 4-(4-(cyclopropylamino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

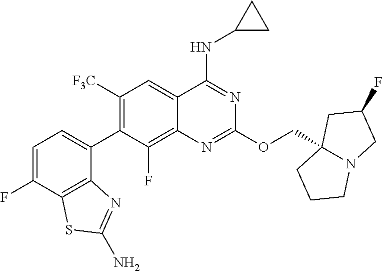 [SMILES: Nc1nc2c(-c3c(C(F)(F)F)cc4c(NC5CC5)nc(OC[C@@]56CCCN5C[C@H](F)C6)nc4c3F)ccc(F)c2s1]

### Step 1: 4-(4-(cyclopropylamino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of 7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-ol (25 mg, 0.04 mmol) in acetonitrile (5 mL) was added cyclopropanamine (5 mg, 0.09 mmol), (benzotriazol-1-yloxy)tris(dimethylamino)phosphonium hexafluorophosphate (36 mg, 0.08 mmol), and 0.2 mL N,N-diisopropylethylamine at room temperature. After the addition, the mixture was stirred at room temperature for 16 hrs. Then, it was diluted with dichloromethane and water. The organic layer was combined, dried over sodium sulphate and evaporated, and the residue was purified by Prep-HPLC to give the title compound (6 mg). ¹H NMR (500 MHz, CD₃OD) δ 8.44 (s, 1H), 7.21-7.14 (m, 1H), 6.99-6.92 (m, 1H), 5.46-5.30 (m, 1H), 4.51-4.35 (m, 2H), 3.55-3.36 (m, 3H), 3.19-3.06 (m, 2H), 2.53-2.18 (m, 3H), 2.16-2.05 (m, 2H), 2.01-1.92 (m, 1H), 1.00-0.89 (m, 2H), 0.82-0.74 (m, 2H). MS (ESI, m/e) [M+H]⁺ 595.4.

### Example 2: 7-fluoro-4-(8-fluoro-4-(((1S,2R)-2-fluorocyclopropyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

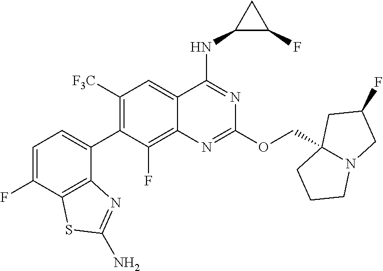 [SMILES: Nc1nc2c(-c3c(C(F)(F)F)cc4c(N[C@H]5C[C@H]5F)nc(OC[C@@]56CCCN5C[C@H](F)C6)nc4c3F)ccc(F)c2s1]

### Step 1: tert-butyl (7-fluoro-4-(8-fluoro-4-(((1S,2R)-2-fluorocyclopropyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-yl)carbamate

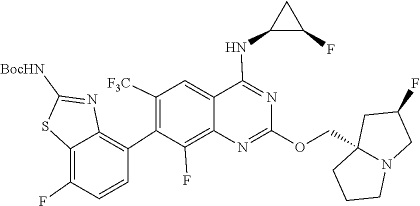 [SMILES: CC(C)(C)OC(=O)Nc1nc2c(-c3c(C(F)(F)F)cc4c(N[C@H]5C[C@H]5F)nc(OC[C@@]56CCCN5C[C@H](F)C6)nc4c3F)ccc(F)c2s1]

To a solution of tert-butyl (7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[d]thiazol-2-yl)carbamate (50 mg, 0.08 mmol) in 10 mL N,N-dimethylformamide was added (1S,2R)-2-fluorocyclopropan-1-amine hydrogen chloride (17 mg, 0.15 mmol), ((1H-benzo[d][1,2,3]triazol-1-yl)oxy)tri(pyrrolidin-1-yl)phosphonium hexafluorophosphate (80 mg, 0.15 mmol) and 0.3 mL N,N-diisopropylethylamine. The reaction was stirred at room temperature for 16 hrs, then it was diluted with ethyl acetate and water. Combined organic layer was dried over sodium sulfate and evaporated. The residue was purified by chromatography on silica to give the title compound (10 mg, 15%). MS (ESI, m/e) [M+H]⁺ 713.4.

### Step 2: 7-fluoro-4-(8-fluoro-4-(((1S,2R)-2-fluorocyclopropyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

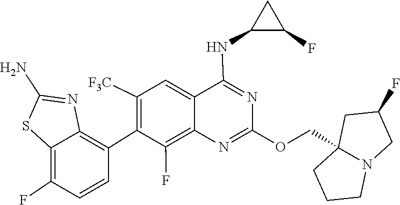 [SMILES: Nc1nc2c(-c3c(C(F)(F)F)cc4c(N[C@H]5C[C@H]5F)nc(OC[C@@]56CCCN5C[C@H](F)C6)nc4c3F)ccc(F)c2s1]

To a solution of tert-butyl (7-fluoro-4-(8-fluoro-4-(((1S,2R)-2-fluorocyclopropyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-yl)carbamate (10 mg, 0.015 mmol) in dichloromethane (2 mL) was added trifluoroacetic acid (1 mL) at room temperature. After the addition, the mixture was stirred at room temperature for 4 hrs. Then the solution was evaporated, and pH of the residue was adjusted to 11 with aq sodium carbonate, and the mixture was diluted with dichloromethane and water. Combined organic layer was dried over sodium sulfate and evaporated. The residue was purified by Prep-HPLC to give the title compound (2 mg). ¹H NMR (500 MHz, CD₃OD) δ 8.55 (s, 1H), 7.22-7.16 (m, 1H), 7.00-6.93 (m, 1H), 5.50-5.33 (m, 1H), 4.95-4.75 (m, 2H), 4.60-4.47 (m, 2H), 3.73-3.45 (m, 3H), 3.17-3.10 (m, 1H), 2.58-2.22 (m, 3H), 2.22-1.94 (m, 3H), 1.42-1.28 (m, 2H). MS (ESI, m/e) [M+H]⁺ 613.4.

### Example 3: 7-fluoro-4-(8-fluoro-2-(((2R,7aS)-2-fluorotetrahydro-1H-pyrrolizin-7a(5H)-yl)methoxy)-4-((2-methylcyclopropyl)amino)-6-(trifluoromethyl)quinazolin-7-yl)benzo[d]thiazol-2-amine

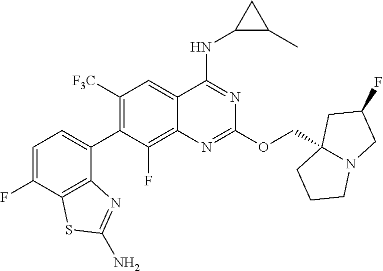 [SMILES: CC1CC1Nc1nc(OC[C@@]23CCCN2C[C@H](F)C3)nc2c(F)c(-c3ccc(F)c4sc(N)nc34)c(C(F)(F)F)cc12]

Example 3 was prepared by similar procedure as described in Example 1 by replacing cyclopropanamine with 2-methylcyclopropan-1-amine to give the title product. ¹H NMR (500 MHz, CD₃OD) δ 8.43 (s, 1H), 7.22-7.15 (m, 1H), 7.00-6.90 (m, 1H), 5.50-5.30 (m, 1H), 4.55-4.35 (m, 2H), 3.65-3.35 (m, 3H), 3.23-3.13 (m, 1H), 2.78-2.70 (m, 1H), 2.53-1.91 (m, 6H), 1.26-0.70 (m, 6H). MS (ESI, m/e) [M+H]⁺ 609.6.

### Example 4: 4-(4-(((trans-2-ethylcyclopropyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

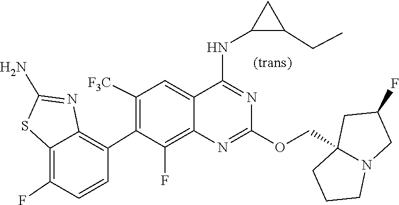 [SMILES: CCC1CC1Nc1nc(OC[C@@]23CCCN2C[C@H](F)C3)nc2c(F)c(-c3ccc(F)c4sc(N)nc34)c(C(F)(F)F)cc12]

Example 4 was prepared by similar procedure as described in Example 1 by replacing cyclopropanamine with trans-2-ethylcyclopropan-1-amine to give the title product. ¹H NMR (500 MHz, CD₃OD) δ 8.44 (s, 1H), 7.19-7.13 (m, 1H), 6.98-6.91 (m, 1H), 5.43-5.27 (m, 1H), 4.47-4.32 (m, 2H), 3.50-3.33 (m, 4H), 3.13-3.05 (m, 1H), 2.88-2.81 (m, 1H), 2.45-1.89 (m, 6H), 158-1.47 (m, 1H), 1.45-1.35 (m, 1H), 1.15-1.05 (m, 3H), 0.97-0.89 (m, 1H), 0.79-0.70 (m, 1H). MS (ESI, m/e) [M+H]⁺ 623.3.

### Example 5: 7-fluoro-4-(8-fluoro-2-(((2R,7aS)-2-fluorotetrahydro-1H-pyrrolizin-7a(5H)-yl)methoxy)-4-(((trans)-2-phenylcyclopropyl)amino)-6-(trifluoromethyl)quinazolin-7-yl)benzo[d]thiazol-2-amine

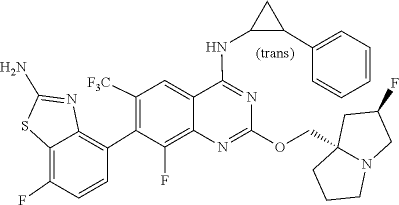 [SMILES: Nc1nc2c(-c3c(C(F)(F)F)cc4c(NC5CC5c5ccccc5)nc(OC[C@@]56CCCN5C[C@H](F)C6)nc4c3F)ccc(F)c2s1]

Example 5 was prepared by similar procedure as described in Example 1 by replacing cyclopropanamine with trans-2-phenylcyclopropan-1-amine to give the title product. ¹H NMR (500 MHz, CD₃OD) δ 8.53-8.45 (m, 1H), 7.41-7.06 (m, 6H), 7.01-6.90 (m, 1H), 5.45-5.25 (m, 1H), 4.28-4.00 (m, 2H), 3.62-3.37 (m, 3H), 3.18-2.99 (m, 1H), 2.39-1.68 (m, 7H), 1.66-1.50 (m, 1H), 1.48-1.34 (m, 1H). MS (ESI, m/e) [M+H]⁺ 671.5.

### Example 6: 7-fluoro-4-(8-fluoro-2-(((2R,7aS)-2-fluorotetrahydro-1H-pyrrolizin-7a(5H)-yl)methoxy)-4-((2-methoxycyclopropyl)amino)-6-(trifluoromethyl)quinazolin-7-yl)benzo[d]thiazol-2-amine

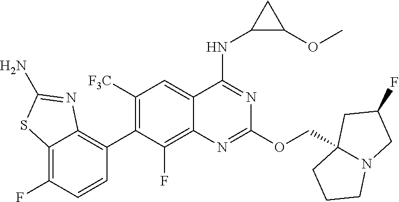 [SMILES: COC1CC1Nc1nc(OC[C@@]23CCCN2C[C@H](F)C3)nc2c(F)c(-c3ccc(F)c4sc(N)nc34)c(C(F)(F)F)cc12]

Example 6 was prepared by similar procedure as described in Example 1 by replacing cyclopropanamine with 2-methoxycyclopropan-1-amine to give the title product. ¹H NMR (500 MHz, CD₃OD) δ 8.54 (s, 1H), 7.23-7.14 (m, 1H), 7.00-6.92 (m, 1H), 5.48-5.29 (m, 1H), 4.55-4.38 (m, 2H), 3.64-3.40 (m, 4H), 3.37-3.32 (m, 3H), 3.24-3.15 (m, 2H), 2.52-1.91 (m, 6H), 1.19-1.08 (m, 2H). MS (ESI, m/e) [M+H]⁺ 625.4.

### Example 7: 4-(4-(((1R,5S,6r)-3-oxabicyclo [3.1.0]hexan-6-yl) amino)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d]thiazol-2-amine

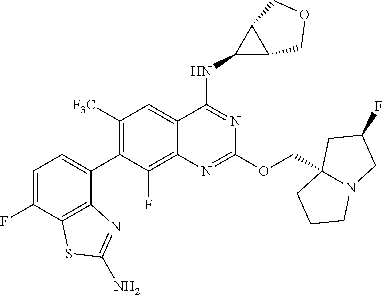 [SMILES: Nc1nc2c(-c3c(C(F)(F)F)cc4c(N[C@H]5[C@@H]6COC[C@@H]65)nc(OC[C@@]56CCCN5C[C@H](F)C6)nc4c3F)ccc(F)c2s1]

Example 7 was prepared by similar procedure as described in Example 1 by replacing cyclopropanamine with (1R,5S,6r)-3-oxabicyclo [3.1.0]hexan-6-amine to give the title product. ¹H NMR (500 MHz, CD₃OD) δ 8.45 (s, 1H), 7.20-7.15 (m, 1H), 6.98-6.94 (m, 1H), 5.50-5.39 (m, 1H), 4.59-4.50 (m, 2H), 4.10-4.08 (m, 2H), 3.83-3.71 (m, 2H), 3.74-3.61 (m, 3H), 3.27-3.23 (m, 1H), 2.87-2.85 (m, 1H), 2.57-2.38 (m, 2H), 2.33-2.25 (m, 1H), 2.20-2.18 (m, 2H), 2.11-1.98 (m, 3H). MS (ESI, m/e) [M+H]⁺ 637.4.

### Example 8: 4-(4-(bicyclo[3.1.0]hexan-6-ylamino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

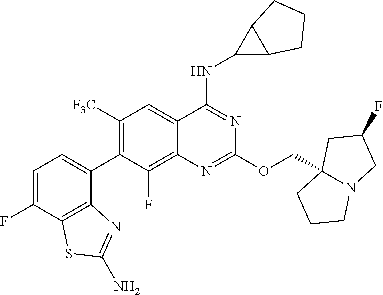 [SMILES: Nc1nc2c(-c3c(C(F)(F)F)cc4c(NC5C6CCCC65)nc(OC[C@@]56CCCN5C[C@H](F)C6)nc4c3F)ccc(F)c2s1]

Example 8 was prepared by similar procedure as described in Example 1 by replacing cyclopropanamine with bicyclo[3.1.0]hexan-6-amine to give the title product. ¹H NMR (500 MHz, CD₃OD) δ 8.48 (s, 1H), 7.20-7.15 (m, 1H), 6.99-6.93 (m, 1H), 5.60-5.41 (m, 1H), 4.72-4.55 (m, 2H), 3.94-3.66 (m, 3H), 3.42-3.34 (m, 1H), 2.85-2.78 (m, 1H), 2.66-2.44 (m, 2H), 2.40-2.23 (m, 3H), 2.22-1.95 (m, 3H), 1.89-1.80 (m, 2H), 1.74-1.58 (m, 3H), 1.37-1.20 (m, 1H). MS (ESI, m/e) [M+H]⁺ 635.4.

### Example 9: 1-((1R,5S,6s)-6-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)-3-azabicyclo[3.1.0]hexan-3-yl)ethan-1-one

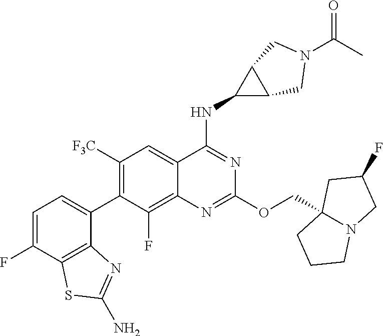 [SMILES: CC(=O)N1C[C@@H]2[C@H](C1)[C@H]2Nc1nc(OC[C@@]23CCCN2C[C@H](F)C3)nc2c(F)c(-c3ccc(F)c4sc(N)nc34)c(C(F)(F)F)cc12]

Example 9 was prepared by similar procedure as described in Example 1 by replacing cyclopropanamine with 1-((1R,5S,6s)-6-amino-3-azabicyclo[3.1.0]hexan-3-yl)ethan-1-one to give the title product. ¹H NMR (500 MHz, CD₃OD) δ 8.44 (s, 1H), 7.25-7.06 (m, 1H), 7.00-6.88 (m, 1H), 5.51-5.33 (m, 1H), 4.60-4.45 (m, 2H), 3.98-3.86 (m, 2H), 3.85-3.74 (m, 1H), 3.71-3.41 (m, 4H), 3.28-3.22 (m, 2H), 2.80-2.75 (m, 1H), 2.59-2.23 (m, 3H), 2.21-1.88 (m, 7H). MS (ESI, m/e) [M+H]⁺ 678.4.

### Example 10: 4-(4-(cyclobutylamino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

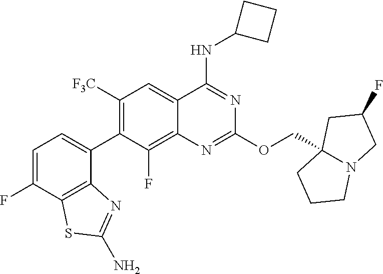 [SMILES: Nc1nc2c(-c3c(C(F)(F)F)cc4c(NC5CCC5)nc(OC[C@@]56CCCN5C[C@H](F)C6)nc4c3F)ccc(F)c2s1]

Example 10 was prepared by similar procedure as described in Example 1 by replacing cyclopropanamine with cyclobutanamine to give the title product. ¹H NMR (500 MHz, CD₃OD) δ 8.54 (s, 1H), 7.20-7.15 (m, 1H), 6.99-6.92 (m, 1H), 5.46-5.30 (m, 1H), 4.41-4.36 (m, 2H), 3.55-3.37 (m, 3H), 3.18-3.08 (m, 2H), 2.53-2.18 (m, 7H), 2.16-1.81 (m, 5H). MS (ESI, m/e) [M+H]⁺ 609.3.

### Example 11: cis-3-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclobutan-1-ol

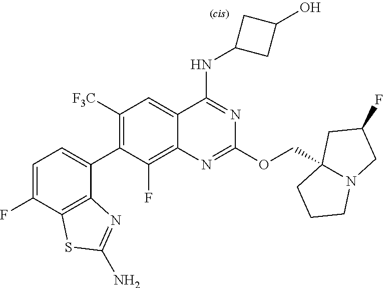 [SMILES: Nc1nc2c(-c3c(C(F)(F)F)cc4c(NC5CC(O)C5)nc(OC[C@@]56CCCN5C[C@H](F)C6)nc4c3F)ccc(F)c2s1]

Example 11 was prepared by similar procedure as described in Example 1 by replacing cyclopropanamine with cis-3-aminocyclobutanol to give the title product. ¹H NMR (500 MHz, CD₃OD) δ 8.61-8.54 (m, 1H), 7.22-7.13 (m, 1H), 7.02-6.89 (m, 1H), 5.49-5.34 (m, 1H), 4.55-4.38 (m, 2H), 4.33-4.09 (m, 2H), 3.66-3.49 (m, 3H), 3.27-3.20 (m, 1H), 2.94-2.83 (m, 2H), 2.53-2.35 (m, 2H), 2.31-2.23 (m, 1H), 2.21-2.08 (m, 4H), 2.05-1.94 (m, 1H). MS (ESI, m/e) [M+H]⁺ 625.6.

### Example 12: (trans)-3-((7-(2-amino-7-fluorobenzo[d]thiazol-4-yl)-8-fluoro-2(((2R,7aS)-2-fluorotetrahydro-1H-pyrrolizin-7a(5H)-yl)methoxy)-6-(trifluoromethyl)quinazolin-4-yl)amino)cyclobutan-1-ol

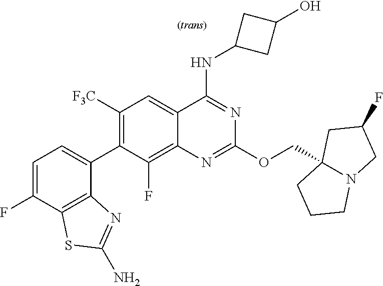 [SMILES: Nc1nc2c(-c3c(C(F)(F)F)cc4c(NC5CC(O)C5)nc(OC[C@@]56CCCN5C[C@H](F)C6)nc4c3F)ccc(F)c2s1]

Example 12 was prepared by similar procedure as described in Example 1 by replacing cyclopropanamine with trans-3-aminocyclobutanol to give the title product. ¹H NMR (500 MHz, CD₃OD) δ 8.61 (s, 1H), 7.21-7.15 (m, 1H), 7.00-6.93 (m, 1H), 5.55-5.40 (m, 1H), 4.63-4.47 (m, 3H), 3.85-3.63 (m, 3H), 2.58-2.00 (m, 10H). MS (ESI, m/e) [M+H]⁺ 625.5.

### Example 13: 7-fluoro-4-(8-fluoro-2-(((2R,7aS)-2-fluorotetrahydro-1H-pyrrolizin-7a(5H)-yl)methoxy)-4-(((cis)-3-methoxycyclobutyl)amino)-6-(trifluoromethyl)quinazolin-7-yl)benzo[d]thiazol-2-amine

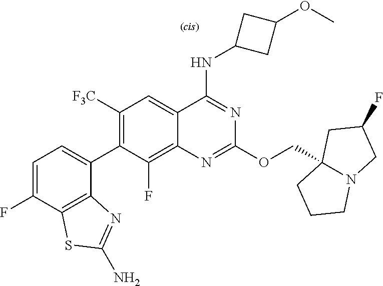 [SMILES: COC1CC(Nc2nc(OC[C@@]34CCCN3C[C@H](F)C4)nc3c(F)c(-c4ccc(F)c5sc(N)nc45)c(C(F)(F)F)cc23)C1]

Example 13 was prepared by similar procedure as described in Example 1 by replacing cyclopropanamine with cis-3-methoxycyclobutanamine to give the title product. 1H NMR (500 MHz, CD₃OD) δ 8.60 (s, 1H), 7.28-7.10 (m, 1H), 7.11-6.84 (m, 1H), 5.59-5.42 (m, 1H), 4.70-4.55 (m, 2H), 4.45-4.35 (m, 1H), 3.96-3.70 (m, 5H), 3.43-3.37 (m, 2H), 2.94-2.84 (m, 2H), 2.68-2.64 (m, 1H), 2.63-2.49 (m, 2H), 2.42-2.36 (m, 1H), 2.33-2.24 (m, 2H), 2.20-2.06 (m, 3H). MS (ESI, m/e) [M+H]⁺ 638.2.

### Example 14: 7-fluoro-4-(8-fluoro-2-(((2R,7aS)-2-fluorotetrahydro-1H-pyrrolizin-7a(5H)-yl)methoxy)-4-(((trans)-3-methoxycyclobutyl)amino)-6-(trifluoromethyl)quinazolin-7-yl)benzo[d]thiazol-2-amine

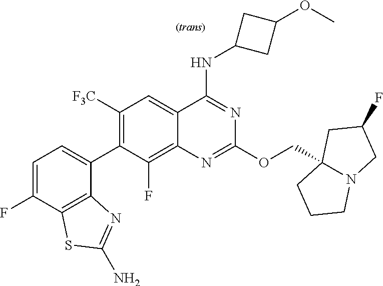 [SMILES: COC1CC(Nc2nc(OC[C@@]34CCCN3C[C@H](F)C4)nc3c(F)c(-c4ccc(F)c5sc(N)nc45)c(C(F)(F)F)cc23)C1]

Example 14 was prepared by similar procedure as described in Example 1 by replacing cyclopropanamine with trans-3-methoxycyclobutanamine to give the title product. 1H NMR (500 MHz, CD₃OD) δ 8.57 (s, 1H), 7.21-7.14 (m, 1H), 6.99-6.92 (m, 1H), 5.58-5.31 (m, 1H), 4.50-4.35 (m, 2H), 4.20-4.10 (m, 1H), 3.62-3.46 (m, 3H), 2.20-3.12 (m, 1H), 2.55-1.96 (m, 9H). MS (ESI, m/e) [M+H]⁺ 639.5.

### Example 15: 7-fluoro-4-(8-fluoro-4-(((cis)-3-fluorocyclobutyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

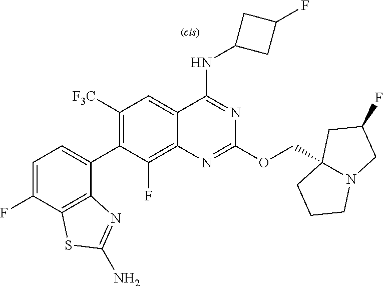 [SMILES: Nc1nc2c(-c3c(C(F)(F)F)cc4c(NC5CC(F)C5)nc(OC[C@@]56CCCN5C[C@H](F)C6)nc4c3F)ccc(F)c2s1]

Example 15 was prepared by similar procedure as described in Example 1 by replacing cyclopropanamine with cis-3-fluorocyclobutanamine to give the title product. ¹H NMR (500 MHz, CD₃OD) δ 8.51 (s, 1H), 7.17-7.05 (m, 1H), 6.95-6.78 (m, 1H), 5.47-5.35 (m, 1H), 4.61-4.19 (m, 3H), 3.90-3.69 (m, 3H), 3.36-3.29 (m, 2H), 2.96-2.84 (m, 2H), 2.53-2.28 (m, 5H), 2.26-2.16 (m, 2H), 2.06-1.96 (m, 1H). MS (ESI, m/e) [M+H]⁺ 627.6.

### Example 16: 7-fluoro-4-(8-fluoro-4-(((trans)-3-fluorocyclobutyl)amino)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

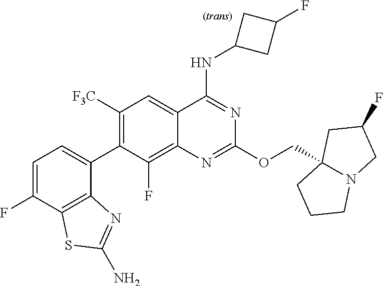 [SMILES: Nc1nc2c(-c3c(C(F)(F)F)cc4c(NC5CC(F)C5)nc(OC[C@@]56CCCN5C[C@H](F)C6)nc4c3F)ccc(F)c2s1]

Example 16 was prepared by similar procedure as described in Example 1 by replacing cyclopropanamine with trans-3-fluorocyclobutanamine to give the title product. ¹H NMR (500 MHz, CD₃OD) δ 8.55 (s, 1H), 7.21-7.14 (m, 1H), 7.00-6.92 (m, 1H), 5.48-5.24 (m, 2H), 5.01-4.92 (m, 1H), 4.51-4.36 (m, 2H), 3.63-3.46 (m, 3H), 3.28-3.16 (m, 1H), 2.80-1.96 (m, 10H). MS (ESI, m/e) [M+H]⁺ 627.4.

### Example 17: 4-(4-((3,3-difluorocyclobutyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

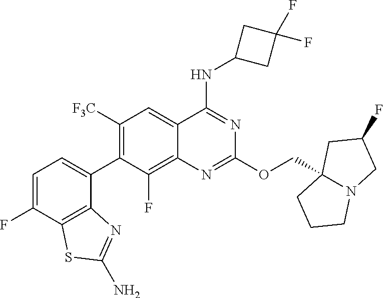 [SMILES: Nc1nc2c(-c3c(C(F)(F)F)cc4c(NC5CC(F)(F)C5)nc(OC[C@@]56CCCN5C[C@H](F)C6)nc4c3F)ccc(F)c2s1]

Example 17 was prepared by similar procedure as described in Example 1 by replacing cyclopropanamine with 3,3-difluorocyclobutan-1-amine to give the title product. ¹H NMR (500 MHz, CD₃OD) δ 8.57 (s, 1H), 7.24-7.07 (m, 1H), 7.02-6.91 (m, 1H), 5.52-5.37 (m, 1H), 4.65-4.30 (m, 3H), 3.82-3.50 (m, 3H), 3.20-3.04 (m, 2H), 2.96-2.78 (m, 2H), 2.60-2.37 (m, 2H), 2.37-2.26 (m, 1H), 2.20 (m, 2H), 2.03 (m, 1H). MS (ESI, m/e) [M+H]⁺ 645.4.

### Example 18: (cis)-3-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclobutane-1-carbonitrile

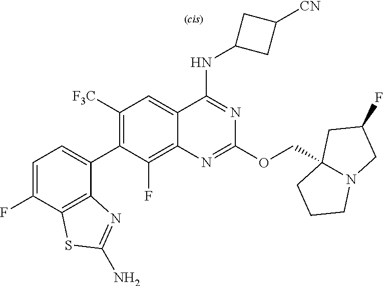 [SMILES: N#CC1CC(Nc2nc(OC[C@@]34CCCN3C[C@H](F)C4)nc3c(F)c(-c4ccc(F)c5sc(N)nc45)c(C(F)(F)F)cc23)C1]

Example 18 was prepared by similar procedure as described in Example 1 by replacing cyclopropanamine with cis-3-aminocyclobutanecarbonitrile to give the title product. ¹H NMR (500 MHz, CD₃OD) δ 8.55 (s, 1H), 7.26-7.10 (m, 1H), 7.00-6.90 (m, 1H), 5.54-5.37 (m, 1H), 4.60-4.46 (m, 2H), 3.77-3.61 (m, 3H), 3.48-3.40 (m, 1H), 3.19-3.09 (m, 1H), 3.00-2.91 (m, 2H), 2.67-2.58 (m, 2H), 2.60-2.42 (m, 2H), 2.36-2.29 (m, 1H), 2.27-2.15 (m, 2H), 2.08-2.00 (m, 1H). MS (ESI, m/e) [M+H]⁺ 634.2.

### Example 19: (trans)-3-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclobutane-1-carbonitrile

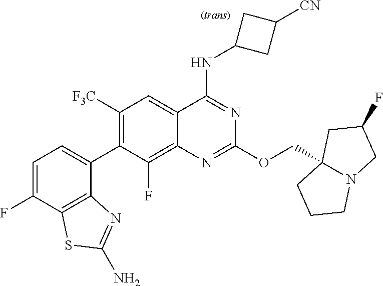 [SMILES: N#CC1CC(Nc2nc(OC[C@@]34CCCN3C[C@H](F)C4)nc3c(F)c(-c4ccc(F)c5sc(N)nc45)c(C(F)(F)F)cc23)C1]

Example 19 was prepared by similar procedure as described in Example 1 by replacing cyclopropanamine with trans-3-aminocyclobutanecarbonitrile to give the title product. ¹H NMR (500 MHz, CD₃OD) δ 8.59 (s, 1H), 7.19-7.13 (m, 1H), 7.00-6.91 (m, 1H), 5.48-5.33 (m, 1H), 4.54-4.40 (m, 2H), 3.75-3.46 (m, 3H), 3.28-3.23 (m, 2H), 2.56-1.96 (m, 10H). MS (ESI, m/e) [M+H]⁺ 639.5.

### Example 20: ((cis)-3-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclobutyl)methanol

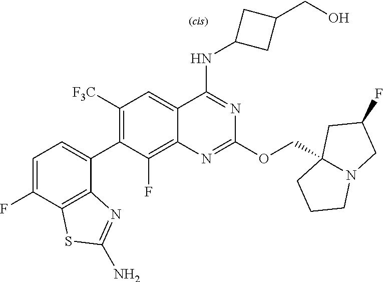 [SMILES: Nc1nc2c(-c3c(C(F)(F)F)cc4c(NC5CC(CO)C5)nc(OC[C@@]56CCCN5C[C@H](F)C6)nc4c3F)ccc(F)c2s1]

Example 20 was prepared by similar procedure as described in Example 1 by replacing cyclopropanamine with cis-3-amino-cyclobutanemethanol to give the title product. ¹H NMR (500 MHz, CD₃OD) δ 8.58 (s, 1H), 7.26-7.12 (m, 1H), 7.04-6.88 (m, 1H), 5.58-5.40 (m, 1H), 4.76-4.50 (m, 3H), 3.81-3.51 (m, 4H), 2.64-1.95 (m, 11H). MS (ESI, m/e) [M+H]⁺ 639.2.

### Example 21: ((trans)-3-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclobutyl)methanol

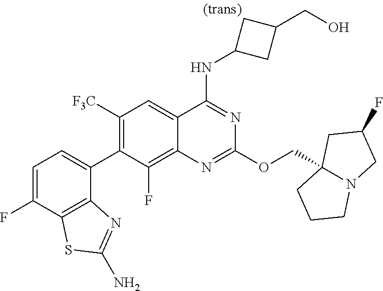 [SMILES: Nc1nc2c(-c3c(C(F)(F)F)cc4c(NC5CC(CO)C5)nc(OC[C@@]56CCCN5C[C@H](F)C6)nc4c3F)ccc(F)c2s1]

Example 21 was prepared by similar procedure as described in Example 1 by replacing cyclopropanamine with trans-3-amino-cyclobutanemethanol to give the title product. ¹H NMR (500 MHz, CD₃OD) δ 8.59 (s, 1H), 7.22-7.14 (m, 1H), 7.00-6.92 (m, 1H), 5.50-5.36 (m, 1H), 4.54-4.40 (m, 2H), 3.75-3.56 (m, 5H), 2.56-1.96 (m, 11H). MS (ESI, m/e) [M+H]⁺ 639.5.

### Example 22: 7-fluoro-4-(8-fluoro-2-(((2R,7aS)-2-fluorotetrahydro-1H-pyrrolizin-7a(5H)-yl)methoxy)-4-(spiro[3.3]heptan-2-ylamino)-6-(trifluoromethyl)quinazolin-7-yl)benzo[d]thiazol-2-amine

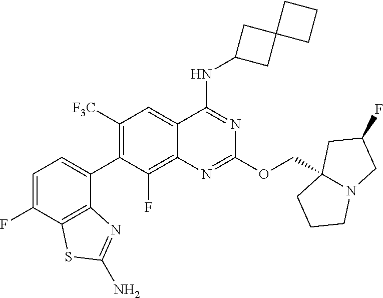 [SMILES: Nc1nc2c(-c3c(C(F)(F)F)cc4c(NC5CC6(CCC6)C5)nc(OC[C@@]56CCCN5C[C@H](F)C6)nc4c3F)ccc(F)c2s1]

Example 22 was prepared by similar procedure as described in Example 1 by replacing cyclopropanamine with spiro[3.3]heptan-2-amine to give the title product. ¹H NMR (500 MHz, CD₃OD) δ 8.54 (s, 1H), 7.25-7.08 (m, 1H), 7.00-6.90 (m, 1H), 5.48-5.30 (m, 1H), 4.70-4.55 (m, 1H), 4.50-4.30 (m, 2H), 3.69-3.39 (m, 3H), 3.25-3.12 (m, 1H), 2.61-2.51 (m, 2H), 2.51-2.31 (m, 2H), 2.30-2.08 (m, 7H), 2.07-1.95 (m, 3H), 1.95-1.80 (m, 2H). MS (ESI, m/e) [M+H]⁺ 649.4.

### Example 23: 4-(4-((2-oxaspiro[3.3]heptan-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

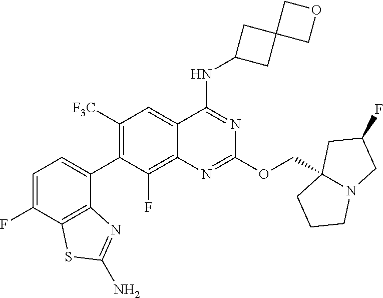 [SMILES: Nc1nc2c(-c3c(C(F)(F)F)cc4c(NC5CC6(COC6)C5)nc(OC[C@@]56CCCN5C[C@H](F)C6)nc4c3F)ccc(F)c2s1]

Example 23 was prepared by similar procedure as described in Example 1 by replacing cyclopropanamine with 2-oxaspiro[3.3]heptan-6-amine to give the title product. ¹H NMR (500 MHz, CD₃OD) δ 8.51 (s, 1H), 7.25-7.07 (m, 1H), 7.00-6.90 (m, 1H), 5.49-5.33 (m, 1H), 4.72-4.36 (m, 5H), 3.72-3.42 (m, 3H), 3.25-3.12 (m, 1H), 2.93-2.76 (m, 2H), 2.55-2.33 (m, 4H), 2.32-2.21 (m, 1H), 2.20-2.08 (m, 2H), 2.06-1.90 (m, 1H). MS (ESI, m/e) [M+H]⁺ 651.4.

### Example 24: 4-(4-(bicyclo [1.1.1]pentan-1-ylamino)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d]thiazol-2-amine

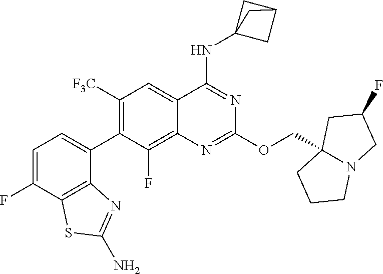 [SMILES: Nc1nc2c(-c3c(C(F)(F)F)cc4c(NC56CC(C5)C6)nc(OC[C@@]56CCCN5C[C@H](F)C6)nc4c3F)ccc(F)c2s1]

Example 24 was prepared by similar procedure as described in Example 1 by replacing cyclopropanamine with bicyclo[1.1.1]pentan-1-amine to give the title product. ¹H NMR (500 MHz, CD₃OD) δ 8.48 (s, 1H), 7.21-7.13 (m, 1H), 6.97-6.94 (m, 1H), 5.49-5.38 (m, 1H), 4.52-4.44 (m, 2H), 3.73-3.48 (m, 3H), 3.29-3.21 (m, 1H), 2.65-1.98 (m, 13H). MS (ESI, m/e) [M+H]⁺ 621.5.

### Example 25: 7-fluoro-4-(8-fluoro-4-(((1S,3R)-3-fluorocyclopentyl) amino)-2-(((2R,7aS)-2-fluorotetrahydro-1H-pyrrolizin-7a(5H)-yl) methoxy)-6-(trifluoromethyl) quinazolin-7-yl) benzo[d]thiazol-2-amine

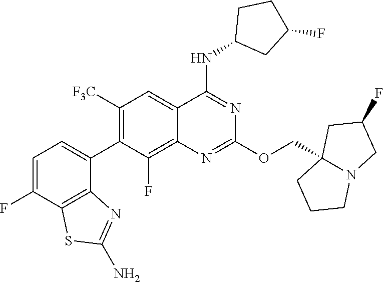 [SMILES: Nc1nc2c(-c3c(C(F)(F)F)cc4c(N[C@@H]5CC[C@H](F)C5)nc(OC[C@@]56CCCN5C[C@H](F)C6)nc4c3F)ccc(F)c2s1]

Example 25 was prepared by similar procedure as described in Example 1 by replacing cyclopropanamine with (1S,3R)-3-fluorocyclopentan-1-amine to give the title product. ¹H NMR (500 MHz, CD₃OD) δ 8.62 (s, 1H), 7.23-7.13 (m, 1H), 6.98-6.94 (m, 1H), 5.47-5.15 (m, 2H), 4.77-4.69 (m, 1H), 4.56-4.40 (m, 2H), 3.72-3.48 (m, 3H), 3.28-3.18 (m, 1H), 2.66-2.35 (m, 3H), 2.31-2.23 (m, 2H), 2.22-1.82 (m, 7H). MS (ESI, m/e) [M+H]⁺ 641.5.

### Example 26: (1S,2R)-2-((7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)amino)cyclopentane-1-carbonitrile

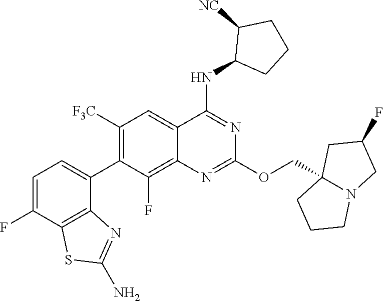 [SMILES: N#C[C@H]1CCC[C@H]1Nc1nc(OC[C@@]23CCCN2C[C@H](F)C3)nc2c(F)c(-c3ccc(F)c4sc(N)nc34)c(C(F)(F)F)cc12]

Example 26 was prepared by similar procedure as described in Example 1 by replacing cyclopropanamine with (1S,2R)-2-aminocyclopentane-1-carbonitrile to give the title product. ¹H NMR (500 MHz, CD₃OD) δ 8.71 (s, 1H), 7.25-7.13 (m, 1H), 7.01-6.92 (m, 1H), 5.50-5.33 (m, 1H), 4.58-4.35 (m, 2H), 3.71-3.43 (m, 4H), 3.27-3.15 (m, 1H), 2.56-2.34 (m, 2H), 2.33-2.21 (m, 3H), 2.20-1.96 (m, 6H), 1.86-1.71 (m, 1H). MS (ESI, m/e) [M+H]⁺ 648.4.

### Example 27: 4-(4-(cyclohexylamino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

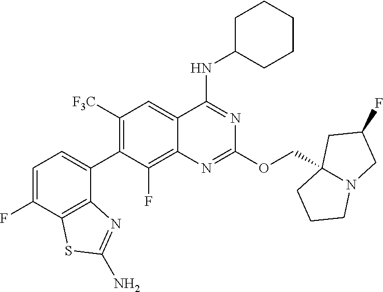 [SMILES: Nc1nc2c(-c3c(C(F)(F)F)cc4c(NC5CCCCC5)nc(OC[C@@]56CCCN5C[C@H](F)C6)nc4c3F)ccc(F)c2s1]

Example 27 was prepared by similar procedure as described in Example 1 by replacing cyclopropanamine with cyclohexamine to give the title product. ¹H NMR (500 MHz, CD₃OD) δ 8.57 (s, 1H), 7.23-7.13 (m, 1H), 7.01-6.92 (m, 1H), 5.52-5.36 (m, 1H), 4.60-4.44 (m, 2H), 4.33-4.24 (m, 1H), 3.80-3.54 (m, 3H), 2.56-1.70 (m, 11H), 1.59-1.50 (m, 4H), 1.34-1.22 (m, 1H). MS (ESI, m/e) [M+H]⁺ 637.4.

### Example 28a (Common Intermediate): tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[d]thiazol-2-yl)carbamate

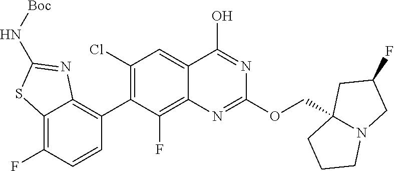 [SMILES: CC(C)(C)OC(=O)Nc1nc2c(-c3c(Cl)cc4c(O)nc(OC[C@@]56CCCN5C[C@H](F)C6)nc4c3F)ccc(F)c2s1]

### Step 1: 4-(benzyloxy)-7-bromo-2,6-dichloro-8-fluoroquinazoline

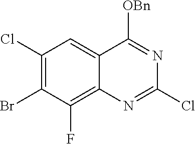 [SMILES: Fc1c(Br)c(Cl)cc2c(OCc3ccccc3)nc(Cl)nc12]

To the solution of benzyl alcohol (3.27 g, 30.27 mmol) in THF (200 mL) was added NaH (1.33 g, 33.3 mmol) in dropwise at 0° C., and the mixture was stirred at 0° C. for another 30 min. The mixture was cooled to −40° C., and 7-bromo-2,4,6-trichloro-8-fluoroquinazoline (10 g, 30.27 mmol) was added. The mixture was stirred at room temperature overnight. The mixture was diluted with water, filtered, and the solid was collected to give the title product (11 g, 91%). MS (ESI, m/e) [M+H]⁺=400.

### Step 2: 4-(benzyloxy)-7-bromo-6-chloro-8-fluoro-2-(((2R,7aS)-2-fluorotetrahydro-1H-pyrrolizin-7a(5H)-yl)methoxy)quinazoline

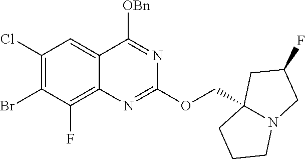 [SMILES: Fc1c(Br)c(Cl)cc2c(OCc3ccccc3)nc(OC[C@@]34CCCN3C[C@H](F)C4)nc12]

4-(benzyloxy)-7-bromo-2,6-dichloro-8-fluoroquinazoline (11 g, 27.5 mmol), ((2R,7aS)-2-fluorotetrahydro-1H-pyrrolizin-7a(5H)-yl)methanol (4.4 g, 27.5 mmol), RuPhos Pd G3 (2.3 g, 2.8 mmol) and Cs₂CO₃ (17.9 g, 55 mmol) were placed in dioxane (110 mL). The mixture was stirred for 16 hrs and then cooled to room temperature. Solvents were removed and the residue was purified by column chromatography (hexane/EtOAc=2/1) to give the title product (9 g). MS (ESI, m/e) [M+H]⁺=524.3.

### Step 3: tert-butyl (4-(4-(benzyloxy)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-yl)carbamate

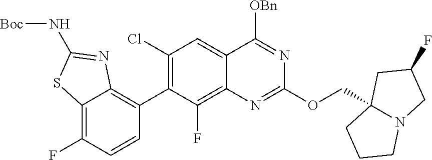 [SMILES: CC(C)(C)OC(=O)Nc1nc2c(-c3c(Cl)cc4c(OCc5ccccc5)nc(OC[C@@]56CCCN5C[C@H](F)C6)nc4c3F)ccc(F)c2s1]

4-(benzyloxy)-7-bromo-6-chloro-8-fluoro-2-(((2R,7aS)-2-fluorotetrahydro-1H-pyrrolizin-7a(5H)-yl)methoxy)quinazoline (9 g, 17.2 mmol), (2-((tert-butoxycarbonyl)amino)-7-fluorobenzo[d]thiazol-4-yl)boronic acid (8.1 g, 25.8 mmol), Pd(dppf)Cl₂ (1.2 g, 1.7 mmol) and NaHCO₃ (2.9 g, 34.4 mmol) were placed in dioxane (90 mL). The mixture was stirred at 80° C. for 4 hrs and then cooled to room temperature. Solvents were removed and the residue was purified by column chromatography (hexane/EtOAc=1/2) to give the title product (8.8 g). MS (ESI, m/e) [M+H]=712.4.

### Step 4: tert-butyl (4-(6-chloro-8-fluoro-2-(((2R,7aS)-2-fluorotetrahydro-1H-pyrrolizin-7a(5H)-yl)methoxy)-4-oxo-3,4-dihydroquinazolin-7-yl)-7-fluorobenzo[d]thiazol-2-yl)carbamate

tert-butyl (4-(4-(benzyloxy)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-yl)carbamate (8.8 g, 12.4 mmol) were placed in EtOAc (100 mL), and the mixture was stirred at 50° C. for 16 hrs. Solvents were removed and the residue was purified by column chromatography (DCM/MeOH=10/1) to give the title product (4.6 g, 60%). MS (ESI, m/e) [M+H]⁺=622.3.

### Example 28: 4-(6-chloro-4-(cyclopropylamino)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

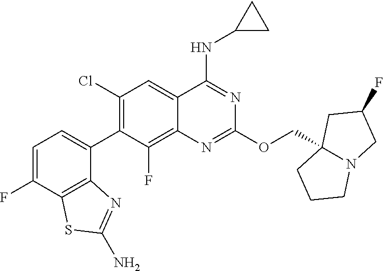 [SMILES: Nc1nc2c(-c3c(Cl)cc4c(NC5CC5)nc(OC[C@@]56CCCN5C[C@H](F)C6)nc4c3F)ccc(F)c2s1]

Example 28 was prepared by similar procedure as described in Example 2 by replacing tert-butyl (7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[d]thiazol-2-yl)carbamate and (1S,2R)-2-fluorocyclopropan-1-amine with tert-butyl (4-(6-chloro-8-fluoro-2-(((2R,7aS)-2-fluorotetrahydro-1H-pyrrolizin-7a(5H)-yl)methoxy)-4-oxo-3,4-dihydroquinazolin-7-yl)-7-fluorobenzo[d]thiazol-2-yl)carbamate and cyclopropanamine to give the title product. ¹H NMR (500 MHz, DMSO-d₆) δ 10.87 (s, 1H), 8.64 (s, 1H), 8.34 (s, 1H), 7.91 (s, 2H), 7.27-7.16 (m, 1H), 7.10-7.01 (m, 1H), 5.62-5.48 (m, 1H), 4.62-4.56 (m, 2H), 3.90-3.72 (m, 3H), 3.36-3.28 (m, 1H), 3.18-3.10 (m, 1H), 2.35-2.20 (m, 2H), 2.23-2.08 (m, 2H), 2.08-1.98 (m, 1H), 0.89-0.70 (m, 4H). MS (ESI, m/e) [M+H]⁺ 561.5.

### Example 29: 4-(4-(((1R,5S,6r)-3-oxabicyclo[3.1.0]hexan-6-yl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

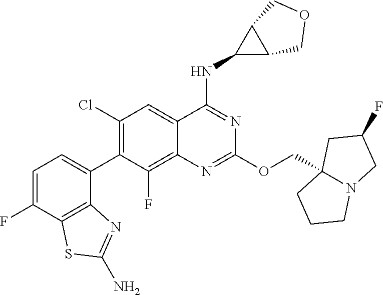 [SMILES: Nc1nc2c(-c3c(Cl)cc4c(N[C@H]5[C@@H]6COC[C@@H]65)nc(OC[C@@]56CCCN5C[C@H](F)C6)nc4c3F)ccc(F)c2s1]

Example 29 was prepared by similar procedure as described in Example 28 by replacing cyclopropanamine with (1R,5S,6r)-3-oxabicyclo [3.1.0]hexan-6-amine to give the title product. ¹H NMR (500 MHz, DMSO-d₆) δ 8.56 (s, 1H), 8.27 (s, 1H), 7.91 (s, 2H), 7.28-7.16 (m, 1H), 7.10-7.00 (m, 1H), 5.49-5.28 (m, 1H), 4.20-4.15 (m, 2H), 4.00-3.88 (m, 2H), 3.79-3.63 (m, 2H), 3.25-3.05 (m, 2H), 2.98-2.84 (m, 2H), 2.30-2.10 (m, 2H), 2.11-1.98 (m, 3H), 1.95-1.75 (m, 3H). MS (ESI, m/e) [M+H]⁺ 603.5.

### Example 30: 1-((1R,5S,6s)-6-((7-(2-amino-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)amino)-3-azabicyclo[3.1.0]hexan-3-yl)ethan-1-one

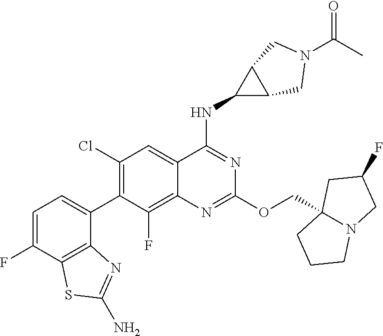 [SMILES: CC(=O)N1C[C@@H]2[C@H](C1)[C@H]2Nc1nc(OC[C@@]23CCCN2C[C@H](F)C3)nc2c(F)c(-c3ccc(F)c4sc(N)nc34)c(Cl)cc12]

Example 30 was prepared by similar procedure as described in Example 28 by replacing cyclopropanamine with 1-((1R,5S,6s)-6-amino-3-azabicyclo[3.1.0]hexan-3-yl)ethan-1-one to give the title product. ¹H NMR (500 MHz, DMSO-d₆) δ 8.60 (s, 1H), 8.26 (s, 1H), 7.91 (s, 2H), 7.31-7.17 (m, 1H), 7.10-7.00 (m, 1H), 5.47-5.23 (m, 1H), 4.20-4.08 (m, 2H), 3.80-3.64 (m, 3H), 3.47-3.36 (m, 1H), 3.30-3.28 (m, 1H), 3.23-3.00 (m, 2H), 2.87 (s, 1H), 2.76 (s, 1H), 2.25-2.00 (m, 4H), 2.00-1.66 (m, 7H). MS (ESI, m/e) [M+H]⁺ 644.5.

### Example 31: 4-(6-chloro-4-(cyclobutylamino)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

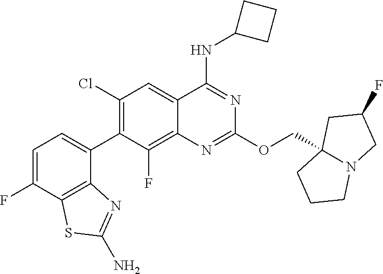 [SMILES: Nc1nc2c(-c3c(Cl)cc4c(NC5CCC5)nc(OC[C@@]56CCCN5C[C@H](F)C6)nc4c3F)ccc(F)c2s1]

Example 31 was prepared by similar procedure as described in Example 28 by replacing cyclopropanamine with cyclobutanamine to give the title product. ¹H NMR (500 MHz, DMSO-d₆) δ 10.85 (s, 1H), 8.83-8.75 (m, 1H), 8.44 (s, 1H), 7.90 (s, 2H), 7.24-7.21 (m, 1H), 7.08-7.05 (m, 1H), 5.65-5.45 (m, 1H), 4.73-4.68 (m, 1H), 4.62-4.47 (m, 2H), 3.90-3.74 (m, 3H), 2.48-2.45 (m, 1H), 2.36-2.31 (m, 3H), 2.19-2.17 (m, 4H), 2.04-2.02 (m, 1H), 1.84-1.72 (m, 2H). MS (ESI) m/e [M+1]⁺ =575.4.

### Example 32: 4-(4-(bicyclo[1.1.1]pentan-1-ylamino)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

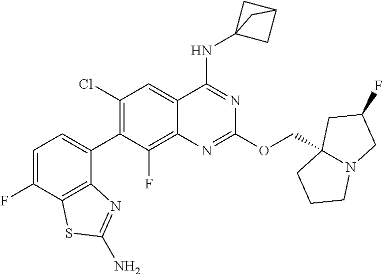 [SMILES: Nc1nc2c(-c3c(Cl)cc4c(NC56CC(C5)C6)nc(OC[C@@]56CCCN5C[C@H](F)C6)nc4c3F)ccc(F)c2s1]

Example 32 was prepared by similar procedure as described in Example 28 by replacing cyclopropanamine with bicyclo[1.1.1]pentan-1-amine to give the title product. ¹H NMR (500 MHz, DMSO-d₆) δ 9.10 (s, 1H), 8.33 (s, 1H), 7.91 (s, 2H), 7.28-7.21 (m, 1H), 7.08-7.00 (m, 1H), 5.56-5.28 (m, 1H), 4.50-4.00 (m, 2H), 3.75-3.40 (m, 1H), 3.20-2.85 (m, 3H), 2.59 (s, 1H), 2.32-1.80 (m, 12H). MS (ESI, m/e) [M+H]⁺ 587.4.

### Example 33: 4-(4-(azetidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

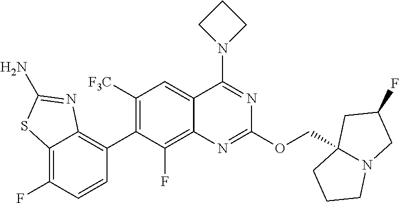 [SMILES: Nc1nc2c(-c3c(C(F)(F)F)cc4c(N5CCC5)nc(OC[C@@]56CCCN5C[C@H](F)C6)nc4c3F)ccc(F)c2s1]

Example 33 was prepared by similar procedure as described in Example 2 by replacing (1S,2R)-2-fluorocyclopropan-1-amine with azetidine to give the title product. ¹H NMR (500 MHz, CD₃OD) δ 8.07 (s, 1H), 7.20-7.13 (m, 1H), 6.98-6.91 (m, 1H), 5.43-5.27 (m, 1H), 4.79-4.52 (m, 2H), 4.43-4.25 (m, 2H), 3.54-3.34 (m, 5H), 3.18-3.09 (m, 1H), 2.67-2.56 (m, 2H), 2.44-1.87 (m, 6H). MS (ESI, m/e) [M+H]⁺ 595.4.

### Example 34: 7-fluoro-4-(8-fluoro-2-(((2R,7aS)-2-fluorotetrahydro-1H-pyrrolizin-7a(5H)-yl)methoxy)-4-(3-methylazetidin-1-yl)-6-(trifluoromethyl)quinazolin-7-yl)benzo[d]thiazol-2-amine

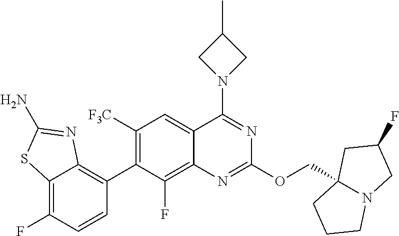 [SMILES: CC1CN(c2nc(OC[C@@]34CCCN3C[C@H](F)C4)nc3c(F)c(-c4ccc(F)c5sc(N)nc45)c(C(F)(F)F)cc23)C1]

Example 34 was prepared by similar procedure as described in Example 2 by replacing (1S,2R)-2-fluorocyclopropan-1-amine with 3-methylazetidine to give the title product. ¹H NMR (500 MHz, CD₃OD) δ 8.08 (s, 1H), 7.20-7.12 (m, 1H), 6.98-6.91 (m, 1H), 5.47-5.25 (m, 1H), 4.40-4.25 (m, 2H), 3.49-3.35 (m, 3H), 3.18-2.98 (m, 3H), 2.41-1.86 (m, 6H), 1.43-1.38 (m, 3H). MS (ESI, m/e) [M+H]⁺ 609.4.

### Example 35: 7-fluoro-4-(8-fluoro-2-(((2R,7aS)-2-fluorotetrahydro-1H-pyrrolizin-7a(5H)-yl) methoxy)-4-(2-methylazetidin-1-yl)-6-(trifluoromethyl))quinazolin-7-yl) benzo[d]thiazol-2-amine

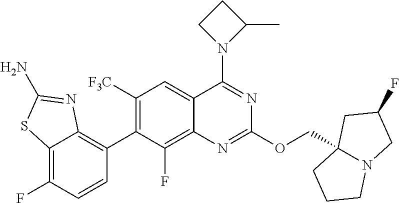 [SMILES: CC1CCN1c1nc(OC[C@@]23CCCN2C[C@H](F)C3)nc2c(F)c(-c3ccc(F)c4sc(N)nc34)c(C(F)(F)F)cc12]

Example 35 was prepared by similar procedure as described in Example 1 by replacing cyclopropanamine with 2-methylazetidine to give the title product. ¹H NMR (500 MHz, CD₃OD) δ 8.11 (s, 1H), 7.25-7.15 (m, 1H), 7.03-6.93 (m, 1H), 5.61-5.47 (m, 1H), 5.10-5.00 (m, 1H), 4.77-4.53 (m, 3H), 4.03-3.79 (m, 3H), 3.48-3.40 (m, 1H), 2.88-2.76 (m, 1H), 2.71-2.06 (m, 7H), 1.74-1.66 (m, 3H). MS (ESI, m/e) [M+H]⁺ 609.4.

### Example 36: 4-(4-(2,2-dimethylazetidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

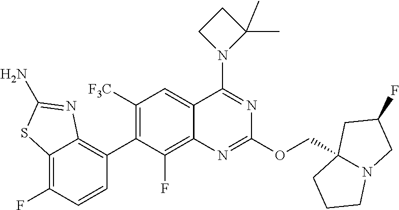 [SMILES: CC1(C)CCN1c1nc(OC[C@@]23CCCN2C[C@H](F)C3)nc2c(F)c(-c3ccc(F)c4sc(N)nc34)c(C(F)(F)F)cc12]

Example 36 was prepared by similar procedure as described in Example 1 by replacing cyclopropanamine with 2,2-dimethylazetidine to give the title product. ¹H NMR (500 MHz, CD₃OD) δ 8.13 (s, 1H), 7.19-7.14 (m, 1H), 6.98-6.91 (m, 1H), 5.42-5.26 (m, 1H), 4.85-4.76 (m, 3H), 4.37-4.21 (m, 2H), 3.51-3.34 (m, 3H), 3.15-3.06 (m, 1H), 2.45-2.38 (m, 2H), 2.36-1.87 (m, 5H), 1.82 (s, 6H). MS (ESI, m/e) [M+H]⁺ 623.4.

### Example 37: 7-fluoro-4-(8-fluoro-4-(3-fluoroazetidin-1-yl)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

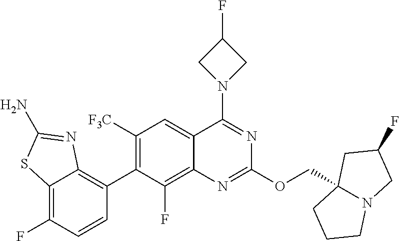 [SMILES: Nc1nc2c(-c3c(C(F)(F)F)cc4c(N5CC(F)C5)nc(OC[C@@]56CCCN5C[C@H](F)C6)nc4c3F)ccc(F)c2s1]

Example 37 was prepared by similar procedure as described in Example 2 by replacing (1S,2R)-2-fluorocyclopropan-1-amine with 3-fluoroazetidine to give the title product. ¹H NMR (500 MHz, CD₃OD) δ 8.07 (s, 1H), 7.23-7.14 (m, 1H), 7.02-6.92 (m, 1H), 5.69-5.43 (m, 2H), 5.15-4.92 (m, 2H), 4.80-4.54 (m, 4H), 3.98-3.78 (m, 3H), 3.46-3.37 (m, 1H), 2.64-2.03 (m, 6H). MS (ESI, m/e) [M+H]⁺ 708.4.

### Example 38: 4-(4-(3,3-difluoroazetidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

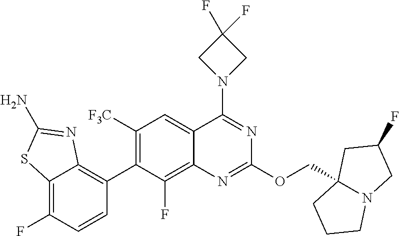 [SMILES: Nc1nc2c(-c3c(C(F)(F)F)cc4c(N5CC(F)(F)C5)nc(OC[C@@]56CCCN5C[C@H](F)C6)nc4c3F)ccc(F)c2s1]

Example 38 was prepared by similar procedure as described in Example 2 by replacing (1S,2R)-2-fluorocyclopropan-1-amine with 3,3-difluoroazetidine to give the title product. ¹H NMR (500 MHz, CD₃OD) δ 8.03 (s, 1H), 7.21-7.14 (m, 1H), 6.99-6.93 (m, 1H), 5.44-5.28 (m, 1H), 5.11-4.99 (m, 4H), 4.45-4.32 (m, 2H), 3.55-3.34 (m, 3H), 3.17-3.09 (m, 1H), 2.46-1.92 (m, 6H). MS (ESI, m/e) [M+H]⁺ 631.4.

### Example 39: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azetidine-3-carbonitrile

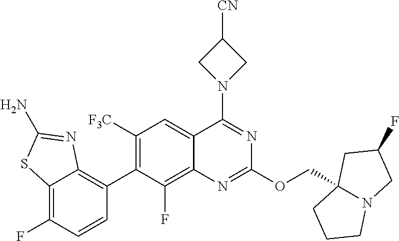 [SMILES: N#CC1CN(c2nc(OC[C@@]34CCCN3C[C@H](F)C4)nc3c(F)c(-c4ccc(F)c5sc(N)nc45)c(C(F)(F)F)cc23)C1]

Example 39 was prepared by similar procedure as described in Example 2 by replacing (1S,2R)-2-fluorocyclopropan-1-amine with azetidine-3-carbonitrile to give the title product. ¹H NMR (500 MHz, CD₃OD) δ 8.03 (s, 1H), 7.21-7.15 (m, 1H), 7.00-6.93 (m, 1H), 5.56-5.41 (m, 1H), 5.03-4.90 (m, 2H), 4.62-4.48 (m, 2H), 4.06-3.97 (m, 1H), 3.88-3.64 (m, 3H), 3.40-3.32 (m, 3H), 2.61-2.01 (m, 6H). MS (ESI, m/e) [M+H]⁺ 620.3.

### Example 40: 2-(1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azetidin-3-yl)acetonitrile

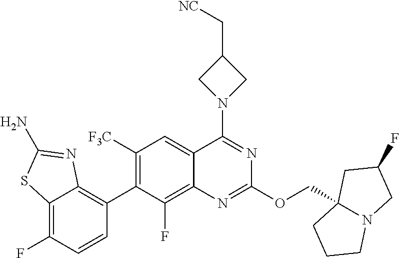 [SMILES: N#CCC1CN(c2nc(OC[C@@]34CCCN3C[C@H](F)C4)nc3c(F)c(-c4ccc(F)c5sc(N)nc45)c(C(F)(F)F)cc23)C1]

Example 40 was prepared by similar procedure as described in Example 1 by replacing cyclopropanamine with 2-(azetidin-3-yl)acetonitrile to give the title product. ¹H NMR (500 MHz, CD₃OD) δ 8.08 (s, 1H), 7.20-7.14 (m, 1H), 6.98-6.91 (m, 1H), 5.47-5.32 (m, 1H), 4.53-4.32 (m, 4H), 3.68-3.42 (m, 4H), 3.24-3.13 (m, 3H), 2.99-2.93 (m, 2H), 2.50-1.92 (m, 6H). MS (ESI, m/e) [M+H]⁺ 634.6.

### Example 41: 3-(1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azetidin-3-yl)propanenitrile

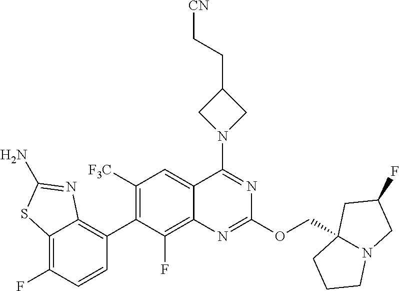 [SMILES: N#CCCC1CN(c2nc(OC[C@@]34CCCN3C[C@H](F)C4)nc3c(F)c(-c4ccc(F)c5sc(N)nc45)c(C(F)(F)F)cc23)C1]

Example 41 was prepared by similar procedure as described in Example 1 by replacing cyclopropanamine with 3-(azetidin-3-yl)propanenitrile to give the title product. ¹H NMR (500 MHz, CD₃OD) δ 8.09 (s, 1H), 7.21-7.14 (m, 1H), 7.00-6.93 (m, 1H), 5.49-5.32 (m, 1H), 4.74-4.36 (m, 4H), 3.71-3.45 (m, 3H), 3.25-3.15 (m, 3H), 3.11-3.01 (m, 1H), 2.61-2.53 (m, 2H), 2.52-1.91 (m, 8H). MS (ESI, m/e) [M+H]⁺ 648.5.

### Example 42: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methylazetidine-3-carbonitrile

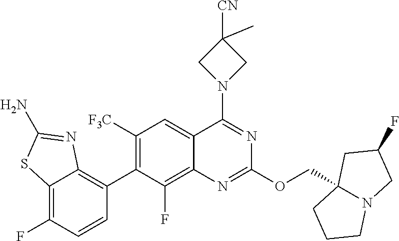 [SMILES: CC1(C#N)CN(c2nc(OC[C@@]34CCCN3C[C@H](F)C4)nc3c(F)c(-c4ccc(F)c5sc(N)nc45)c(C(F)(F)F)cc23)C1]

Example 42 was prepared by similar procedure as described in Example 1 by replacing cyclopropanamine with 3-methylazetidine-3-carbonitrile to give the title product. ¹H NMR (500 MHz, CD₃OD) δ 8.03 (s, 1H), 7.22-7.13 (m, 1H), 7.02-6.92 (m, 1H), 5.55-5.41 (m, 1H), 4.62-4.53 (m, 4H), 3.83-3.67 (m, 3H), 2.61-1.97 (m, 9H), 1.83 (s, 3H). MS (ESI, m/e) [M+H]⁺ 634.2.

### Example 43: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2-methylazetidine-3-carbonitrile

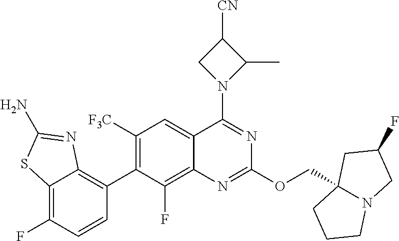 [SMILES: CC1C(C#N)CN1c1nc(OC[C@@]23CCCN2C[C@H](F)C3)nc2c(F)c(-c3ccc(F)c4sc(N)nc34)c(C(F)(F)F)cc12]

Example 43 was prepared by similar procedure as described in Example 1 by replacing cyclopropanamine with 2-methylazetidine-3-carbonitrile to give the title product. 1H NMR (500 MHz, CD₃OD) δ 8.01 (s, 1H), 7.22-7.13 (m, 1H), 7.02-6.93 (m, 1H), 5.60-5.40 (m, 1H), 5.26-5.03 (m, 2H), 4.68-4.51 (m, 2H), 4.20-4.11 (m, 1H), 3.92-3.70 (m, 3H), 3.43-3.35 (m, 1H), 2.66-2.02 (m, 6H), 1.87-1.78 (m, 3H). MS (ESI, m/e) [M+H]⁺ 634.4.

### Example 44: 7-fluoro-4-(8-fluoro-2-(((2R,7aS)-2-fluorotetrahydro-1H-pyrrolizin-7a(5H)-yl)methoxy)-4-(3-(methylsulfonyl)azetidin-1-yl)-6-(trifluoromethyl)quinazolin-7-yl)benzo[d]thiazol-2-amine

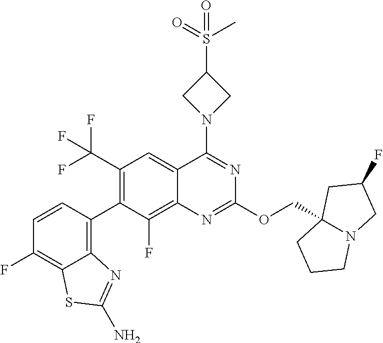 [SMILES: CS(=O)(=O)C1CN(c2nc(OC[C@@]34CCCN3C[C@H](F)C4)nc3c(F)c(-c4ccc(F)c5sc(N)nc45)c(C(F)(F)F)cc23)C1]

Example 44 was prepared by similar procedure as described in Example 1 by replacing cyclopropanamine with 3-(methylsulfonyl)azetidine to give the title product. 1H NMR (500 MHz, CD₃OD) δ 8.07 (s, 1H), 7.24-7.14 (m, 1H), 7.01-6.92 (m, 1H), 5.59-5.42 (m, 1H), 4.66-4.48 (m, 3H), 3.93-3.54 (m, 3H), 3.47-3.35 (m, 3H), 3.10 (s, 1H), 2.90-2.85 (m, 1H), 2.70-2.10 (m, 6H). MS (ESI, m/e) [M+H]⁺ 673.5.

### Example 45: 7-fluoro-4-(8-fluoro-2-(((2R,7aS)-2-fluorotetrahydro-1H-pyrrolizin-7a(5H)-yl)methoxy)-4-(3-methoxyazetidin-1-yl)-6-(trifluoromethyl)quinazolin-7-yl)benzo[d]thiazol-2-amine

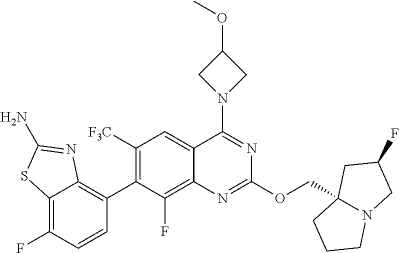 [SMILES: COC1CN(c2nc(OC[C@@]34CCCN3C[C@H](F)C4)nc3c(F)c(-c4ccc(F)c5sc(N)nc45)c(C(F)(F)F)cc23)C1]

Example 45 was prepared by similar procedure as described in Example 1 by replacing cyclopropanamine with 3-methoxyazetidine to give the title product. ¹H NMR (500 MHz, CD₃OD) δ 8.09 (s, 1H), 7.22-7.12 (m, 1H), 7.02-6.91 (m, 1H), 5.51-5.32 (m, 1H), 4.62-4.23 (m, 5H), 3.75-3.46 (m, 3H), 3.41 (s, 3H), 3.26-3.15 (m, 2H), 2.56-1.90 (m, 6H). MS (ESI, m/e) [M+H]⁺ 625.4.

### Example 46: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methylazetidin-3-ol

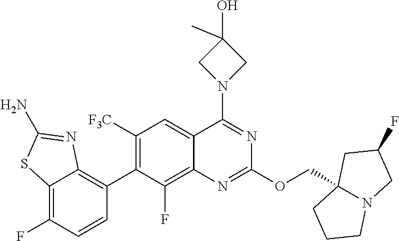 [SMILES: CC1(O)CN(c2nc(OC[C@@]34CCCN3C[C@H](F)C4)nc3c(F)c(-c4ccc(F)c5sc(N)nc45)c(C(F)(F)F)cc23)C1]

Example 46 was prepared by similar procedure as described in Example 1 by replacing cyclopropanamine with 3-methylazetidin-3-ol to give the title product. ¹H NMR (500 MHz, CD₃OD) δ 8.07 (s, 1H), 7.22-7.12 (m, 1H), 7.02-6.91 (m, 1H), 5.45-5.27 (m, 1H), 4.75-4.25 (m, 6H), 3.54-3.35 (m, 3H), 3.20-3.05 (m, 1H), 2.47-1.80 (m, 6H), 1.61 (s, 3H). MS (ESI, m/e) [M+H]⁺ 625.4.

### Example 47: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2-methylazetidin-3-ol

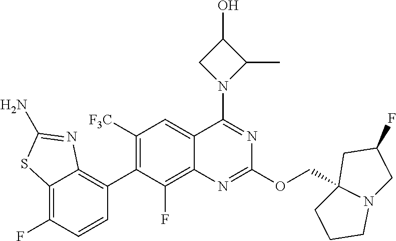 [SMILES: CC1C(O)CN1c1nc(OC[C@@]23CCCN2C[C@H](F)C3)nc2c(F)c(-c3ccc(F)c4sc(N)nc34)c(C(F)(F)F)cc12]

Example 47 was prepared by similar procedure as described in Example 1 by replacing cyclopropanamine with 2-methylazetidin-3-ol to give the title product. ¹H NMR (500 MHz, CD₃OD) δ 8.06 (s, 1H), 7.25-7.09 (m, 1H), 7.02-6.91 (m, 1H), 5.45-5.28 (m, 1H), 5.22-5.03 (m, 1H), 4.63-4.24 (m, 3H), 3.55-3.34 (m, 3H), 3.20-3.06 (m, 1H), 2.48-1.87 (m, 6H), 1.70-1.54 (m, 3H). MS (ESI, m/e) [M+H]⁺ 625.4.

### Example 48: (1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azetidin-3-yl)methanol

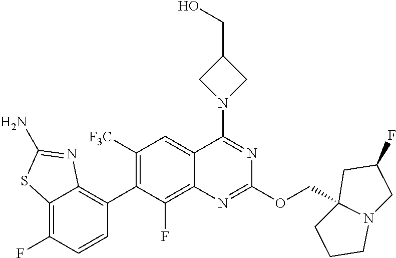 [SMILES: Nc1nc2c(-c3c(C(F)(F)F)cc4c(N5CC(CO)C5)nc(OC[C@@]56CCCN5C[C@H](F)C6)nc4c3F)ccc(F)c2s1]

Example 48 was prepared by similar procedure as described in Example 1 by replacing cyclopropanamine with azetidin-3-ylmethanol to give the title product. ¹H NMR (500 MHz, CD₃OD) δ 8.11 (s, 1H), 7.30-7.12 (m, 1H), 7.02-6.91 (m, 1H), 5.48-5.28 (m, 1H), 4.73-4.25 (m, 5H), 3.88-3.75 (m, 2H), 3.67-3.37 (m, 4H), 3.23-3.00 (m, 2H), 2.48-1.89 (m, 6H). MS (ESI, m/e) [M+H]⁺ 625.4.

### Example 49: ((2R)-1-(7-(2-amino-7-fluorobenzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl) azetidin-2-yl) methanol

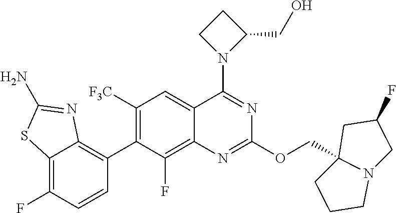 [SMILES: Nc1nc2c(-c3c(C(F)(F)F)cc4c(N5CC[C@@H]5CO)nc(OC[C@@]56CCCN5C[C@H](F)C6)nc4c3F)ccc(F)c2s1]

Example 49 was prepared by similar procedure as described in Example 1 by replacing cyclopropanamine with (R)-azetidin-2-ylmethanol to give the title product. ¹H NMR (500 MHz, CD₃OD) δ 8.12 (s, 1H), 7.22-7.12 (m, 1H), 7.01-6.91 (m, 1H), 5.49-5.34 (m, 1H), 5.04-4.96 (m, 1H), 4.83-4.76 (m, 1H), 4.72-4.64 (m, 1H), 4.48-4.40 (m, 2H), 4.24-4.17 (m, 1H), 3.88-3.84 (m, 1H), 3.69-3.43 (m, 3H), 3.25-3.20 (m, 1H), 2.70-1.94 (m, 8H). MS (ESI, m/e) [M+H]⁺ 625.5.

### Example 50: 7-fluoro-4-(8-fluoro-2-(((2R,7aS)-2-fluorotetrahydro-1H-pyrrolizin-7a(5H)-yl)methoxy)-4-(pyrrolidin-1-yl)-6-(trifluoromethyl)quinazolin-7-yl)benzo[d]thiazol-2-amine

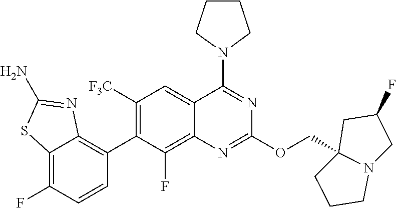 [SMILES: Nc1nc2c(-c3c(C(F)(F)F)cc4c(N5CCCC5)nc(OC[C@@]56CCCN5C[C@H](F)C6)nc4c3F)ccc(F)c2s1]

Example 50 was prepared by similar procedure as described in Example 2 by replacing (1S,2R)-2-fluorocyclopropan-1-amine with pyrrolidine to give the title product. ¹H NMR (500 MHz, CD₃OD) δ 8.47 (s, 1H), 7.22-7.14 (m, 1H), 6.99-6.92 (m, 1H), 5.47-5.30 (m, 1H), 4.48-4.32 (m, 2H), 4.09-3.98 (m, 4H), 3.62-3.39 (m, 3H), 3.22-3.12 (m, 1H), 2.50-1.91 (m, 10H). MS (ESI, m/e) [M+H]⁺ 609.4.

### Example 51: 7-fluoro-4-(8-fluoro-4-((R)-3-fluoropyrrolidin-1-yl)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

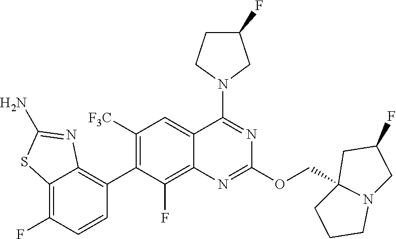 [SMILES: Nc1nc2c(-c3c(C(F)(F)F)cc4c(N5CC[C@@H](F)C5)nc(OC[C@@]56CCCN5C[C@H](F)C6)nc4c3F)ccc(F)c2s1]

Example 51 was prepared by similar procedure as described in Example 2 by replacing (1S,2R)-2-fluorocyclopropan-1-amine with (R)-3-fluoropyrrolidine to give the title product. ¹H NMR (500 MHz, CD₃OD) δ 8.44 (s, 1H), 7.21-7.15 (m, 1H), 7.00-6.91 (m, 1H), 5.55-5.25 (m, 2H), 4.41-4.01 (m, 6H), 3.49-3.40 (m, 1H), 3.10-3.02 (m, 1H), 2.58-1.83 (m, 8H). MS (ESI, m/e) [M+H]⁺ 627.4.

### Example 52: 7-fluoro-4-(8-fluoro-4-((S)-3-fluoropyrrolidin-1-yl)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

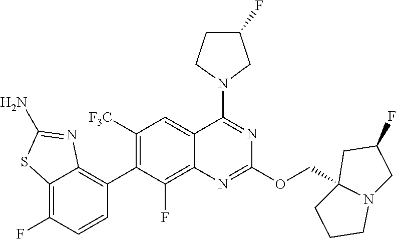 [SMILES: Nc1nc2c(-c3c(C(F)(F)F)cc4c(N5CC[C@H](F)C5)nc(OC[C@@]56CCCN5C[C@H](F)C6)nc4c3F)ccc(F)c2s1]

Example 52 was prepared by similar procedure as described in Example 2 by replacing (1S,2R)-2-fluorocyclopropan-1-amine with (S)-3-fluoropyrrolidine to give the title product. ¹H NMR (500 MHz, CD₃OD) δ 8.48 (s, 1H), 7.23-7.15 (m, 1H), 7.02-6.94 (m, 1H), 5.57-5.41 (m, 2H), 4.68-4.53 (m, 2H), 4.36-4.19 (m, 4H), 3.98-3.68 (m, 3H), 3.43-3.35 (m, 1H), 2.66-2.02 (m, 8H). MS (ESI, m/e) [M+H]⁺ 627.4.

### Example 53: 4-(4-(3,3-difluoropyrrolidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

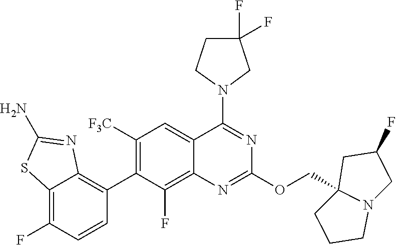 [SMILES: Nc1nc2c(-c3c(C(F)(F)F)cc4c(N5CCC(F)(F)C5)nc(OC[C@@]56CCCN5C[C@H](F)C6)nc4c3F)ccc(F)c2s1]

Example 53 was prepared by similar procedure as described in Example 2 by replacing (1S,2R)-2-fluorocyclopropan-1-amine with 3,3-difluoropyrrolidine to give the title product. ¹H NMR (500 MHz, CD₃OD) δ 8.38 (s, 1H), 7.22-7.15 (m, 1H), 6.99-6.93 (m, 1H), 5.47-5.31 (m, 1H), 4.50-4.31 (m, 6H), 3.61-3.38 (m, 3H), 3.21-3.12 (m, 1H), 2.70-2.58 (m, 2H), 2.50-1.93 (m, 6H). MS (ESI, m/e) [M+H]⁺ 645.4.

### Example 54: 7-fluoro-4-(8-fluoro-2-(((2R,7aS)-2-fluorotetrahydro-1H-pyrrolizin-7a(5H)-yl)methoxy)-4-((R)-3-methylpyrrolidin-1-yl)-6-(trifluoromethyl)quinazolin-7-yl)benzo[d]thiazol-2-amine

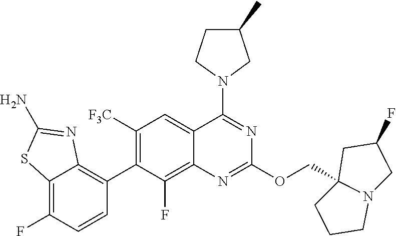 [SMILES: C[C@@H]1CCN(c2nc(OC[C@@]34CCCN3C[C@H](F)C4)nc3c(F)c(-c4ccc(F)c5sc(N)nc45)c(C(F)(F)F)cc23)C1]

Example 54 was prepared by similar procedure as described in Example 1 by replacing cyclopropanamine with (R)-3-methylpyrrolidine to give the title product. ¹H NMR (500 MHz, CD₃OD) δ 8.44 (s, 1H), 7.21-7.15 (m, 1H), 6.98-6.91 (m, 1H), 5.42-5.27 (m, 1H), 4.42-4.25 (m, 2H), 4.23-3.98 (m, 3H), 3.65-3.34 (m, 3H), 3.14-3.03 (m, 1H), 2.55-1.68 (m, 10H), 1.24-1.18 (m, 3H). MS (ESI, m/e) [M+H]⁺ 623.5.

### Example 55: 7-fluoro-4-(8-fluoro-2-(((2R,7aS)-2-fluorotetrahydro-1H-pyrrolizin-7a(5H)-yl)methoxy)-4-((R)-2-methylpyrrolidin-1-yl)-6-(trifluoromethyl)quinazolin-7-yl)benzo[d]thiazol-2-amine

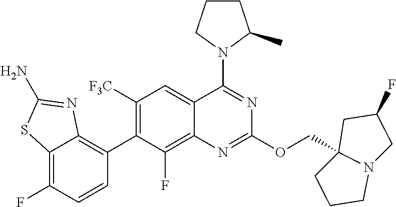 [SMILES: C[C@@H]1CCCN1c1nc(OC[C@@]23CCCN2C[C@H](F)C3)nc2c(F)c(-c3ccc(F)c4sc(N)nc34)c(C(F)(F)F)cc12]

Example 55 was prepared by similar procedure as described in Example 1 by replacing cyclopropanamine with (R)-2-methylpyrrolidine to give the title product. ¹H NMR (500 MHz, CD₃OD) δ 8.46 (s, 1H), 7.24-7.13 (m, 1H), 7.02-6.91 (m, 1H), 5.65-5.43 (m, 1H), 4.87-4.57 (m, 3H), 4.31-3.82 (m, 5H), 3.49-3.42 (m, 1H), 2.74-1.81 (m, 10H), 1.52-1.45 (m, 3H). MS (ESI, m/e) [M+H]⁺ 623.5.

### Example 56: 7-fluoro-4-(8-fluoro-2-(((2R,7aS)-2-fluorotetrahydro-1H-pyrrolizin-7a(5H)-yl)methoxy)-4-((S)-2-methylpyrrolidin-1-yl)-6-(trifluoromethyl)quinazolin-7-yl)benzo[d]thiazol-2-amine

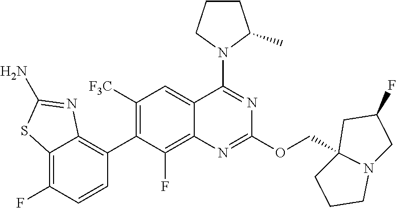 [SMILES: C[C@H]1CCCN1c1nc(OC[C@@]23CCCN2C[C@H](F)C3)nc2c(F)c(-c3ccc(F)c4sc(N)nc34)c(C(F)(F)F)cc12]

Example 56 was prepared by similar procedure as described in Example 1 by replacing cyclopropanamine with (S)-2-methylpyrrolidine to give the title product. ¹H NMR (500 MHz, CD₃OD) δ 8.47 (s, 1H), 7.28-7.14 (m, 1H), 7.03-6.93 (m, 1H), 5.60-5.43 (m, 1H), 4.86-4.68 (m, 2H), 4.30-3.85 (m, 5H), 3.48-3.43 (m, 1H), 2.72-1.85 (m, 10H), 1.52-1.45 (m, 3H). MS (ESI, m/e) [M+H]⁺ 623.5.

### Example 57: 4-(4-(2,2-dimethylpyrrolidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

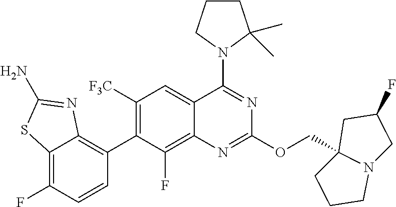 [SMILES: CC1(C)CCCN1c1nc(OC[C@@]23CCCN2C[C@H](F)C3)nc2c(F)c(-c3ccc(F)c4sc(N)nc34)c(C(F)(F)F)cc12]

Example 57 was prepared by similar procedure as described in Example 2 by replacing (1S,2R)-2-fluorocyclopropan-1-amine with 2,2-dimethylpyrrolidine to give the title product. ¹H NMR (500 MHz, CD₃OD) δ 8.45 (s, 1H), 7.21-7.15 (m, 1H), 6.98-6.91 (m, 1H), 5.41-5.25 (m, 1H), 4.40-4.26 (m, 2H), 4.26-4.20 (m, 2H), 3.23-3.01 (m, 4H), 2.43-1.84 (m, 10H), 1.79 (s, 6H). MS (ESI, m/e) [M+H]⁺ 636.9.

### Example 58: 4-(4-((R)-2-ethylpyrrolidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

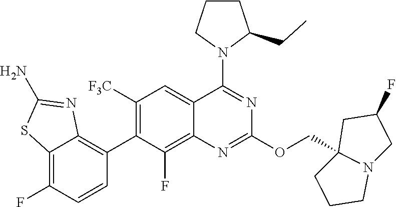 [SMILES: CC[C@@H]1CCCN1c1nc(OC[C@@]23CCCN2C[C@H](F)C3)nc2c(F)c(-c3ccc(F)c4sc(N)nc34)c(C(F)(F)F)cc12]

Example 58 was prepared by similar procedure as described in Example 1 by replacing cyclopropanamine with (R)-2-ethylpyrrolidine to give the title product. ¹H NMR (500 MHz, CD₃OD) δ 8.39 (s, 1H), 7.25-7.12 (m, 1H), 7.02-6.91 (m, 1H), 5.55-5.35 (m, 1H), 4.70-4.42 (m, 3H), 4.24-4.05 (m, 2H), 3.83-3.55 (m, 3H), 2.63-2.41 (m, 2H), 2.33-1.89 (m, 10H), 1.72-1.58 (m, 1H), 1.09-1.00 (m, 3H). MS (ESI, m/e) [M+H]⁺ 637.5.

### Example 59: 4-(4-((S)-2-ethylpyrrolidin-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

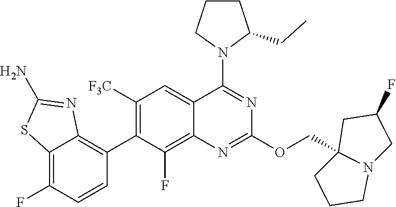 [SMILES: CC[C@H]1CCCN1c1nc(OC[C@@]23CCCN2C[C@H](F)C3)nc2c(F)c(-c3ccc(F)c4sc(N)nc34)c(C(F)(F)F)cc12]

Example 59 was prepared by similar procedure as described in Example 1 by replacing cyclopropanamine with (S)-2-ethylpyrrolidine to give the title product. ¹H NMR (500 MHz, CD₃OD) δ 8.41 (s, 1H), 7.25-7.14 (m, 1H), 7.02-6.93 (m, 1H), 5.62-5.44 (m, 1H), 4.70-4.60 (m, 3H), 4.22-4.07 (m, 2H), 4.04-3.81 (m, 3H), 3.47-3.43 (m, 1H), 2.70-2.53 (m, 2H), 2.42-1.92 (m, 10H), 1.72-1.57 (m, 1H), 1.09-1.00 (m, 3H). MS (ESI, m/e) [M+H]⁺ 637.5.

### Example 60: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidine-3-carbonitrile

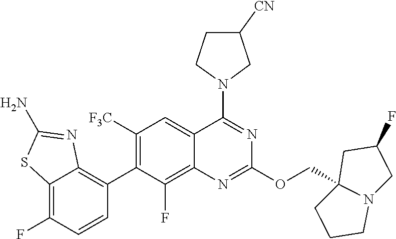 [SMILES: N#CC1CCN(c2nc(OC[C@@]34CCCN3C[C@H](F)C4)nc3c(F)c(-c4ccc(F)c5sc(N)nc45)c(C(F)(F)F)cc23)C1]

Example 60 was prepared by similar procedure as described in Example 2 by replacing (1S,2R)-2-fluorocyclopropan-1-amine with pyrrolidine-3-carbonitrile to give the title product. ¹H NMR (500 MHz, CD₃OD) δ 8.40 (s, 1H), 7.21-7.14 (m, 1H), 6.99-6.92 (m, 1H), 5.42-5.25 (m, 1H), 4.44-4.15 (m, 6H), 3.63-3.54 (m, 1H), 3.15-3.04 (m, 1H), 2.59-1.85 (m, 8H). MS (ESI, m/e) [M+H]⁺ 634.4.

### Example 61: 2-(1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidin-3-yl)acetonitrile

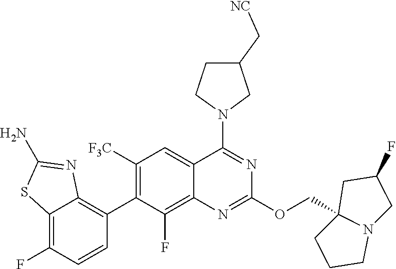 [SMILES: N#CCC1CCN(c2nc(OC[C@@]34CCCN3C[C@H](F)C4)nc3c(F)c(-c4ccc(F)c5sc(N)nc45)c(C(F)(F)F)cc23)C1]

Example 61 was prepared by similar procedure as described in Example 2 by replacing (1S,2R)-2-fluorocyclopropan-1-amine with 2-(pyrrolidine-3-yl)acetonitrile to give the title product. ¹H NMR (500 MHz, CD₃OD) δ 8.43 (s, 1H), 7.21-7.15 (m, 1H), 6.98-6.91 (m, 1H), 5.41-5.27 (m, 1H), 4.43-4.07 (m, 5H), 3.85-3.77 (m, 1H), 3.47-3.36 (m, 3H), 3.13-3.03 (m, 1H), 2.85-2.72 (m, 3H), 2.45-1.86 (m, 8H). MS (ESI, m/e) [M+H]⁺ 647.8.

### Example 62: 4-(4-(2-azabicyclo[2.1.1]hexan-2-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

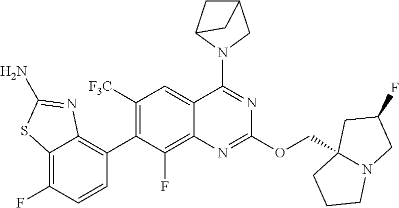 [SMILES: Nc1nc2c(-c3c(C(F)(F)F)cc4c(N5CC6CC5C6)nc(OC[C@@]56CCCN5C[C@H](F)C6)nc4c3F)ccc(F)c2s1]

Example 62 was prepared by similar procedure as described in Example 1 by replacing cyclopropanamine with 2-azabicyclo[2.1.1]hexane to give the title product. ¹H NMR (500 MHz, CD₃OD) δ 8.41 (s, 1H), 7.23-7.14 (m, 1H), 7.01-6.91 (m, 1H), 5.50-5.29 (m, 2H), 4.42-4.27 (m, 2H), 4.21-4.09 (m, 2H), 3.56-3.39 (m, 3H), 3.19-3.10 (m, 2H), 2.47-1.93 (m, 8H), 1.62-1.53 (m, 2H). MS (ESI, m/e) [M+H]⁺ 621.4.

### Example 63: (3R)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidin-3-ol

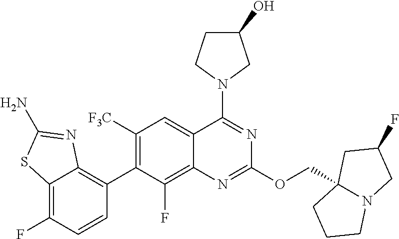 [SMILES: Nc1nc2c(-c3c(C(F)(F)F)cc4c(N5CC[C@@H](O)C5)nc(OC[C@@]56CCCN5C[C@H](F)C6)nc4c3F)ccc(F)c2s1]

Example 63 was prepared by similar procedure as described in Example 1 by replacing cyclopropanamine with (R)-pyrrolidin-3-ol to give the title product. ¹H NMR (500 MHz, CD₃OD) δ 8.46 (s, 1H), 7.25-7.13 (m, 1H), 7.01-6.90 (m, 1H), 5.50-5.32 (m, 1H), 4.65-4.37 (m, 3H), 4.32-4.03 (m, 3H), 3.99-3.91 (m, 1H), 3.75-3.40 (m, 3H), 3.27-3.12 (m, 1H), 2.60-1.88 (m, 8H). MS (ESI, m/e) [M+H]⁺ 625.3.

### Example 64: (3S)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidin-3-ol

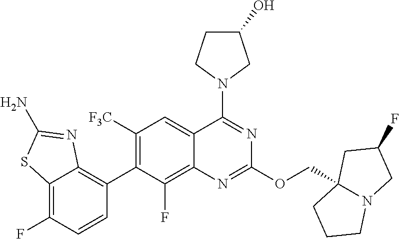 [SMILES: Nc1nc2c(-c3c(C(F)(F)F)cc4c(N5CC[C@H](O)C5)nc(OC[C@@]56CCCN5C[C@H](F)C6)nc4c3F)ccc(F)c2s1]

Example 64 was prepared by similar procedure as described in Example 1 by replacing cyclopropanamine with (S)-pyrrolidin-3-ol to give the title product. ¹H NMR (500 MHz, CD₃OD) δ 8.45 (s, 1H), 7.25-7.11 (m, 1H), 7.01-6.90 (m, 1H), 5.48-5.32 (m, 1H), 4.65-4.33 (m, 3H), 4.31-4.02 (m, 3H), 3.99-3.91 (m, 1H), 3.62-3.37 (m, 3H), 3.21-3.07 (m, 1H), 2.54-1.89 (m, 8H). MS (ESI, m/e) [M+H]⁺ 625.4.

### Example 65: ((3R)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidin-3-yl)methanol

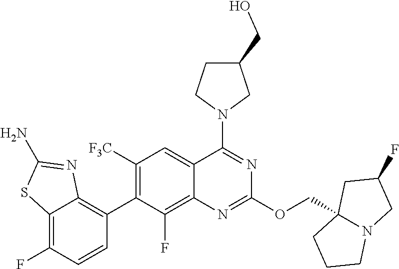 [SMILES: Nc1nc2c(-c3c(C(F)(F)F)cc4c(N5CC[C@@H](CO)C5)nc(OC[C@@]56CCCN5C[C@H](F)C6)nc4c3F)ccc(F)c2s1]

Example 65 was prepared by similar procedure as described in Example 1 by replacing cyclopropanamine with (R)-pyrrolidin-3-ylmethanol to give the title product. ¹H NMR (500 MHz, CD₃OD) δ 8.46 (s, 1H), 7.23-7.13 (m, 1H), 7.01-6.90 (m, 1H), 5.47-5.30 (m, 1H), 4.47-4.30 (m, 2H), 4.27-3.80 (m, 4H), 3.76-3.60 (m, 2H), 3.57-3.33 (m, 3H), 3.18-3.10 (m, 1H), 2.68-2.54 (m, 1H), 2.48-1.87 (m, 8H). MS (ESI, m/e) [M+H]⁺ 639.5.

### Example 66: ((3S)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)pyrrolidin-3-yl)methanol

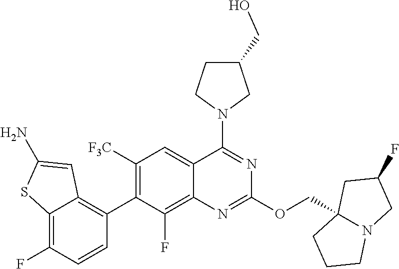 [SMILES: Nc1cc2c(-c3c(C(F)(F)F)cc4c(N5CC[C@H](CO)C5)nc(OC[C@@]56CCCN5C[C@H](F)C6)nc4c3F)ccc(F)c2s1]

Example 66 was prepared by similar procedure as described in Example 1 by replacing cyclopropanamine with (S)-pyrrolidin-3-ylmethanol to give the title product. ¹H NMR (500 MHz, CD₃OD) δ 8.46 (s, 1H), 7.25-7.09 (m, 1H), 7.01-6.90 (m, 1H), 5.47-5.30 (m, 1H), 4.48-4.29 (m, 2H), 4.24-3.80 (m, 4H), 3.76-3.60 (m, 2H), 3.57-3.34 (m, 3H), 3.20-3.06 (m, 1H), 2.69-2.56 (m, 1H), 2.49-1.85 (m, 8H). MS (ESI, m/e) [M+H]⁺ 639.5.

### Example 67: 7-fluoro-4-(8-fluoro-2-(((2R,7aS)-2-fluorotetrahydro-1H-pyrrolizin-7a(5H)-yl)methoxy)-4-(piperidin-1-yl)-6-(trifluoromethyl)quinazolin-7-yl)benzo[d]thiazol-2-amine

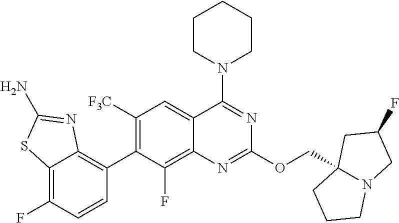 [SMILES: Nc1nc2c(-c3c(C(F)(F)F)cc4c(N5CCCCC5)nc(OC[C@@]56CCCN5C[C@H](F)C6)nc4c3F)ccc(F)c2s1]

Example 67 was prepared by similar procedure as described in Example 2 by replacing (1S,2R)-2-fluorocyclopropan-1-amine with piperidine to give the title product. 1H NMR (500 MHz, CD₃OD) δ 8.12 (s, 1H), 7.22-7.15 (m, 1H), 6.99-6.93 (m, 1H), 5.46-5.30 (m, 1H), 4.46-4.27 (m, 2H), 4.00-3.89 (m, 4H), 3.56-3.35 (m, 3H), 3.19-3.10 (m, 1H), 2.48-1.90 (m, 6H), 1.88-1.79 (m, 6H). MS (ESI, m/e) [M+H]⁺ 623.4.

### Example 68: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carbonitrile

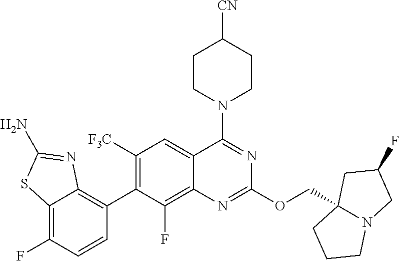 [SMILES: N#CC1CCN(c2nc(OC[C@@]34CCCN3C[C@H](F)C4)nc3c(F)c(-c4ccc(F)c5sc(N)nc45)c(C(F)(F)F)cc23)CC1]

Example 68 was prepared by similar procedure as described in Example 2 by replacing (1S,2R)-2-fluorocyclopropan-1-amine with piperidine-4-carbonitrile to give the title product. 1H NMR (500 MHz, CD₃OD) δ 8.10 (s, 1H), 7.21-7.14 (m, 1H), 7.00-6.93 (m, 1H), 5.37-5.21 (m, 1H), 4.35-4.14 (m, 4H), 3.85-3.75 (m, 2H), 3.26-3.14 (m, 4H), 3.04-2.96 (m, 1H), 2.35-1.85 (m, 10H). MS (ESI, m/e) [M+H]⁺ 648.4.

### Example 69: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-4-methylpiperidine-4-carbonitrile

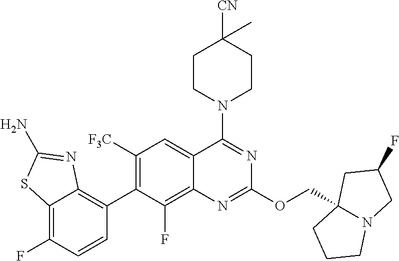

Example 69 was prepared by similar procedure as described in Example 1 by replacing cyclopropanamine with 4-methylpiperidine-4-carbonitrile to give the title product. ¹H NMR (500 MHz, CD₃OD) δ 8.12 (s, 1H), 7.23-7.13 (m, 1H), 7.00-6.92 (m, 1H), 5.40-5.21 (m, 1H), 4.56-4.47 (m, 2H), 4.36-4.22 (m, 2H), 3.70-3.54 (m, 2H), 3.28-3.15 (m, 3H), 3.08-2.97 (m, 1H), 2.39-1.79 (m, 10H), 1.49 (s, 3H). MS (ESI, m/e) [M+H]⁺ 662.6.

### Example 70: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-4-fluoropiperidine-4-carbonitrile

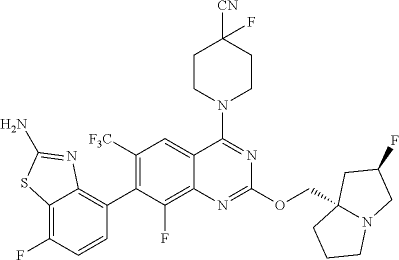 [SMILES: N#CC1(F)CCN(c2nc(OC[C@@]34CCCN3C[C@H](F)C4)nc3c(F)c(-c4ccc(F)c5sc(N)nc45)c(C(F)(F)F)cc23)CC1]

Example 70 was prepared by similar procedure as described in Example 1 by replacing cyclopropanamine with 4-fluoropiperidine-4-carbonitrile to give the title product. ¹H NMR (500 MHz, CD₃OD) δ 8.18 (s, 1H), 7.25-7.13 (m, 1H), 7.01-6.92 (m, 1H), 5.51-5.31 (m, 1H), 4.58-4.40 (m, 2H), 4.25-3.96 (m, 4H), 3.74-3.41 (m, 3H), 3.27-3.15 (m, 1H), 2.59-1.95 (m, 10H). MS (ESI, m/e) [M+H]⁺ 666.5.

### Example 71: 2-(1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidin-4-yl)acetonitrile

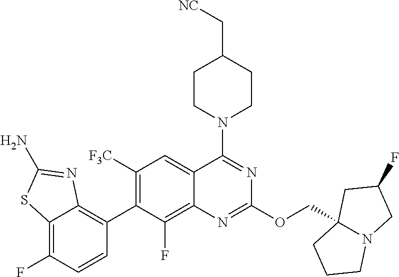

Example 71 was prepared by similar procedure as described in Example 1 by replacing cyclopropanamine with 2-(piperidin-4-yl)acetonitrile to give the title product. ¹H NMR (500 MHz, CD₃OD) δ 8.12 (s, 1H), 7.23-7.14 (m, 1H), 7.01-6.92 (m, 1H), 5.46-5.28 (m, 1H), 4.66-4.52 (m, 2H), 4.45-4.28 (m, 2H), 3.53-3.32 (m, 5H), 3.19-3.05 (m, 1H), 2.60-2.50 (m, 2H), 2.47-1.89 (m, 9H), 1.67-1.53 (m, 2H). MS (ESI, m/e) [M+H]⁺ 662.4.

### Example 72: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-fluoropiperidine-4-carbonitrile

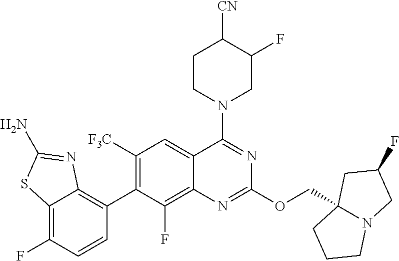 [SMILES: N#CC1CCN(c2nc(OC[C@@]34CCCN3C[C@H](F)C4)nc3c(F)c(-c4ccc(F)c5sc(N)nc45)c(C(F)(F)F)cc23)CC1F]

Example 72 was prepared by similar procedure as described in Example 1 by replacing cyclopropanamine with 3-fluoropiperidine-4-carbonitrile to give the title product. ¹H NMR (500 MHz, CD₃OD) δ 8.22 (s, 1H), 7.23-7.14 (m, 1H), 7.01-6.92 (m, 1H), 5.59-5.39 (m, 1H), 5.14-5.00 (m, 1H), 4.67-4.52 (m, 4H), 3.91-3.45 (m, 6H), 2.62-2.06 (m, 8H). MS (ESI, m/e) [M+H]⁺ 666.5.

### Example 73: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methylpiperidine-4-carbonitrile

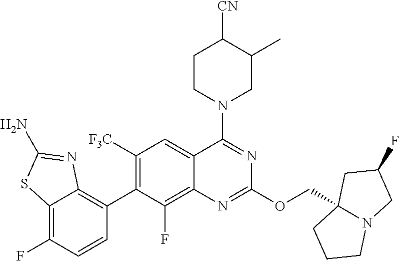 [SMILES: CC1CN(c2nc(OC[C@@]34CCCN3C[C@H](F)C4)nc3c(F)c(-c4ccc(F)c5sc(N)nc45)c(C(F)(F)F)cc23)CCC1C#N]

Example 73 was prepared by similar procedure as described in Example 1 by replacing cyclopropanamine with 3-methylpiperidine-4-carbonitrile to give the title product. ¹H NMR (500 MHz, CD₃OD) δ 8.16-8.09 (m, 1H), 7.22-7.16 (m, 1H), 7.00-6.93 (m, 1H), 5.47-5.32 (m, 1H), 4.58-4.25 (m, 3H), 3.73-3.35 (m, 6H), 3.24-3.06 (m, 2H), 2.87-2.75 (m, 1H), 2.54-1.90 (m, 8H), 1.25-1.16 (m, 3H). MS (ESI, m/e) [M+H]⁺ 662.6.

### Example 74: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-trans-2-methylpiperidine-4-carbonitrile

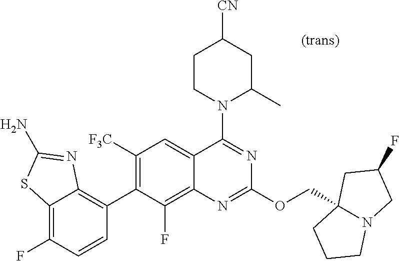 [SMILES: CC1CC(C#N)CCN1c1nc(OC[C@@]23CCCN2C[C@H](F)C3)nc2c(F)c(-c3ccc(F)c4sc(N)nc34)c(C(F)(F)F)cc12]

Example 74 was prepared by similar procedure as described in Example 1 by replacing cyclopropanamine with trans-2-methylpiperidine-4-carbonitrile to give the title product. ¹H NMR (500 MHz, CD₃OD) δ 8.07 (s, 1H), 7.22-7.16 (m, 1H), 7.00-6.93 (m, 1H), 5.48-5.32 (m, 1H), 5.00-4.92 (m, 2H), 4.55-4.36 (m, 3H), 3.68-3.46 (m, 4H), 2.44-1.85 (m, 8H), 1.59-1.51 (m, 3H). MS (ESI, m/e) [M+H]⁺ 662.5.

### Example 75: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-trans-3-hydroxypiperidine-4-carbonitrile

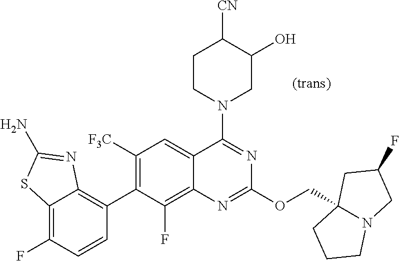

Example 75 was prepared by similar procedure as described in Example 1 by replacing cyclopropanamine with trans-3-hydroxypiperidine-4-carbonitrile to give the title product. ¹H NMR (500 MHz, CD₃OD) δ 8.26 (s, 1H), 7.25-7.13 (m, 1H), 7.02-6.93 (m, 1H), 5.49-5.32 (m, 1H), 4.55-4.34 (m, 3H), 4.26-4.95 (m, 2H), 3.78-3.41 (m, 5H), 3.26-3.14 (m, 1H), 3.02-2.90 (m, 1H), 2.56-1.92 (m, 8H). MS (ESI, m/e) [M+H]⁺ 664.4.

### Example 76: (3R)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-3-carbonitrile

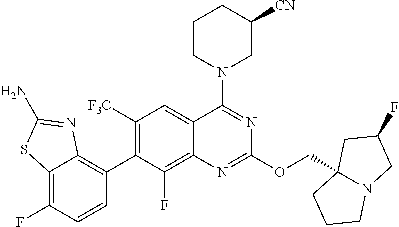 [SMILES: N#C[C@@H]1CCCN(c2nc(OC[C@@]34CCCN3C[C@H](F)C4)nc3c(F)c(-c4ccc(F)c5sc(N)nc45)c(C(F)(F)F)cc23)C1]

Example 76 was prepared by similar procedure as described in Example 1 by replacing cyclopropanamine with (R)-piperidine-3-carbonitrile to give the title product. ¹H NMR (500 MHz, CD₃OD) δ 8.21 (s, 1H), 7.27-7.15 (m, 1H), 7.01-6.91 (m, 1H), 5.52-5.32 (m, 1H), 4.61-4.28 (m, 4H), 4.18-4.10 (m, 1H), 4.05-3.95 (m, 1H), 3.83-3.74 (m, 1H), 3.69-3.50 (m, 3H), 2.56-1.84 (m, 10H). MS (ESI, m/e) [M+H]⁺ 648.4.

### Example 77: (3S)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-3-carbonitrile

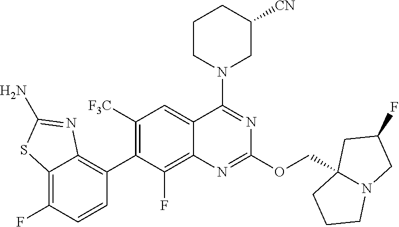 [SMILES: N#C[C@H]1CCCN(c2nc(OC[C@@]34CCCN3C[C@H](F)C4)nc3c(F)c(-c4ccc(F)c5sc(N)nc45)c(C(F)(F)F)cc23)C1]

Example 77 was prepared by similar procedure as described in Example 1 by replacing cyclopropanamine with (3S)-piperidine-3-carbonitrile to give the title product. ¹H NMR (500 MHz, CD₃OD) δ 8.23 (s, 1H), 7.27-7.13 (m, 1H), 7.01-6.91 (m, 1H), 5.63-5.47 (m, 1H), 4.79-4.16 (m, 4H), 4.05-3.78 (m, 5H), 2.69-1.84 (m, 10H). MS (ESI, m/e) [M+H]⁺ 648.4.

### Example 78: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidin-4-ol

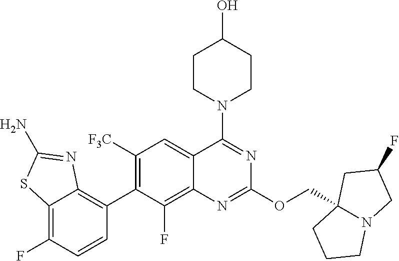 [SMILES: Nc1nc2c(-c3c(C(F)(F)F)cc4c(N5CCC(O)CC5)nc(OC[C@@]56CCCN5C[C@H](F)C6)nc4c3F)ccc(F)c2s1]

Example 78 was prepared by similar procedure as described in Example 2 by replacing (1S,2R)-2-fluorocyclopropan-1-amine with piperidin-4-ol to give the title product. ¹H NMR (500 MHz, CD₃OD) δ 8.14 (s, 1H), 7.21-7.15 (m, 1H), 7.00-6.93 (m, 1H), 5.47-5.29 (m, 1H), 4.47-426 (m, 4H), 4.06-3.97 (m, 1H), 3.75-3.64 (m, 2H), 3.58-3.36 (m, 3H), 3.22-3.12 (m, 1H), 2.49-1.67 (m, 10H). MS (ESI, m/e) [M+H]⁺ 639.4.

### Example 79: (3S)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methylpiperidin-3-ol

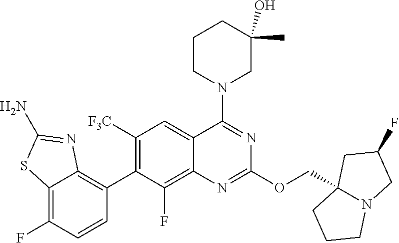 [SMILES: C[C@]1(O)CCCN(c2nc(OC[C@@]34CCCN3C[C@H](F)C4)nc3c(F)c(-c4ccc(F)c5sc(N)nc45)c(C(F)(F)F)cc23)C1]

Example 79 was prepared by similar procedure as described in Example 1 by replacing cyclopropanamine with (S)-3-methylpiperidin-3-ol to give the title product. ¹H NMR (500 MHz, CD₃OD) δ 8.44 (s, 1H), 7.22-7.14 (m, 1H), 7.00-6.92 (m, 1H), 5.48-5.31 (m, 1H), 4.50-4.30 (m, 3H), 4.20-4.12 (m, 1H), 3.56-3.34 (m, 5H), 3.20-3.12 (m, 1H), 2.53-1.73 (m, 10H), 1.27 (s, 3H). MS (ESI, m/e) [M+H]⁺ 653.5.

### Example 80: (3R)-1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methylpiperidin-3-ol

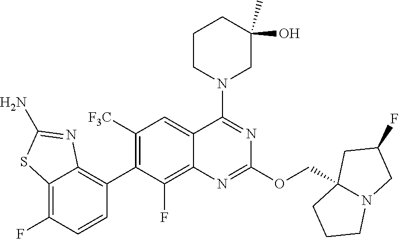 [SMILES: C[C@@]1(O)CCCN(c2nc(OC[C@@]34CCCN3C[C@H](F)C4)nc3c(F)c(-c4ccc(F)c5sc(N)nc45)c(C(F)(F)F)cc23)C1]

Example 80 was prepared by similar procedure as described in Example 1 by replacing cyclopropanamine with (R)-3-methylpiperidin-3-ol to give the title product. ¹H NMR (500 MHz, CD₃OD) δ 8.43 (s, 1H), 7.22-7.14 (m, 1H), 7.00-6.92 (m, 1H), 5.46-5.28 (m, 1H), 4.46-4.30 (m, 3H), 4.19-4.08 (m, 1H), 3.58-3.38 (m, 5H), 3.22-3.14 (m, 1H), 2.47-1.68 (m, 10H), 1.28 (s, 3H). MS (ESI, m/e) [M+H]⁺ 653.5.

### Example 81: 4-(4-(azepan-1-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

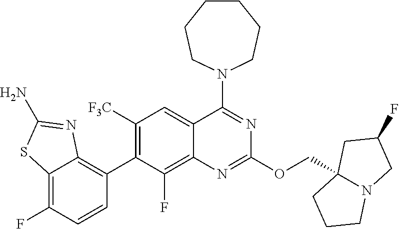 [SMILES: Nc1nc2c(-c3c(C(F)(F)F)cc4c(N5CCCCCC5)nc(OC[C@@]56CCCN5C[C@H](F)C6)nc4c3F)ccc(F)c2s1]

Example 81 was prepared by similar procedure as described in Example 1 by replacing cyclopropanamine with azepane to give the title product. ¹H NMR (500 MHz, CD₃OD) δ 8.32 (s, 1H), 7.23-7.13 (m, 1H), 7.00-6.92 (m, 1H), 5.46-5.26 (m, 1H), 4.45-4.28 (m, 2H), 4.15-3.98 (m, 4H), 3.54-3.32 (m, 3H), 3.17-3.08 (m, 1H), 2.45-1.88 (m, 10H), 1.80-1.62 (m, 4H). MS (ESI, m/e) [M+H]⁺ 637.5.

### Example 82: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carbonitrile

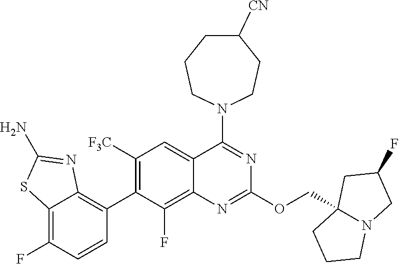 [SMILES: N#CC1CCCN(c2nc(OC[C@@]34CCCN3C[C@H](F)C4)nc3c(F)c(-c4ccc(F)c5sc(N)nc45)c(C(F)(F)F)cc23)CC1]

Example 82 was prepared by similar procedure as described in Example 1 by replacing cyclopropanamine with azepane-4-carbonitrile to give the title product. ¹H NMR (500 MHz, CD₃OD) δ 8.31 (s, 1H), 7.27-7.11 (m, 1H), 7.01-6.92 (m, 1H), 5.46-5.29 (m, 1H), 4.48-4.30 (m, 2H), 4.25-3.96 (m, 4H), 3.60-3.35 (m, 3H), 3.23-3.08 (m, 2H), 2.50-1.87 (m, 12H). MS (ESI, m/e) [M+H]⁺ 662.5.

### Example 83: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-3-carbonitrile

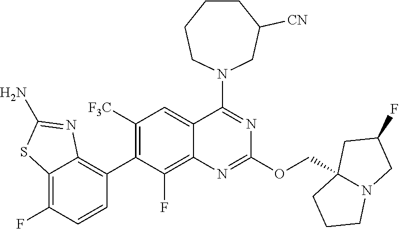 [SMILES: N#CC1CCCCN(c2nc(OC[C@@]34CCCN3C[C@H](F)C4)nc3c(F)c(-c4ccc(F)c5sc(N)nc45)c(C(F)(F)F)cc23)C1]

Example 83 was prepared by similar procedure as described in Example 1 by replacing cyclopropanamine with azepane-3-carbonitrile to give the title product. ¹H NMR (500 MHz, CD₃OD) δ 8.34 (s, 1H), 7.28-7.14 (m, 1H), 7.02-6.92 (m, 1H), 5.50-5.29 (m, 1H), 4.67-4.39 (m, 3H), 4.36-3.98 (m, 3H), 3.70-3.40 (m, 4H), 3.24-3.10 (m, 1H), 2.61-1.78 (m, 11H), 1.65-1.45 (m, 1H). MS (ESI, m/e) [M+H]⁺ 662.4.

### Example 84: 1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepan-4-ol

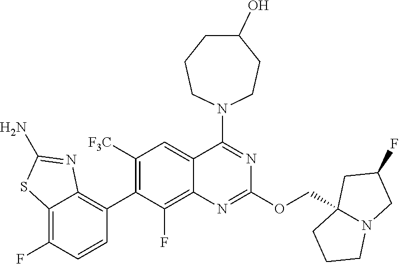 [SMILES: Nc1nc2c(-c3c(C(F)(F)F)cc4c(N5CCCC(O)CC5)nc(OC[C@@]56CCCN5C[C@H](F)C6)nc4c3F)ccc(F)c2s1]

Example 84 was prepared by similar procedure as described in Example 1 by replacing cyclopropanamine with azepan-4-ol to give the title product. ¹H NMR (500 MHz, CD₃OD) δ 8.31 (s, 1H), 7.24-7.13 (m, 1H), 7.01-6.91 (m, 1H), 5.48-5.29 (m, 1H), 4.49-4.28 (m, 2H), 4.21-3.86 (m, 5H), 3.58-3.35 (m, 3H), 3.20-3.08 (m, 1H), 2.48-1.68 (m, 12H). MS (ESI, m/e) [M+H]⁺ 653.5.

### Example 85: (1-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepan-4-yl)methanol

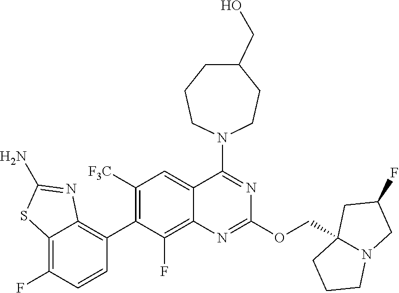 [SMILES: Nc1nc2c(-c3c(C(F)(F)F)cc4c(N5CCCC(CO)CC5)nc(OC[C@@]56CCCN5C[C@H](F)C6)nc4c3F)ccc(F)c2s1]

Example 85 was prepared by similar procedure as described in Example 1 by replacing cyclopropanamine with azepan-4-ylmethanol to give the title product. ¹H NMR (500 MHz, CD₃OD) δ 8.32 (s, 1H), 7.28-7.14 (m, 1H), 7.01-6.91 (m, 1H), 5.49-5.29 (m, 1H), 4.51-4.20 (m, 4H), 4.02-3.86 (m, 2H), 3.64-3.39 (m, 6H), 3.24-3.10 (m, 1H), 2.53-1.62 (m, 12H). MS (ESI, m/e) [M+H]⁺ 667.5.

### Example 86: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-1-oxa-3,8-diazaspiro[4.5]decan-2-one

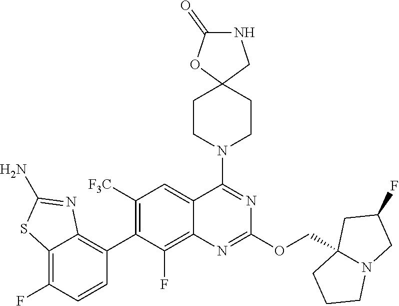 [SMILES: Nc1nc2c(-c3c(C(F)(F)F)cc4c(N5CCC6(CC5)CNC(=O)O6)nc(OC[C@@]56CCCN5C[C@H](F)C6)nc4c3F)ccc(F)c2s1]

Example 86 was prepared by similar procedure as described in Example 2 by replacing (1S,2R)-2-fluorocyclopropan-1-amine with 1-oxa-3,8-diazaspiro[4.5]decan-2-one to give the title product. 1H NMR (500 MHz, CD₃OD) δ 8.17 (s, 1H), 7.24-7.15 (m, 1H), 7.01-6.91 (m, 1H), 5.52-5.32 (m, 1H), 4.55-4.40 (m, 2H), 4.38-4.28 (m, 2H), 3.93-3.79 (m, 2H), 3.71-3.43 (m, 5H), 3.28-3.19 (m, 1H), 2.55-1.95 (m, 10H). MS (ESI, m/e) [M+H]⁺ 694.5.

### Example 87: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-methyl-1-oxa-3,8-diazaspiro[4.5]decan-2-one

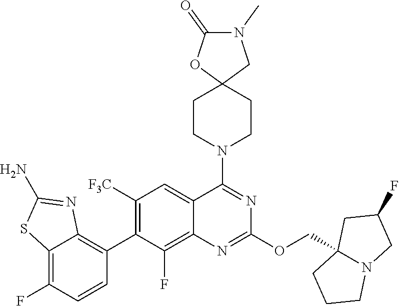 [SMILES: CN1CC2(CCN(c3nc(OC[C@@]45CCCN4C[C@H](F)C5)nc4c(F)c(-c5ccc(F)c6sc(N)nc56)c(C(F)(F)F)cc34)CC2)OC1=O]

Example 87 was prepared by similar procedure as described in Example 1 by replacing cyclopropanamine with 3-methyl-1-oxa-3,8-diazaspiro[4.5]decan-2-one to give the title product. 1H NMR (500 MHz, CD₃OD) δ 8.15 (s, 1H), 7.24-7.14 (m, 1H), 7.01-6.92 (m, 1H), 5.42-5.25 (m, 1H), 4.46-4.26 (m, 4H), 3.86-3.78 (m, 2H), 3.54-3.36 (m, 5H), 3.18-3.04 (m, 1H), 2.86 (s, 3H), 2.43-1.89 (m, 10H). MS (ESI, m/e) [M+H]⁺ 708.6.

### Example 88: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-ethyl-1-oxa-3,8-diazaspiro[4.5]decan-2-one

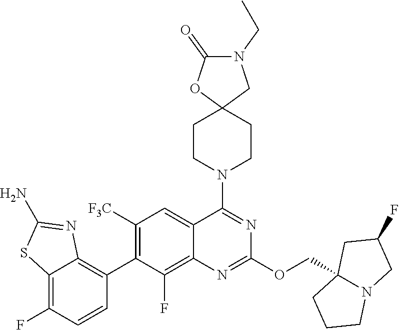 [SMILES: CCN1CC2(CCN(c3nc(OC[C@@]45CCCN4C[C@H](F)C5)nc4c(F)c(-c5ccc(F)c6sc(N)nc56)c(C(F)(F)F)cc34)CC2)OC1=O]

Example 88 was prepared by similar procedure as described in Example 1 by replacing cyclopropanamine with 3-ethyl-1-oxa-3,8-diazaspiro[4.5]decan-2-one to give the title product. ¹H NMR (500 MHz, CD₃OD) δ 8.15 (s, 1H), 7.24-7.14 (m, 1H), 7.01-6.92 (m, 1H), 5.45-5.28 (m, 1H), 4.50-4.31 (m, 4H), 3.96-3.78 (m, 2H), 3.65-3.38 (m, 5H), 3.19-3.10 (m, 1H), 2.52-1.98 (m, 10H), 1.23-1.14 (m, 3H). MS (ESI, m/e) [M+H]⁺ 722.5.

### Example 89: 2-(8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2-oxo-1-oxa-3,8-diazaspiro[4.5]decan-3-yl)acetonitrile

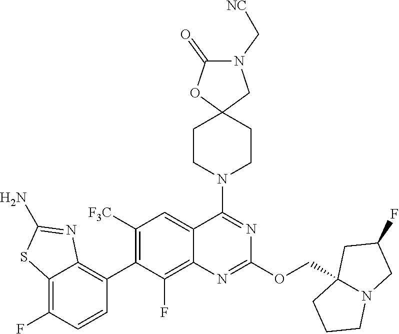 [SMILES: N#CCN1CC2(CCN(c3nc(OC[C@@]45CCCN4C[C@H](F)C5)nc4c(F)c(-c5ccc(F)c6sc(N)nc56)c(C(F)(F)F)cc34)CC2)OC1=O]

Example 89 was prepared by similar procedure as described in Example 1 by replacing cyclopropanamine with 2-(2-oxo-1-oxa-3,8-diazaspiro[4.5]decan-3-yl)acetonitrile to give the title product. ¹H NMR (500 MHz, CD₃OD) δ 8.37 (s, 1H), 7.47-7.30 (m, 1H), 7.27-7.10 (m, 1H), 5.66-5.50 (m, 1H), 4.87-4.67 (m, 3H), 4.39 (s, 2H), 4.15-3.85 (m, 5H), 3.61-3.46 (m, 3H), 2.78-2.09 (m, 10H). MS (ESI, m/e) [M+H]⁺ 733.4.

### Example 90: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-(2,2-difluoroethyl)-1-oxa-3,8-diazaspiro[4.5]decan-2-one

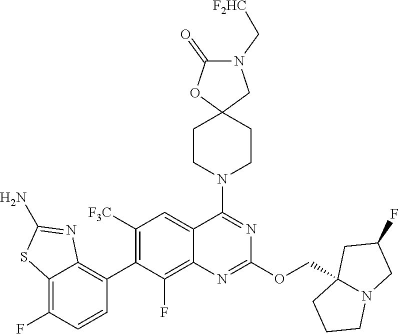 [SMILES: Nc1nc2c(-c3c(C(F)(F)F)cc4c(N5CCC6(CC5)CN(CC(F)F)C(=O)O6)nc(OC[C@@]56CCCN5C[C@H](F)C6)nc4c3F)ccc(F)c2s1]

Example 90 was prepared by similar procedure as described in Example 1 by replacing cyclopropanamine with 3-(2,2-difluoroethyl)-1-oxa-3,8-diazaspiro[4.5]decan-2-one to give the title product. ¹H NMR (500 MHz, CD₃OD) δ 8.17 (s, 1H), 7.23-7.15 (m, 1H), 7.02-6.92 (m, 1H), 6.20-5.90 (m, 1H), 5.52-5.35 (m, 1H), 4.56-4.32 (m, 4H), 3.94-3.78 (m, 2H), 3.74-3.51 (m, 8H), 2.51-2.00 (m, 10H). MS (ESI, m/e) [M+H]⁺ 758.5.

### Example 91: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-(2,2,2-trifluoroethyl)-1-oxa-3,8-diazaspiro[4.5]decan-2-one

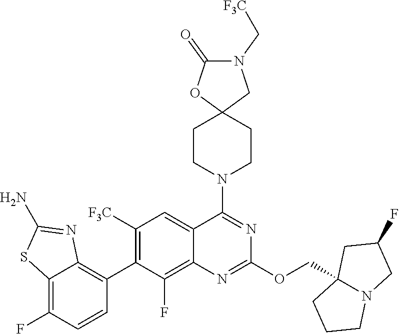 [SMILES: Nc1nc2c(-c3c(C(F)(F)F)cc4c(N5CCC6(CC5)CN(CC(F)(F)F)C(=O)O6)nc(OC[C@@]56CCCN5C[C@H](F)C6)nc4c3F)ccc(F)c2s1]

Example 91 was prepared by similar procedure as described in Example 1 by replacing cyclopropanamine with 3-(2,2,2-trifluoroethyl)-1-oxa-3,8-diazaspiro[4.5]decan-2-one to give the title product. ¹H NMR (500 MHz, CD₃OD) δ 8.15 (s, 1H), 7.24-7.14 (m, 1H), 7.01-6.92 (m, 1H), 5.43-5.26 (m, 1H), 4.42-4.32 (m, 4H), 4.08-3.96 (m, 2H), 3.91-3.82 (m, 2H), 3.63 (s, 2H), 3.20-3.12 (m, 1H), 2.46-1.98 (m, 10H). MS (ESI, m/e) [M+H]⁺ 776.5.

### Example 92: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-(cyclopropylmethyl)-1-oxa-3,8-diazaspiro[4.5]decan-2-one

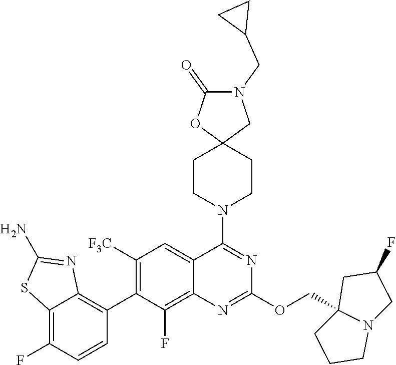 [SMILES: Nc1nc2c(-c3c(C(F)(F)F)cc4c(N5CCC6(CC5)CN(CC5CC5)C(=O)O6)nc(OC[C@@]56CCCN5C[C@H](F)C6)nc4c3F)ccc(F)c2s1]

Example 92 was prepared by similar procedure as described in Example 1 by replacing cyclopropanamine with 3-(cyclopropylmethyl)-1-oxa-3,8-diazaspiro[4.5]decan-2-one to give the title product. ¹H NMR (500 MHz, CD₃OD) δ 8.19 (s, 1H), 7.24-7.15 (m, 1H), 7.02-6.92 (m, 1H), 5.59-5.42 (m, 1H), 4.62-4.50 (m, 2H), 4.43-4.32 (m, 2H), 3.87-3.42 (m, 8H), 3.21-3.14 (m, 2H), 2.65-1.87 (m, 10H), 1.06-0.97 (m, 1H), 0.72-0.53 (m, 2H), 0.34-0.21 (m, 2H). MS (ESI, m/e) [M+H]⁺ 748.4.

### Example 93: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-(2-hydroxyethyl)-1-oxa-3,8-diazaspiro[4.5]decan-2-one

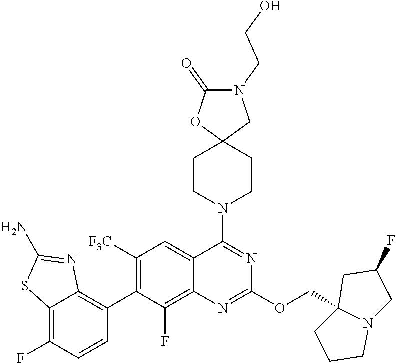 [SMILES: Nc1nc2c(-c3c(C(F)(F)F)cc4c(N5CCC6(CC5)CN(CCO)C(=O)O6)nc(OC[C@@]56CCCN5C[C@H](F)C6)nc4c3F)ccc(F)c2s1]

Example 93 was prepared by similar procedure as described in Example 1 by replacing cyclopropanamine with 3-(2-hydroxyethyl)-1-oxa-3,8-diazaspiro[4.5]decan-2-one to give the title product. ¹H NMR (500 MHz, CD₃OD) δ 8.17 (s, 1H), 7.24-7.15 (m, 1H), 7.02-6.92 (m, 1H), 5.51-5.34 (m, 1H), 4.53-4.29 (m, 4H), 3.92-3.83 (m, 2H), 3.73-3.36 (m, 8H), 3.21-3.10 (m, 2H), 2.51-1.98 (m, 10H). MS (ESI, m/e) [M+H]⁺ 738.5.

### Example 94: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-(2-methoxyethyl)-1-oxa-3,8-diazaspiro[4.5]decan-2-one

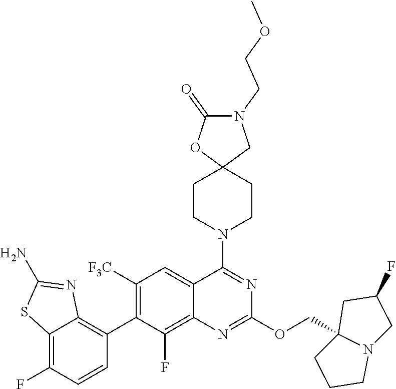 [SMILES: COCCN1CC2(CCN(c3nc(OC[C@@]45CCCN4C[C@H](F)C5)nc4c(F)c(-c5ccc(F)c6sc(N)nc56)c(C(F)(F)F)cc34)CC2)OC1=O]

Example 94 was prepared by similar procedure as described in Example 1 by replacing cyclopropanamine with 3-(2-methoxyethyl)-1-oxa-3,8-diazaspiro[4.5]decan-2-one to give the title product. ¹H NMR (500 MHz, CD₃OD) δ 8.15 (s, 1H), 7.23-7.15 (m, 1H), 7.01-6.92 (m, 1H), 5.45-5.28 (m, 1H), 4.47-4.29 (m, 4H), 3.91-3.79 (m, 2H), 3.65-3.37 (m, 12H), 3.20-3.13 (m, 1H), 2.47-1.90 (m, 10H). MS (ESI, m/e) [M+H]⁺ 752.7.

### Example 95: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2,8-diazaspiro[4.5]decan-1-one

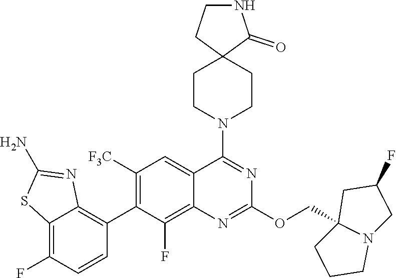 [SMILES: Nc1nc2c(-c3c(C(F)(F)F)cc4c(N5CCC6(CCNC6=O)CC5)nc(OC[C@@]56CCCN5C[C@H](F)C6)nc4c3F)ccc(F)c2s1]

Example 95 was prepared by similar procedure as described in Example 2 by replacing (1S,2R)-2-fluorocyclopropan-1-amine with 2,8-diazaspiro[4.5]decan-1-one to give the title product. ¹HNMR (500 MHz, CD₃OD) δ 8.15 (s, 1H), 7.24-7.15 (m, 1H), 7.01-6.91 (m, 1H), 5.45-5.28 (m, 1H), 4.51-4.32 (m, 4H), 3.74-3.65 (m, 2H), 3.51-3.36 (m, 5H), 3.15-3.08 (m, 1H), 2.48-1.61 (m, 12H). MS (ESI, m/e) [M+H]⁺ 692.5.

### Example 96: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2,8-diazaspiro[4.5]decan-3-one

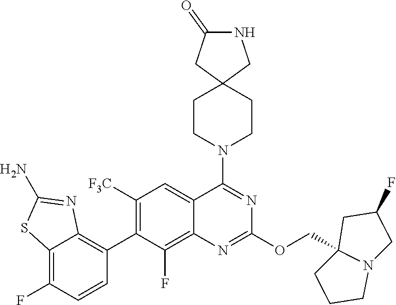 [SMILES: Nc1nc2c(-c3c(C(F)(F)F)cc4c(N5CCC6(CC5)CNC(=O)C6)nc(OC[C@@]56CCCN5C[C@H](F)C6)nc4c3F)ccc(F)c2s1]

Example 96 was prepared by similar procedure as described in Example 2 by replacing (1S,2R)-2-fluorocyclopropan-1-amine with 2,8-diazaspiro[4.5]decan-3-one to give the title product. ¹H NMR (500 MHz, CD₃OD) δ 8.18 (s, 1H), 7.24-7.15 (m, 1H), 7.02-6.92 (m, 1H), 4.65-4.54 (m, 2H), 4.16-4.05 (m, 2H), 4.01-3.79 (m, 5H), 3.48-3.37 (m, 3H), 2.64-1.83 (m, 12H). MS (ESI, m/e) [M+H]⁺ 692.5.

### Example 97: 8-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-2-methyl-2,8-diazaspiro[4.5]decan-3-one

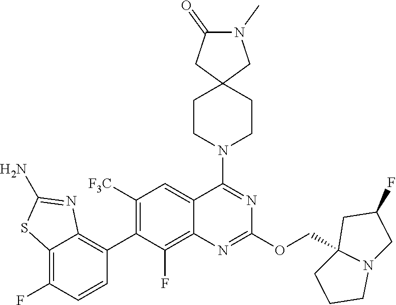 [SMILES: CN1CC2(CCN(c3nc(OC[C@@]45CCCN4C[C@H](F)C5)nc4c(F)c(-c5ccc(F)c6sc(N)nc56)c(C(F)(F)F)cc34)CC2)CC1=O]

Example 97 was prepared by similar procedure as described in Example 2 by replacing (1S,2R)-2-fluorocyclopropan-1-amine with 2-methyl-2,8-diazaspiro[4.5]decan-3-one to give the title product. ¹H NMR (500 MHz, CD₃OD) δ 8.14 (s, 1H), 7.23-7.15 (m, 1H), 7.02-6.92 (m, 1H), 5.47-5.30 (m, 1H), 4.46-4.34 (m, 2H), 4.09-3.95 (m, 4H), 3.53-3.37 (m, 5H), 3.20-3.12 (m, 1H), 2.87 (s, 3H), 2.48-1.78 (m, 12H). MS (ESI, m/e) [M+H]⁺ 706.5.

### Example 98: 9-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-1-oxa-4,9-diazaspiro[5.5]undecan-3-one

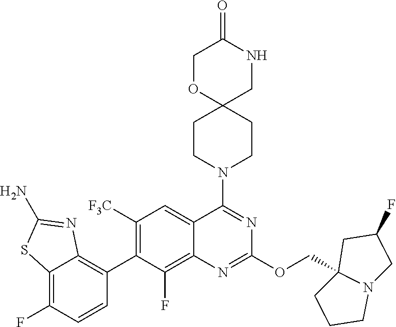 [SMILES: Nc1nc2c(-c3c(C(F)(F)F)cc4c(N5CCC6(CC5)CNC(=O)CO6)nc(OC[C@@]56CCCN5C[C@H](F)C6)nc4c3F)ccc(F)c2s1]

Example 98 was prepared by similar procedure as described in Example 2 by replacing (1S,2R)-2-fluorocyclopropan-1-amine with 1-oxa-4,9-diazaspiro[5.5]undecan-3-one to give the title product. ¹H NMR (500 MHz, CD₃OD) δ 8.18 (s, 1H), 7.23-7.15 (m, 1H), 7.02-6.92 (m, 1H), 5.54-5.37 (m, 1H), 4.56-4.41 (m, 4H), 4.23 (s, 2H), 3.78-3.54 (m, 5H), 2.53-1.86 (m, 10H). MS (ESI, m/e) [M+H]⁺ 708.4.

### Example 99: 9-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-1-oxa-3,9-diazaspiro[5.5]undecan-2-one

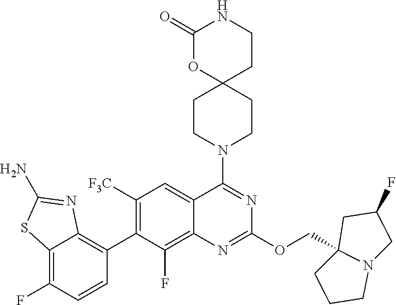 [SMILES: Nc1nc2c(-c3c(C(F)(F)F)cc4c(N5CCC6(CCNC(=O)O6)CC5)nc(OC[C@@]56CCCN5C[C@H](F)C6)nc4c3F)ccc(F)c2s1]

Example 99 was prepared by similar procedure as described in Example 1 by replacing cyclopropanamine with 1-oxa-3,9-diazaspiro[5.5]undecan-2-one to give the title product. ¹H NMR (500 MHz, CD₃OD) δ 8.17 (s, 1H), 7.24-7.15 (m, 1H), 7.01-6.92 (m, 1H), 5.49-5.32 (m, 1H), 4.53-4.38 (m, 4H), 3.95-3.78 (m, 2H), 3.65-3.37 (m, 5H), 2.20-2.12 (m, 1H), 2.53-1.98 (m, 12H). MS (ESI, m/e) [M+H]⁺ 708.5.

### Example 100a (common intermediate): 2-amino-7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[b]thiophene-3-carbonitrile

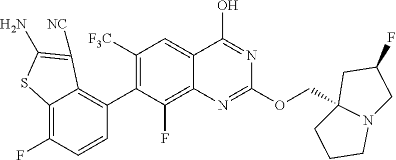 [SMILES: N#Cc1c(N)sc2c(F)ccc(-c3c(C(F)(F)F)cc4c(O)nc(OC[C@@]56CCCN5C[C@H](F)C6)nc4c3F)c12]

### Step 1: tert-butyl (4-(4-(benzyloxy)-8-fluoro-2-(((2R,7aS)-2-fluorotetrahydro-1H-pyrrolizin-7a(5H)-yl) methoxy)-6-(trifluoromethyl) quinazolin-7-yl)-3-cyano-7-fluorobenzo[b]thiophen-2-yl) carbamate

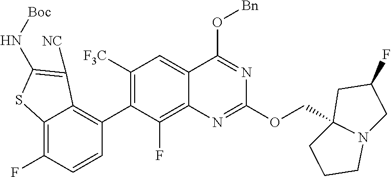 [SMILES: CC(C)(C)OC(=O)Nc1sc2c(F)ccc(-c3c(C(F)(F)F)cc4c(OCc5ccccc5)nc(OC[C@@]56CCCN5C[C@H](F)C6)nc4c3F)c2c1C#N]

To a mixture of 4-(benzyloxy)-7-bromo-8-fluoro-2-(((2R,7aS)-2-fluorotetrahydro-1H-pyrrolizin-7a(5H)-yl) methoxy)-6-(trifluoromethyl) quinazoline (578 mg, 1.04 mmol) in toluene (38 mL) was added tert-butyl (3-cyano-7-fluoro-4-(4,4,5,5-tetramethyl-1,3,2-dioxaborolan-2-yl)benzo[b]thiophen-2-yl)carbamate (1739 mg, 4.160 mmol), DephosPdCl₂ (149 mg, 0.208 mmol) and Cs₂CO₃ (1016 mg, 3.120 mmol) at r.t. The mixture was stirred at 100° C. for 3 hrs. The resulting mixture was concentrated to give a residue which was further purified by flash to give the title product (123 mg).

### Step 2: 2-amino-7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[b]thiophene-3-carbonitrile

To a solution of tert-butyl (4-(4-(benzyloxy)-8-fluoro-2-(((2R,7aS)-2-fluorotetrahydro-1H-pyrrolizin-7a(5H)-yl) methoxy)-6-(trifluoromethyl) quinazolin-7-yl)-3-cyano-7-fluorobenzo[b]thiophen-2-yl) carbamate (123 mg, 0.160 mmol) in DCM (5 mL) was added TFA (5 mL) at r.t, and the mixture was stirred at 45° C. for 2 hrs. The resulting mixture was concentrated to give a residue which was further purified by reversed phase to give the title product (54 mg).

### Example 100: (2R,4S)-1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl) methoxy)-6-(trifluoromethyl) quinazolin-4-yl)-2-methylpiperidine-4-carbonitrile

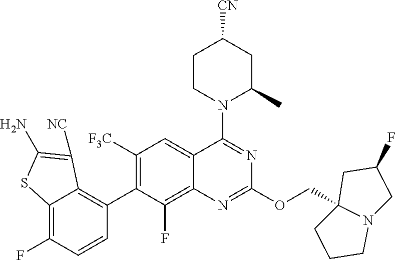 [SMILES: C[C@@H]1C[C@@H](C#N)CCN1c1nc(OC[C@@]23CCCN2C[C@H](F)C3)nc2c(F)c(-c3ccc(F)c4sc(N)c(C#N)c34)c(C(F)(F)F)cc12]

To a mixture of 2-amino-7-fluoro-4-(8-fluoro-2-(((2R,7aS)-2-fluorotetrahydro-1H-pyrrolizin-7a(5H)-yl) methoxy)-4-hydroxy-6-(trifluoromethyl) quinazolin-7-yl) benzo[b]thiophene-3-carbonitrile (50 mg, 0.086 mmol) in acetonitrile (5 mL) was added (2R,4S)-2-methylpiperidine-4-carbonitrile (42 mg, 0.26 mmol), BOP (114 mg, 0.258 mmol) and DIPEA (66 mg, 0.52 mmol). The mixture was stirred for overnight at 40° C. The resulting mixture was concentrated to give a residue which was further purified by Prep-HPLC to give title product (75 mg). ¹H NMR (500 MHz, CD₃OD) δ 8.10-8.00 (m, 1H), 7.26-7.16 (m, 1H), 7.03-6.99 (m, 1H), 5.48-5.30 (m, 1H), 4.99-4.91 (m, 1H), 4.48-4.34 (m, 3H), 3.63-3.33 (m, 5H), 3.20-3.12 (m, 1H), 2.50-1.95 (m, 10H), 1.60-1.49 (m, 3H). MS (ESI, m/e) [M+H]⁺ 686.7.

### Example 101a (common intermediate): 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile

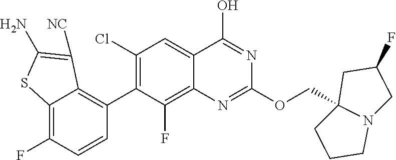 [SMILES: N#Cc1c(N)sc2c(F)ccc(-c3c(Cl)cc4c(O)nc(OC[C@@]56CCCN5C[C@H](F)C6)nc4c3F)c12]

### Step 1: tert-butyl (4-(4-(benzyloxy)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate

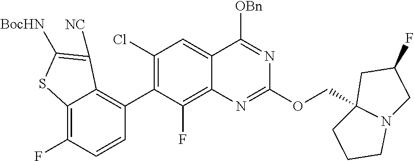 [SMILES: CC(C)(C)OC(=O)Nc1sc2c(F)ccc(-c3c(Cl)cc4c(OCc5ccccc5)nc(OC[C@@]56CCCN5C[C@H](F)C6)nc4c3F)c2c1C#N]

To a 100 mL round-bottomed flask was added 4-(benzyloxy)-7-bromo-6-chloro-8-fluoro-2-(((2R,7aS)-2-fluorotetrahydro-1H-pyrrolizin-7a(5H)-yl)methoxy)quinazoline (600 mg, 1.14 mmol), tert-butyl (3-cyano-7-fluoro-4-(4,4,5,5-tetramethyl-1,3,2-dioxaborolan-2-yl)benzo[b]thiophen-2-yl)carbamate (1.43 g, 3.42 mmol), Cs₂CO₃ (1.11 g, 3.42 mmol), PdCl₂(DPEphos) (122 mg, 0.17 mmol) and toluene (40 mL), and the reaction mixture was stirred at 100° C. for 4 hrs. The resulting cooled mixture was concentrated and purified by flash column chromatography (DCM/MeOH=20/1) to give the title product (295 mg, 35%). MS (ESI, m/e) [M+H]⁺ 736.3.

### Step 2: 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile

To a 50 mL round-bottomed flask was added tert-butyl (4-(4-(benzyloxy)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (295 mg, 0.40 mmol) and TFA (20 mL), and the resulting mixture was stirred at 40° C. for 6 hrs. The reaction mixture was concentrated under reduced pressure. The residue was purified by reverse flash (eluting with H₂O (0.2% FA)/CH₃CN) to give the title product (82 mg, 37%). MS (ESI, m/e) [M+H]⁺ 546.1.

### Example 101: (2R,4S)-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-2-methylpiperidine-4-carbonitrile

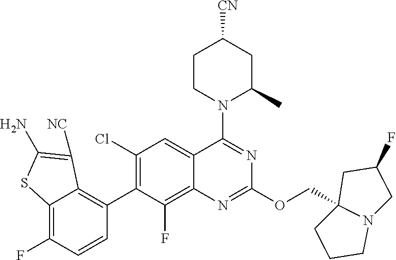 [SMILES: C[C@@H]1C[C@@H](C#N)CCN1c1nc(OC[C@@]23CCCN2C[C@H](F)C3)nc2c(F)c(-c3ccc(F)c4sc(N)c(C#N)c34)c(Cl)cc12]

To a 50 mL round-bottomed flask was added 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (82 mg, 0.15 mmol), (2R,4S)-2-methylpiperidine-4-carbonitrile hydrochloride, BOP (99 mg, 0.22 mmol), DIEA (78 mg, 0.60 mmol) and CH₃CN (8.0 mL), and the resulting mixture was stirred at 40° C. for 24 hrs. The reaction mixture was concentrated under reduced pressure. The residue was purified by reverse flash (eluting with H₂O (0.2% FA)/CH₃CN) to give the title product (45.1 mg, 46%). ¹H NMR (500 MHz, CD₃OD) δ 7.76 (s, 1H), 7.24-7.14 (m, 1H), 7.10-6.96 (m, 1H), 5.40-5.19 (m, 1H), 4.37-4.13 (m, 3H), 3.60-3.48 (m, 1H), 3.27-3.10 (m, 4H), 3.06-2.94 (m, 1H), 2.39-1.80 (m, 10H), 1.54-1.43 (m, 3H). MS (ESI, m/e) [M+H]⁺ 652.4.

### Example 102: (3S)-1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)pyrrolidin-3-ol

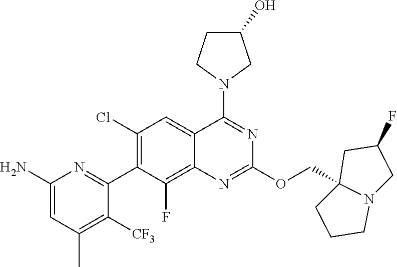 [SMILES: Cc1cc(N)nc(-c2c(Cl)cc3c(N4CC[C@H](O)C4)nc(OC[C@@]45CCCN4C[C@H](F)C5)nc3c2F)c1C(F)(F)F]

### Step 1: benzyl (S)-3-acetoxypyrrolidine-1-carboxylate

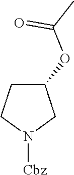 [SMILES: CC(=O)O[C@H]1CCN(C(=O)OCc2ccccc2)C1]

To a solution of benzyl (S)-3-hydroxypyrrolidine-1-carboxylate (2.21 g, 10 mmol) in DCM (40 mL), acetyl chloride (1.02 g, 13 mmol) and DIPEA (3.58 g, 20 mmol) was added. The mixture was stirred at room temperature overnight. The mixture was concentrated in vacuo, and the residue was purified by silica gel column chromatography (DCM/MeOH=7/3) to afford the title compound (2.34 g, 88%). MS (ESI, m/e) [M+H]⁺ 264.1.

### Step 2: (S)-pyrrolidin-3-yl acetate

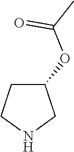

To a solution of benzyl (S)-3-acetoxypyrrolidine-1-carboxylate (2.21 g, 10 mmol) in THF (80 mL), Pd/C (10%, 468 mg) was added in one portion. The mixture was stirred at room temperature overnight. Solid was filtered off, and the filtrate was concentrated in vacuo to afford the title compound (1.18 g, crude), which was used in the next step without further purification. MS (ESI, m/e) [M+H]⁺ 130.2.

### Step 3: (S)-1-(7-bromo-2,6-dichloro-8-fluoroquinazolin-4-yl)pyrrolidin-3-yl acetate

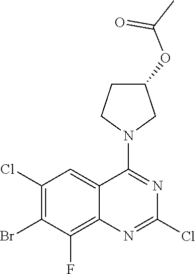 [SMILES: CC(=O)O[C@H]1CCN(c2nc(Cl)nc3c(F)c(Br)c(Cl)cc23)C1]

To a solution of 7-bromo-2,4,6-trichloro-8-fluoroquinazoline (2.5 g, 7.62 mmol) in DCM (30 mL), (S)-pyrrolidin-3-yl acetate (1.18 g, 9.15 mmol) and DIPEA (2 g, 15.2 mmol) was added in one portion. The mixture was stirred at room temperature overnight. The mixture was concentrated in vacuo, the residue was purified by silica gel column chromatography (DCM/MeOH=4/1) to afford the title compound (2.96 g, 92%). MS (ESI, m/e) [M+H]⁺ 422.0.

### Step 4: (S)-1-(7-bromo-6-chloro-2,8-difluoroquinazolin-4-yl)pyrrolidin-3-yl acetate

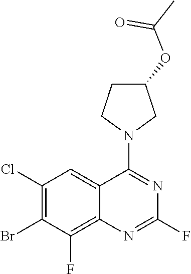 [SMILES: CC(=O)O[C@H]1CCN(c2nc(F)nc3c(F)c(Br)c(Cl)cc23)C1]

A mixture of 7-bromo-2,4,6-trichloro-8-fluoroquinazoline (2.96 g, 7.03 mmol) and KF (4.1 g, 70.3 mmol) in DMSO (30 mL) was stirred at 110° C. for 2 hrs. The mixture was cooled to room temperature, and poured into ice water (300 mL). Precipitate was filtered from the suspension and dried in vacuo to give the title compound (2.29 g, 80%). MS (ESI, m/e) [M+H]⁺ 406.1.

### Step 5: (S)-1-(7-(6-(bis(4-methoxybenzyl)amino)-4-methylpyridin-2-yl)-6-chloro-2,8-difluoroquinazolin-4-yl)pyrrolidin-3-yl acetate

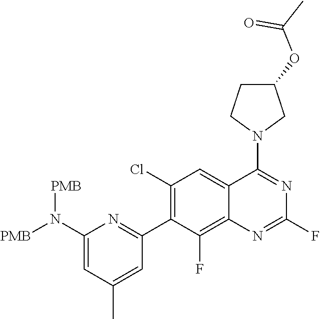

A mixture of (S)-1-(7-bromo-6-chloro-2,8-difluoroquinazolin-4-yl)pyrrolidin-3-yl acetate (581 mg, 1.43 mmol), (6-(bis(4-methoxybenzyl)amino)-4-methylpyridin-2-yl)boronic acid (900 mg, 2.3 mmol), Pd(dppf)Cl₂ (104.5 mg, 0.143 mmol) and K₃PO₄ (756 mg, 3.57 mmol) in THF/water (15 mL/3 mL) was stirred at 65° C. for 2 hrs. The reaction mixture was cooled to room temperature, and concentrated in vacuo. The residue was purified by silica gel column chromatography (PE/EA=1/4) to give the title product (274 mg, 28%). MS (ESI, m/e) [M+H]⁺ 674.5.

### Step 6: (3S)-1-(7-(6-(bis(4-methoxybenzyl)amino)-3-iodo-4-methylpyridin-2-yl)-6-chloro-2,8-difluoroquinazolin-4-yl)pyrrolidin-3-yl acetate

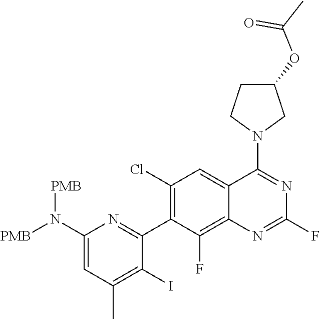

To a stirred solution of (S)-1-(7-(6-(bis(4-methoxybenzyl)amino)-4-methylpyridin-2-yl)-6-chloro-2,8-difluoroquinazolin-4-yl)pyrrolidin-3-yl acetate (274 mg, 0.4 mmol) in DMF/AcOH (6 mL/3 mL) was added NIS (274 mg, 1.22 mmol) in dropwise at 0° C. the mixture was stirred at room temperature for 1.5 hrs. The mixture was extracted with EtOAc (30 mL*3). Combined organic phase was washed with brine (40 mL), NaHCO₃ solution (40 mL) and dried over Na₂SO₄. The mixture was concentrated in vacuo, and the residue was purified by silica gel column chromatography (DCM/MeOH=8/2) to give the title product (116 mg, 35%). MS (ESI, m/e) [M+H]⁺ 800.4.

### Step 7: (3S)-1-(7-(6-(bis(4-methoxybenzyl)amino)-4-methyl-3-(trifluoromethyl)pyridin-2-yl)-6-chloro-2,8-difluoroquinazolin-4-yl)pyrrolidin-3-yl acetate

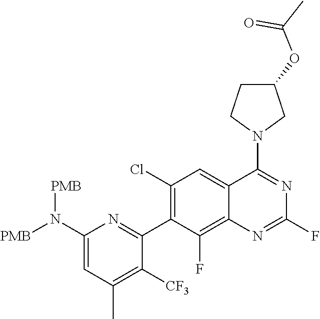

A mixture of (3S)-1-(7-(6-(bis(4-methoxybenzyl)amino)-3-iodo-4-methylpyridin-2-yl)-6-chloro-2,8-difluoroquinazolin-4-yl)pyrrolidin-3-yl acetate (116 mg, 0.145 mmol), CuI (275 mg, 1.45 mmol) and methyl 2,2-difluoro-2-(fluorosulfonyl)acetate (139 mg, 0.725 mmol) in DMA (10 ml) was stirred at 120° C. for 2 hrs. The reaction was cooled to room temperature, diluted with ice-cold water, and then extracted with EtOAc (30 mL*3). Combined organic phase was washed with brine (40 mL) and dried over Na₂SO₄. The residue was purified by silica gel column chromatography (PE/EA=1/4) to give the title product (80 mg, 48%). MS (ESI, m/e) [M+H]⁺ 742.5.

### Step 8: (3S)-1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)pyrrolidin-3-ol

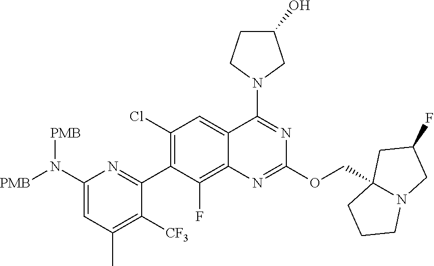

To a solution of ((2R,7aS)-2-fluorotetrahydro-1H-pyrrolizin-7a(5H)-yl)methanol (35 mg, 0.221 mmol) in THF (10 mL) was added NaH (16 mg, 0.333 mmol) at 0° C., and the mixture was stirred at 0° C. for 30 mins. Then, (3S)-1-(7-(6-(bis(4-methoxybenzyl)amino)-3-iodo-4-methylpyridin-2-yl)-6-chloro-2,8-difluoroquinazolin-4-yl)pyrrolidin-3-yl acetate (82 mg, 0.111 mmol) was added. The mixture was stirred from 0° C. to room temperature, and then stirred for another 2 hrs at room temperature. The mixture was quenched by water (1 mL), extracted with EtOAc (20 mL*2). The combined organic phase was washed with brine (20 mL), dried over Na₂SO₄, and concentrated in vacuo. The mixture was purified by silica gel column chromatography (DCM/MeOH=4/1) to give the title product (108 mg, crude). MS (ESI, m/e) [M+H]⁺ 742.5.

### Step 9: (3S)-1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)pyrrolidin-3-ol

A mixture of (3S)-1-(7-(6-(bis(4-methoxybenzyl)amino)-4-methyl-3-(trifluoromethyl)pyridin-2-yl)-6-chloro-2,8-difluoroquinazolin-4-yl)pyrrolidin-3-yl acetate (108 mg, 0.129 mmol) and TFA (5 mL). The mixture was stirred at 50° C. for 1 h. The solvent was removed in vacuo to give the crude product which was purified by prep-HPLC to give the title product (1.28 mg, 1.6%). ¹H NMR (500 MHz, DMSO-d₆) δ 8.09 (s, 1H), 6.86 (s, 2H), 6.49 (s, 1H), 5.49-5.28 (m, 1H), 5.09 (s, 1H), 4.43 (s, 1H), 4.35-3.67 (m, 6H), 3.28-2.70 (m, 4H), 2.37 (s, 3H), 2.27-1.64 (m, 8H). MS (ESI, m/e) [M+H]⁺ 599.4.

### Example 103: 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

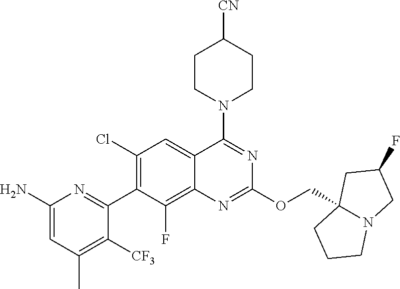 [SMILES: Cc1cc(N)nc(-c2c(Cl)cc3c(N4CCC(C#N)CC4)nc(OC[C@@]45CCCN4C[C@H](F)C5)nc3c2F)c1C(F)(F)F]

### Step 1: 1-(7-bromo-2,6-dichloro-8-fluoroquinazolin-4-yl)piperidine-4-carbonitrile

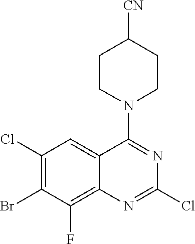 [SMILES: N#CC1CCN(c2nc(Cl)nc3c(F)c(Br)c(Cl)cc23)CC1]

To a solution of 7-bromo-2,4,6-trichloro-8-fluoroquinazoline (2.5 g, 7.62 mmol) in DCM (100 mL), piperidine-4-carbonitrile (0.924 g, 8.38 mmol) and DIPEA (3.24 g, 25.1 mmol) was added. The mixture was stirred at room temperature overnight. The mixture was concentrated in vacuo, and the residue was purified by silica gel column chromatography (PE/EA=7/3) to afford the title compound (390 mg, 13%). MS (ESI, m/e) [M+H]⁺ 403.1.

### Step 2: 1-(7-bromo-6-chloro-2,8-difluoroquinazolin-4-yl)piperidine-4-carbonitrile

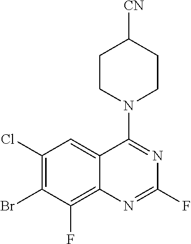 [SMILES: N#CC1CCN(c2nc(F)nc3c(F)c(Br)c(Cl)cc23)CC1]

A mixture of 1-(7-bromo-2,6-dichloro-8-fluoroquinazolin-4-yl)piperidine-4-carbonitrile (385 mg, 0.958 mmol) and KF (278 mg, 4.79 mmol) in DMSO (10 mL) was stirred at 115° C. for 3 hours. The mixture was cooled to room temperature, and poured into ice water (150 mL). Solid was filtered as crude product, which was purified by silica gel column chromatography (PE/EA=19/1) to afford the title compound (269 mg, 72%). MS (ESI, m/e) [M+H]⁺ 387.1.

### Step 3: 1-(7-(6-(bis(4-methoxybenzyl)amino)-4-methylpyridin-2-yl)-6-chloro-2,8-difluoroquinazolin-4-yl)piperidine-4-carbonitrile

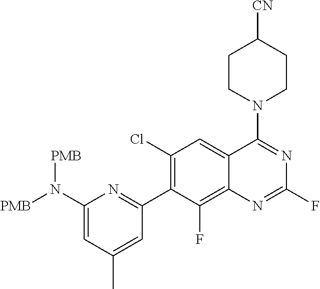

A mixture of 1-(7-bromo-6-chloro-2,8-difluoroquinazolin-4-yl)piperidine-4-carbonitrile (982 mg, 2.54 mmol), (6-(bis(4-methoxybenzyl)amino)-4-methylpyridin-2-yl)boronic acid (1.75 g, 4.45 mmol), Pd(dppf)Cl₂ (185 mg, 0.25 mmol) and K₃PO₄ (1.08 g, 5.08 mmol) in THF/water (30 mL/6 mL) was stirred at 65° C. for 2 hours. The reaction mixture was cooled to room temperature, and concentrated in vacuo. The residue was purified by silica gel column chromatography (PE:EA=100%:0%-0%:100%) to give the title product (670 mg, 40%). MS (ESI, m/e) [M+H]⁺ 655.5.

### Step 4: 1-(7-(6-(bis(4-methoxybenzyl)amino)-3-iodo-4-methylpyridin-2-yl)-6-chloro-2,8-difluoroquinazolin-4-yl)piperidine-4-carbonitrile

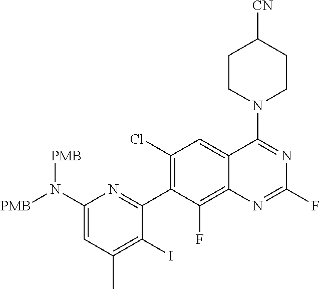

To the solution of 1-(7-(6-(bis(4-methoxybenzyl)amino)-4-methylpyridin-2-yl)-6-chloro-2,8-difluoroquinazolin-4-yl)piperidine-4-carbonitrile (570 mg, 0.871 mmol) in DMF/AcOH (20 mL/10 mL) was added NIS (784 mg, 3.486 mmol). The mixture was stirred at room temperature for 1.5 hrs. The mixture was extracted with EtOAc (50 mL*3). Combined organic phase was washed with brine (80 mL), Na₂S₂O₃ solution (40 mL), and dried over Na₂SO₄. The mixture was concentrated in vacuo, and the residue was purified by silica gel column chromatography (DCM/MeOH=10/1) to give the title product (550 mg, 81%). MS (ESI, m/e) [M+H]⁺ 781.5.

### Step 5: 1-(7-(6-(bis(4-methoxybenzyl)amino)-4-methyl-3-(trifluoromethyl)pyridin-2-yl)-6-chloro-2,8-difluoroquinazolin-4-yl)piperidine-4-carbonitrile

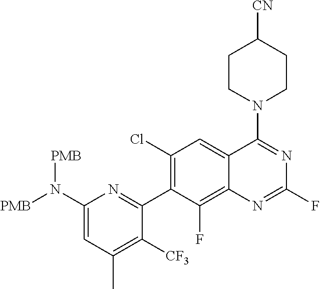

A mixture of 1-(7-(6-(bis(4-methoxybenzyl)amino)-3-iodo-4-methylpyridin-2-yl)-6-chloro-2,8-difluoroquinazolin-4-yl)piperidine-4-carbonitrile (550 mg, 1.964 mmol), CuI (2.02 g, 9.82 mmol) and methyl 2,2-difluoro-2-(fluorosulfonyl)acetate (746 mg, 3.93 mmol) in DMA (20 mL) was stirred at 120° C. for 2 hrs. Cooled reaction mixture was extracted with EtOAc (50 mL*3). Combined organic phase was washed with brine (50 mL), dried over Na₂SO₄ and concentrated in vacuo. The residue was purified by silica gel column chromatography (PE/EA=1/1) to give the title product (455 mg, 89%). MS (ESI, m/e) [M+H]⁺ 723.5.

### Step 6: 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

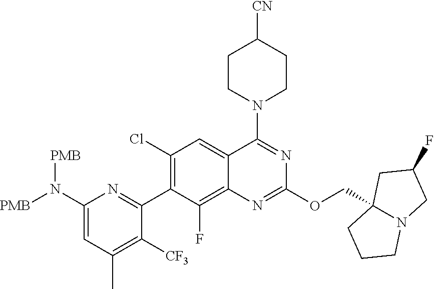

To a solution of ((2R,7aS)-2-fluorotetrahydro-1H-pyrrolizin-7a(5H)-yl)methanol (150 mg, 0.943 mmol) in THF (10 mL) was added NaH (50 mg, 1.25 mmol) at 0° C. The mixture was stirred at 0° C. for 30 mins. Then, 1-(7-(6-(bis(4-methoxybenzyl)amino)-4-methyl-3-(trifluoromethyl)pyridin-2-yl)-6-chloro-2,8-difluoroquinazolin-4-yl)piperidine-4-carbonitrile (455 mg, 0.625 mmol) was added. The mixture was stirred from 0° C. to room temperature, and then stirred for another 3 hrs at room temperature. The mixture was quenched with water (1 mL) and concentrated in vacuo. The residue was purified by silica gel column chromatography (PE/EA=1/4) to give the title product (390 mg, 72%). MS (ESI, m/e) [M+H]⁺ 862.7.

### Step 7: 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

A mixture of 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile (390 mg, 0.452 mmol) and TFA (5 mL). The mixture was stirred at 50° C. for 2.5 hrs. The solvent was removed in vacuo to give the crude product, which was purified by prep-HPLC to give the title product (245 mg, 87%). ¹H NMR (500 MHz, DMSO-d₆) δ 7.82 (s, 1H), 6.86 (s, 2H), 6.50 (s, 1H), 5.50-5.22 (m, 1H), 4.40-3.90 (m, 4H), 3.66-3.55 (m, 2H), 3.28-3.10 (m, 3H), 3.02-2.90 (m, 1H), 2.37 (s, 3H), 2.25-1.73 (m, 10H). MS (ESI, m/e) [M+H]⁺ 622.5.

### Example 104: 1-(7-(6-amino-4-methyl-3-(trifluoromethyl) pyridin-2-yl)-6-chloro-8-fluoro-2-(((2R,7aS)-2-fluorotetrahydro-1H-pyrrolizin-7a(5H)-yl) methoxy) quinazolin-4-yl)-trans-2-methylpiperidine-4-carbonitrile

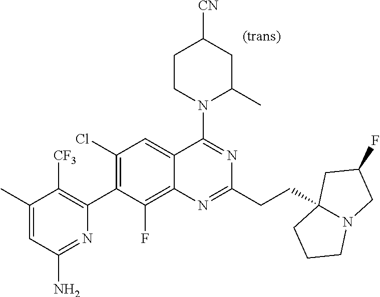 [SMILES: Cc1cc(N)nc(-c2c(Cl)cc3c(N4CCC(C#N)CC4C)nc(CC[C@@]45CCCN4C[C@H](F)C5)nc3c2F)c1C(F)(F)F]

### Step 1: 1-(7-bromo-2,6-dichloro-8-fluoroquinazolin-4-yl)-trans-2-methylpiperidine-4-carbonitrile

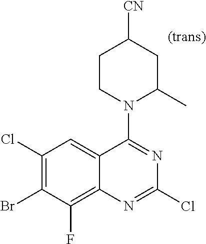 [SMILES: CC1CC(C#N)CCN1c1nc(Cl)nc2c(F)c(Br)c(Cl)cc12]

To a solution of 7-bromo-2,4,6-trichloro-8-fluoroquinazoline (759 mg, 2.3 mmol) in DCM (25 mL) was added trans-2-methylpiperidine-4-carbonitrile (370 mg, 2.3 mmol) and DIPEA (890 mg, 6.9 mmol) at room temperature, and the mixture was stirred at room temperature for 1 h. The resulting mixture was concentrated and the residue was purified by chromatography column on silica (eluting with PE/EA=5/1) to give the title product (700 mg, 73%). MS (ESI, m/e) [M+H]⁺ 417.0.

### Step 2: 1-(7-bromo-6-chloro-2,8-difluoroquinazolin-4-yl)-trans-2-methylpiperidine-4-carbonitrile

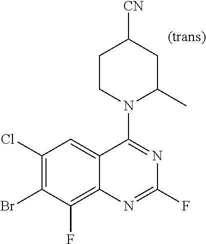 [SMILES: CC1CC(C#N)CCN1c1nc(F)nc2c(F)c(Br)c(Cl)cc12]

To a solution of 1-(7-bromo-2,6-dichloro-8-fluoroquinazolin-4-yl)-trans-2-methylpiperidine-4-carbonitrile (700 mg, 1.68 mmol) in DMSO (30 mL) was added KF (292 mg, 5.04 mmol) at room temperature, and the mixture was stirred at 100° C. for overnight. The resulting mixture was diluted with water (20 mL) and extracted with DCM (30 mL*3), and the combined organic layer was concentrated to give a residue which was purified by chromatography column on silica (eluting with PE/EA=10/1) to give the title product (370 mg, 53%). MS (ESI, m/e) [M+H]⁺ 401.0.

### Step 3: 1-(7-(6-(bis(4-methoxybenzyl) amino)-4-methylpyridin-2-yl)-6-chloro-2,8-difluoroquinazolin-4-yl)-trans-2-methylpiperidine-4-carbonitrile

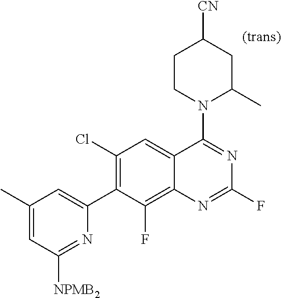

To a solution of 1-(7-bromo-6-chloro-2,8-difluoroquinazolin-4-yl)-trans-2-methylpiperidine-4-carbonitrile (304 mg, 0.758 mmol) in dioxane (30 mL) was added (6-(bis(4-methoxybenzyl) amino)-4-methylpyridin-2-yl)boronic acid (743 mg, 1.895 mmol), Pd(dppf)Cl₂ (124 mg, 0.15 mmol), K₃PO₄ (482 mg, 2.274 mmol) and water (6 mL) at room temperature, and the mixture was stirred at 100° C. for 2 hrs. The resulting mixture was concentrated and the crude product was purified by chromatography column on silica (eluting with DCM/MeOH=20/1) to give the title product (344 mg, 38%). MS (ESI, m/e) [M+H]⁺ 669.0.

### Step 4: 1-(7-(6-(bis(4-methoxybenzyl) amino)-3-iodo-4-methylpyridin-2-yl)-6-chloro-2,8-difluoroquinazolin-4-yl)-trans-2-methylpiperidine-4-carbonitrile

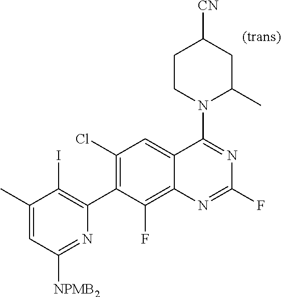

To a solution of 1-(7-(6-(bis(4-methoxybenzyl) amino)-4-methylpyridin-2-yl)-6-chloro-2,8-difluoroquinazolin-4-yl)-trans-2-methylpiperidine-4-carbonitrile (324 mg, 0.485 mmol) in DMF (10 mL) was added I₂ (859 mg, 3.395 mmol) and Ag₂SO₄ (45 mg, 0.1455 mmol) at room temperature, and the mixture was stirred at room temperature for 2 hrs. The resulting mixture was diluted with water (20 mL) and extracted with DCM (30 mL*3), and the combined organic layer was concentrated to give crude product (304 mg). MS (ESI, m/e) [M+H]⁺ 795.0.

### Step 5: 1-(7-(6-(bis(4-methoxybenzyl) amino)-4-methyl-3-(trifluoromethyl) pyridin-2-yl)-6-chloro-2,8-difluoroquinazolin-4-yl)-trans-2-methylpiperidine-4-carbonitrile

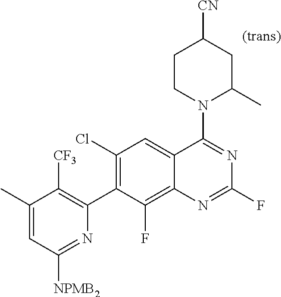

To a solution of 1-(7-(6-(bis(4-methoxybenzyl) amino)-3-iodo-4-methylpyridin-2-yl)-6-chloro-2,8-difluoroquinazolin-4-yl)-trans-2-methylpiperidine-4-carbonitrile (304 mg, 0.382 mmol) in DMF (10 mL) was added methyl 2,2-difluoro-2-(fluorosulfonyl)acetate (735 mg, 3.83 mmol) and CuI (728 mg, 3.83 mmol) at room temperature, and the mixture was stirred at 90° C. for 4 hrs. The resulting mixture was concentrated and the crude product was purified by chromatography column on silica (eluting with DCM/MeOH=20/1) to give the title product (107 mg, 38%). MS (ESI, m/e) [M+H]⁺ 737.0.

### Step 6: 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-trans-2-methylpiperidine-4-carbonitrile

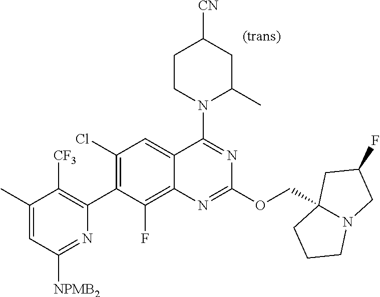

To a solution of 1-(7-(6-(bis(4-methoxybenzyl) amino)-4-methyl-3-(trifluoromethyl) pyridin-2-yl)-6-chloro-2,8-difluoroquinazolin-4-yl)-trans-2-methylpiperidine-4-carbonitrile (50 mg, 0.068 mmol) in THF (5 mL) was added ((2R,7aS)-2-fluorotetrahydro-1H-pyrrolizin-7a(5H)-yl)methanol (27 mg, 0.17 mmol) and NaH (7 mg, 0.17 mmol) at room temperature, and the mixture was stirred at room temperature for 1 h. The resulting mixture was quenched with ice water and extracted with DCM (30 mL*3), and the combined organic layer was concentrated to give crude product (50 mg). MS (ESI, m/e) [M+H]⁺ 842.0.

### Step 6: 1-(7-(6-amino-4-methyl-3-(trifluoromethyl) pyridin-2-yl)-6-chloro-8-fluoro-2-(((2R,7aS)-2-fluorotetrahydro-1H-pyrrolizin-7a(5H)-yl) methoxy) quinazolin-4-yl)-trans-2-methylpiperidine-4-carbonitrile

To a solution of 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-trans-2-methylpiperidine-4-carbonitrile (50 mg, 0.057 mmol) in DCM (1 mL) was added TFA (4 mL) at room temperature. The mixture was stirred at 50° C. for 1 h. The resulting mixture was concentrated at room temperature and pH was adjusted to 7 with Na₂CO₃, and the organic layer was concentrated to give a residue which was further purified by Prep-HPLC to give the title product (16.56 mg). ¹H NMR (500 MHz, CD₃OD) δ 7.76 (s, 1H), 6.60 (s, 1H), 5.50-5.32 (m, 1H), 4.97-4.88 (m, 1H), 4.50-4.35 (m, 2H), 4.34-4.24 (m, 1H), 3.65-3.43 (m, 4H), 3.39-3.32 (m, 1H), 3.24-3.15 (m, 1H), 2.55-1.95 (m, 13H), 1.58-1.39 (m, 3H). MS (ESI, m/e) [M+H]⁺ 636.6.

### Example 105: 6-(6-chloro-8-fluoro-2-(((2R,7aS)-2-fluorotetrahydro-1H-pyrrolizin-7a(5H)-yl)methoxy)-4-((S)-3-methylmorpholino)quinazolin-7-yl)-4-methyl-5-(trifluoromethyl)pyridin-2-amine

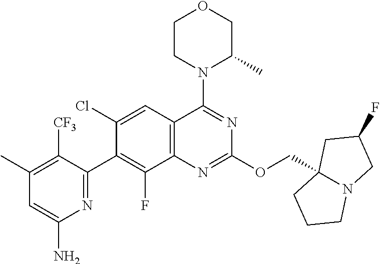 [SMILES: Cc1cc(N)nc(-c2c(Cl)cc3c(N4CCOC[C@@H]4C)nc(OC[C@@]45CCCN4C[C@H](F)C5)nc3c2F)c1C(F)(F)F]

### Step 1: (S)-4-(7-bromo-2,6-dichloro-8-fluoroquinazolin-4-yl)-3-methylmorpholine

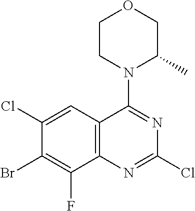 [SMILES: C[C@H]1COCCN1c1nc(Cl)nc2c(F)c(Br)c(Cl)cc12]

To a solution of 7-bromo-2,4,6-trichloro-8-fluoroquinazoline (1.60 g, 4.85 mmol) in dichloromethane (15 mL) was added (S)-3-methylmorpholine (600 mg, 5.93 mmol), and 2 mL N,N-diisopropylethylamine at 0° C. The reaction was stirred at room temperature for 0.5 h. The mixture was diluted with dichloromethane and water. Combined organic layer was dried over sodium sulfate and evaporated. The residue was purified by chromatography on silica to give the title compound (1.20 g, 63%). MS (ESI, m/e) [M+H]⁺ 393.5.

### Step 2: (S)-4-(7-bromo-6-chloro-2,8-difluoroquinazolin-4-yl)-3-methylmorpholine

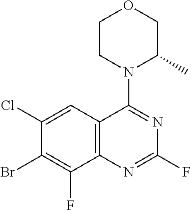 [SMILES: C[C@H]1COCCN1c1nc(F)nc2c(F)c(Br)c(Cl)cc12]

To a solution of (S)-4-(7-bromo-2,6-dichloro-8-fluoroquinazolin-4-yl)-3-methyl morpholine (1.20 g, 3.03 mmol) in 30 mL dimethyl sulfoxide was added potassium fluoride (1.21 g, 20.86 mmol). The reaction was stirred at 95° C. for 16 hrs. The mixture was cooled to room temperature, and diluted with dichloromethane and water. Combined organic layer was dried over sodium sulfate and evaporated. The residue was purified by chromatography on silica to give the title compound (950 mg, 82%). MS (ESI, m/e) [M+H]⁺ 377.5.

### Step 3: (S)-6-(6-chloro-2,8-difluoro-4-(3-methylmorpholino)quinazolin-7-yl)-N,N-bis(4-methoxybenzyl)-4-methylpyridin-2-amine

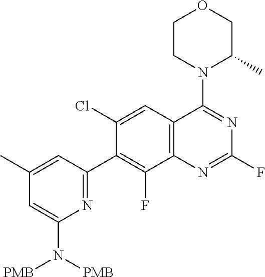

To a solution of (S)-4-(7-bromo-6-chloro-2,8-difluoroquinazolin-4-yl)-3-methyl morpholine (368 mg, 0.98 mmol) in 10 mL 1,4-dioxane was added N,N-bis(4-methoxybenzyl)-4-methyl-6-(tributylstannyl)pyridin-2-amine (1.30 g, 2.04 mmol), copper(I) iodide (180 mg, 0.94 mmol), lithium chloride (180 mg, 4.29 mmol) and tetrakis(triphenylphosphine) palladium (350 mg, 0.30 mmol). The reaction was stirred at reflux for 8 hrs, and it was cooled to room temperature. The mixture was diluted with dichloromethane and water. Combined organic layer was dried over sodium sulfate and evaporated. The residue was purified by chromatography on silica to give the title compound (180 mg, 27%). MS (ESI, m/e) [M+H]⁺ 646.3.

### Step 4: 6-(6-chloro-2,8-difluoro-4-((S)-3-methylmorpholino)quinazolin-7-yl)-5-iodo-N,N-bis(4-methoxybenzyl)-4-methylpyridin-2-amine

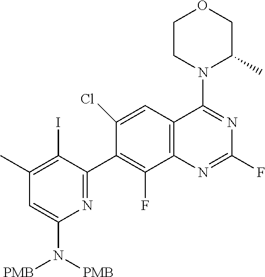

To a solution of (S)-6-(6-chloro-2,8-difluoro-4-(3-methylmorpholino)quinazolin-7-yl)-N,N-bis(4-methoxybenzyl)-4-methylpyridin-2-amine (120 mg, 0.19 mmol) in 20 mL N,N-dimethylformamide was added iodine (110 mg, 0.43 mmol), silver sulfate (20 mg, 0.06 mmol). The reaction was stirred at room temperature for 16 hrs, and the mixture was diluted with dichloromethane and water. Combined organic layer was dried over sodium sulfate and evaporated. The residue was purified by chromatography on silica to give the title compound (60 mg, 42%). MS (ESI, m/e) [M+H]⁺ 772.5.

### Step 5: 6-(6-chloro-2,8-difluoro-4-((S)-3-methylmorpholino)quinazolin-7-yl)-N,N-bis(4-methoxybenzyl)-4-methyl-5-(trifluoromethyl)pyridin-2-amine

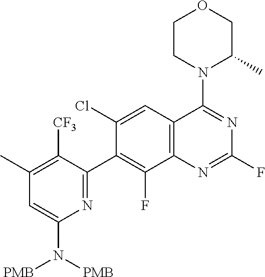

To a solution of 6-(6-chloro-2,8-difluoro-4-((S)-3-methylmorpholino)quinazolin-7-yl)-5-iodo-N,N-bis(4-methoxybenzyl)-4-methylpyridin-2-amine (60 mg, 0.08 mmol) in 5 mL N-Methyl-2-pyrrolidone was added methyl 2,2-difluoro-2-(fluorosulfonyl)acetate (150 mg, 0.79 mmol) and copper(I) iodide (45 mg, 0.24 mmol). The reaction was stirred at 90° C. for 2 hrs, and then cooled to room temperature. The mixture was diluted with dichloromethane and water, and combined organic layer was dried over sodium sulfate and evaporated. The residue was purified by Prep-TLC to give the title compound (30 mg, 54%). MS (ESI, m/e) [M+H]⁺ 714.3

### Step 6: 6-(6-chloro-8-fluoro-2-(((2R,7aS)-2-fluorotetrahydro-1H-pyrrolizin-7a(5H)-yl)methoxy)-4-((S)-3-methylmorpholino)quinazolin-7-yl)-N,N-bis(4-methoxybenzyl)-4-methyl-5-(trifluoromethyl)pyridin-2-amine

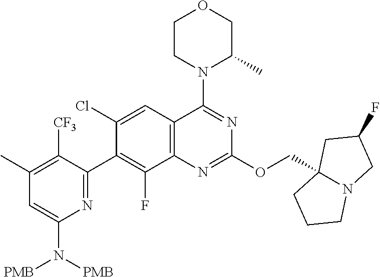

To a solution of ((2R,7aS)-2-fluorotetrahydro-1H-pyrrolizin-7a(5H)-yl)methanol hydrogen chloride (24 mg, 0.15 mmol) in 2 mL tetrahydrofuran was added sodium hydride (60%, 6 mg, 0.15 mmol) at 0° C., and the mixture was stirred at room temperature for 0.5 h. A solution of 6-(6-chloro-2,8-difluoro-4-((S)-3-methylmorpholino)quinazolin-7-yl)-N,N-bis(4-methoxybenzyl)-4-methyl-5-(trifluoromethyl)pyridin-2-amine (30 mg, 0.04 mmol) in 1 mL tetrahydrofuran was added dropwise. After addition, the reaction was stirred at room temperature for 0.5 h, and the mixture was diluted with dichloromethane and water. Combined organic layer was dried over sodium sulfate and evaporated. The residue was purified by Prep-TLC to give the title compound (18 mg, 50%). MS (ESI, m/e) [M+H]⁺ 853.3

### Step 7: 6-(6-chloro-8-fluoro-2-(((2R,7aS)-2-fluorotetrahydro-1H-pyrrolizin-7a(5H)-yl)methoxy)-4-((S)-3-methylmorpholino)quinazolin-7-yl)-4-methyl-5-(trifluoromethyl)pyridin-2-amine

A solution of 6-(6-chloro-8-fluoro-2-(((2R,7aS)-2-fluorotetrahydro-1H-pyrrolizin-7a(5H)-yl)methoxy)-4-((S)-3-methylmorpholino)quinazolin-7-yl)-N,N-bis(4-methoxybenzyl)-4-methyl-5-(trifluoromethyl)pyridin-2-amine (18 mg, 0.02 mmol) in trifluoroacetic acid (1 mL) was stirred at room temperature for 16 hrs, then it was stirred at 40° C. for 8 hrs. Then, the solvents was evaporated, and pH was adjusted to 11 with aq sodium carbonate solution. The mixture was diluted with dichloromethane and water, and combined organic layer was dried over sodium sulfate and evaporated. The residue was purified by Prep-HPLC to give the title compound (5 mg, 38%). ¹H NMR (500 MHz, CD₃OD) δ 7.81 (s, 1H), 6.60 (s, 1H), 5.39-5.23 (m, 1H), 4.72-4.62 (m, 1H), 4.33-4.06 (m, 3H), 3.99-3.92 (m, 1H), 3.88-3.69 (m, 4H), 3.06-2.99 (m, 1H), 2.47 (s, 3H), 2.28-1.85 (m, 6H), 1.55-1.48 (m, 3H). MS (ESI, m/e) [M+H]⁺ 613.4.

### Example 106: 8-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methyl-1-oxa-3,8-diazaspiro[4.5]decan-2-one

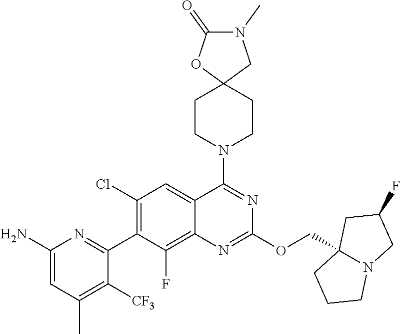 [SMILES: Cc1cc(N)nc(-c2c(Cl)cc3c(N4CCC5(CC4)CN(C)C(=O)O5)nc(OC[C@@]45CCCN4C[C@H](F)C5)nc3c2F)c1C(F)(F)F]

### Step 1: 8-(7-bromo-2,6-dichloro-8-fluoroquinazolin-4-yl)-3-methyl-1-oxa-3,8-diazaspiro[4.5]decan-2-one

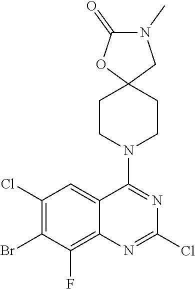 [SMILES: CN1CC2(CCN(c3nc(Cl)nc4c(F)c(Br)c(Cl)cc34)CC2)OC1=O]

A mixture of 7-bromo-2,4,6-trichloro-8-fluoroquinazoline (1.27 g, 3.9 mmol), 3-methyl-1-oxa-3,8-diazaspiro[4.5]decan-2-one hydrogen chloride (800 mg, 3.9 mmoL) in DCM (50 mL) and DIPEA (6 mL) was stirred for 2 hours at 0° C. to room temperature. After completion, solvents were evaporated and the crude product was purified by silica column (eluting with PE/EtOAc=1/4) to afford the title product (1.4 g). MS (ESI, m/e) [M+H]⁺ 463.3.

### Step 2: 8-(7-bromo-6-chloro-2,8-difluoroquinazolin-4-yl)-3-methyl-1-oxa-3,8-diazaspiro[4.5]decan-2-one

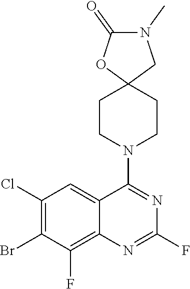 [SMILES: CN1CC2(CCN(c3nc(F)nc4c(F)c(Br)c(Cl)cc34)CC2)OC1=O]

To a mixture of 8-(7-bromo-2,6-dichloro-8-fluoroquinazolin-4-yl)-3-methyl-1-oxa-3,8-diazaspiro[4.5]decan-2-one (1.4 g, 3.0 mmol) in DMSO (40 mL) was added KF (2 g) and the mixture was stirred for 5 hours at 100° C. After completion, the resulting mixture was cooled, poured into water and extracted with EtOAc. The organic layer was combined and concentrated. Then the residue was further purified by silica column (eluting with PE/EA=1/9) to give the title product (1 g). MS (ESI, m/e) [M+H]⁺ 447.2

### Step 3: 8-(7-(6-(bis(4-methoxybenzyl)amino)-4-methylpyridin-2-yl)-6-chloro-2,8-difluoroquinazolin-4-yl)-3-methyl-1-oxa-3,8-diazaspiro[4.5]decan-2-one

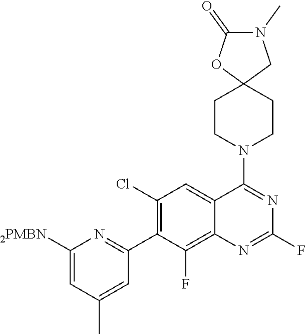

A mixture of 4-chloro-N,N-bis(4-methoxybenzyl)-4-methyl-6-(tributylstannyl)pyridin-2-amine (1.4 g, 2.2 mmol), 8-(7-bromo-6-chloro-2,8-difluoroquinazolin-4-yl)-3-methyl-1-oxa-3,8-diazaspiro[4.5]decan-2-one (500 mg, 1.1 mmol), Pd(PPh₃)₄ (387 mg, 0.3 mmol), CuI (225 mg), LiCl (170 mg) in dioxane (40 mL) was stirred for 14 hours at 110° C. After completion, the resulting mixture was directly concentrated. Then the crude product was further purified by silica column (eluting with PE/EtOAc=1/4) to give the title product (500 mg). MS (ESI, m/e) [M+H]⁺ 715.4.

### Step 4: 8-(7-(6-(bis(4-methoxybenzyl)amino)-3-iodo-4-methylpyridin-2-yl)-6-chloro-2,8-difluoroquinazolin-4-yl)-3-methyl-1-oxa-3,8-diazaspiro[4.5]decan-2-one

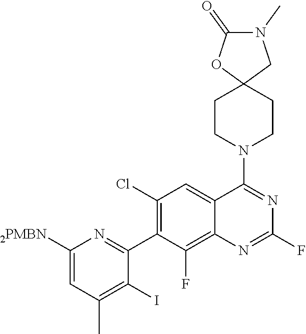

A mixture of 8-(7-(6-(bis(4-methoxybenzyl)amino)-4-methylpyridin-2-yl)-6-chloro-2,8-difluoroquinazolin-4-yl)-3-methyl-1-oxa-3,8-diazaspiro[4.5]decan-2-one (500 mg, 0.7 mmol), Ag₂SO₄ (100 mg, 0.3 mmol), I₂ (500 mg, 2 mmol) in DMF (50 mL) was stirred for 1 h at room temperature. After completion, the resulting mixture was directly poured into water and extracted with EtOAc. Then the crude product was further purified by silica column to give the title product (300 mg). MS (ESI, m/e) [M+H]⁺841.3.

### Step 5: 8-(7-(6-(bis(4-methoxybenzyl)amino)-4-methyl-3-(trifluoromethyl)pyridin-2-yl)-6-chloro-2,8-difluoroquinazolin-4-yl)-3-methyl-1-oxa-3,8-diazaspiro[4.5]decan-2-one

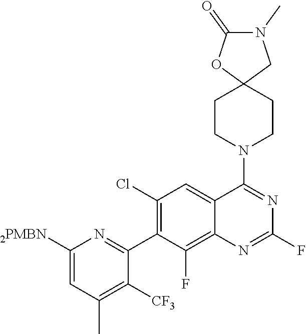

A mixture of 8-(7-(6-(bis(4-methoxybenzyl)amino)-3-iodo-4-methylpyridin-2-yl)-6-chloro-2,8-difluoroquinazolin-4-yl)-3-methyl-1-oxa-3,8-diazaspiro[4.5]decan-2-one (300 mg, 0.36 mmol), CuI (300 mg, 1.6 mmol), methyl 2,2-difluoro-2-(fluorosulfonyl)acetate (1.5 mL) in NMP (20 mL) was stirred for 1 hours at 90° C. After completion, the resulting mixture was directly poured into water and extracted with EtOAc. Then the crude product was further purified by silica column to give the title product (150 mg). MS (ESI, m/e) [M+H]⁺783.4.

### Step 6: 8-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methyl-1-oxa-3,8-diazaspiro[4.5]decan-2-one

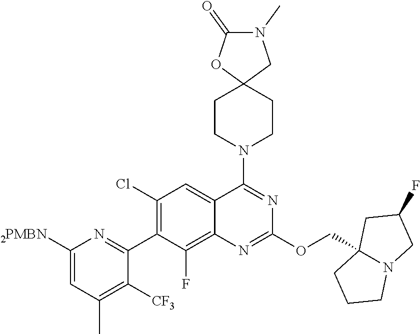

To a solution of ((2S,4R)-4-fluoro-1-methylpyrrolidin-2-yl)methanol (150 mg, 0.94 mmol) in THF (20 mL) was added NaH (50 mg) and the mixture was stirred for 30 mins at room temperature. Then, 8-(7-(6-(bis(4-methoxybenzyl)amino)-4-methyl-3-(trifluoromethyl)pyridin-2-yl)-6-chloro-2,8-difluoroquinazolin-4-yl)-3-methyl-1-oxa-3,8-diazaspiro[4.5]decan-2-one (150 mg, 0.19 mmol) was added. The reaction mixture was stirred for 4 hrs at 50° C. After completion, the reaction was quenched by H₂O. Solvents were evaporated and the crude product was purified by silica column (eluting with DCM/MeOH=4/1) to give the title product (120 mg). MS (ESI, m/e) [M+H]⁺ 922.3.

### Step 7: 8-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methyl-1-oxa-3,8-diazaspiro[4.5]decan-2-one

A mixture of 8-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methyl-1-oxa-3,8-diazaspiro[4.5]decan-2-one (120 mg, 0.13 mmol) in TFA (4 mL) was stirred for 3 hrs at 50° C. After completion, the resulting mixture was directly concentrated and pH of the residue was adjusted to 9 with DIPEA. Then the residues were purified by C18 column (eluting with H₂O/CH₃CN=3/1) to afford the title product (14 mg). ¹H NMR (500 MHz, CD₃OD) δ 7.88 (s, 1H), 6.60 (s, 1H), 5.50-5.31 (m, 1H), 4.49-4.35 (m, 2H), 4.28-4.10 (m, 2H), 3.87-3.75 (m, 2H), 3.65-3.38 (m, 5H), 3.23-3.15 (m, 1H), 2.89 (s, 3H), 2.51-1.98 (m, 13H). MS (ESI, m/e) [M+H]⁺ 682.6.

### Example 107: 1-(7-(2-amino-6-methyl-5-(trifluoromethyl)pyrimidin-4-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

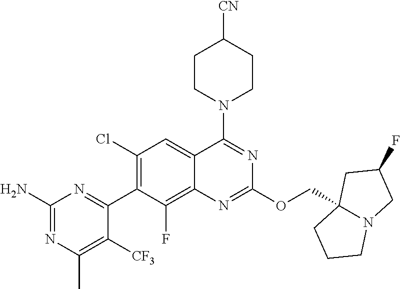 [SMILES: Cc1nc(N)nc(-c2c(Cl)cc3c(N4CCC(C#N)CC4)nc(OC[C@@]45CCCN4C[C@H](F)C5)nc3c2F)c1C(F)(F)F]

### Step 1: 1-(7-(6-(bis(4-methoxybenzyl)amino)-4-methoxypyridin-2-yl)-6-chloro-2,8-difluoroquinazolin-4-yl)piperidine-4-carbonitrile

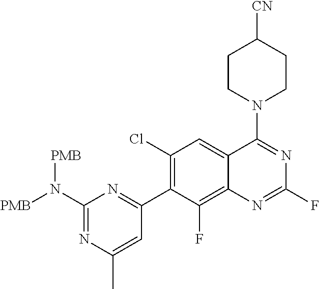

A mixture of 1-(7-bromo-6-chloro-2,8-difluoroquinazolin-4-yl)piperidine-4-carbonitrile (400 mg, 1.036 mmol), 4-bromo-N,N-bis(4-methoxybenzyl)-6-methylpyrimidin-2-amine (884 mg, 2.072 mmol), Pd(PPh₃)₄ (120 mg, 0.104 mmol), Pd(PPh₃)₂Cl₂ (72 mg, 0.104 mmol) and Bu₆Sn₂ (1.2 g, 2.07 mmol) was stirred at 100° C. overnight. The mixture was concentrated in vacuo, and the residue was purified by silica gel column chromatography (PE/EA=4/1) to give the title product (329 mg, crude). MS (ESI, m/e) [M+H]⁺ 656.6.

### Step 2: 1-(7-(2-(bis(4-methoxybenzyl)amino)-5-iodo-6-methylpyrimidin-4-yl)-6-chloro-2,8-difluoroquinazolin-4-yl)piperidine-4-carbonitrile

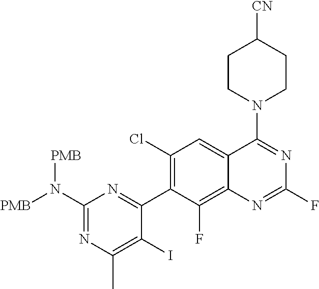

Into a 50 mL round bottom flask, 1-(7-(6-(bis(4-methoxybenzyl)amino)-4-methylpyridin-2-yl)-2,8-difluoroquinazolin-4-yl)piperidine-4-carbonitrile (130 mg, 0.387 mmol) and NIS (261 mg, 1.161 mmol) was added, and the mixture was cooled to 0° C. Then, HOAc (10 mL) was added. The mixture was stirred at 0° C. for 15 min-20 min. Then, the mixture was diluted with EtOAc, quenched with Na₂S₂O₃ solution, and extracted with EtOAc. The combined organic layer was concentrated under vacuum and purified by silica column chromatography (PE/EA=4/1) to give the title product (40 mg, 33%). MS (ESI, m/e) [M+H]⁺ 782.3.

### Step 3: 1-(7-(2-(bis(4-methoxybenzyl)amino)-6-methyl-5-(trifluoromethyl)pyrimidin-4-yl)-6-chloro-2,8-difluoroquinazolin-4-yl)piperidine-4-carbonitrile

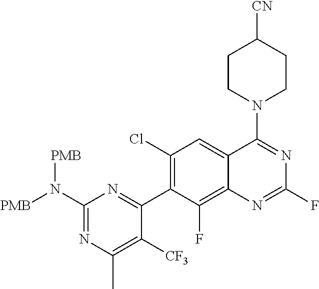

A mixture of 1-(7-(2-(bis(4-methoxybenzyl)amino)-5-iodo-6-methylpyrimidin-4-yl)-6-chloro-2,8-difluoroquinazolin-4-yl)piperidine-4-carbonitrile (40 mg, 0.055 mmol), CuI (21 mg, 0.11 mmol) and methyl 2,2-difluoro-2-(fluorosulfonyl)acetate (53 mg, 0.276 mmol) in DMA (5 mL) was stirred at 120° C. for 2 hrs. The reaction mixture was cooled to room temperature, and the solvent was extracted with EtOAc (20 mL*3). The combined organic phase was washed with brine (50 mL), and dried over Na₂SO₄. The mixture was concentrated in vacuo to give the title product, which was used directly without further purification (90 mg, crude). MS (ESI, m/e) [M+H]⁺ 724.5.

### Step 5: 1-(7-(2-(bis(4-methoxybenzyl)amino)-6-methyl-5-(trifluoromethyl)pyrimidin-4-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

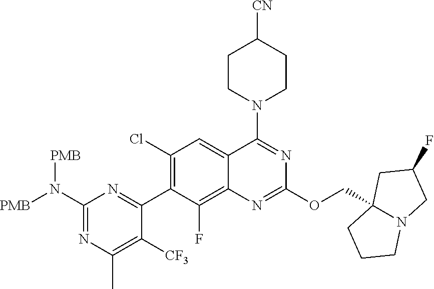

To a solution of ((2R,7aS)-2-fluorotetrahydro-1H-pyrrolizin-7a(5H)-yl)methanol (20 mg, 0.124 mmol) in THF (10 mL) was added NaH (7.5 mg, 0.186 mmol) at 0° C. The mixture was stirred at 0° C. for 30 mins. Then, 1-(7-(2-(bis(4-methoxybenzyl)amino)-6-methyl-5-(trifluoromethyl)pyrimidin-4-yl)-6-chloro-2,8-difluoroquinazolin-4-yl)piperidine-4-carbonitrile (90 mg, 0.124 mmol) was added. The mixture was stirred from 0° C. to room temperature, and then stirred for another 3 hrs at room temperature. The mixture was quenched by water (1 mL) and concentrated in vacuo. The residue was purified by silica gel column chromatography (PE/EA=1/4) to give the title product (35 mg, 32%). MS (ESI, m/e) [M+H]⁺ 863.7.

### Step 6: 1-(7-(6-amino-4-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

A mixture of 1-(7-(2-(bis(4-methoxybenzyl)amino)-6-methyl-5-(trifluoromethyl)pyrimidin-4-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile (35 mg, 0.040 mmol) and TFA (3 ml) was stirred at 50° C. for 2 hrs, and then stirred at 60° C. overnight. Solvent was removed in vacuo to give the crude product which was purified by prep-HPLC to give the title product (3.63 mg). ¹H NMR (500 MHz, DMSO-d₆) δ 7.85 (s, 1H), 7.80-7.58 (m, 2H), 5.38-5.17 (m, 1H), 4.14-3.90 (m, 4H), 3.66-3.50 (m, 2H), 3.24 (s, 1H), 3.12-2.97 (m, 3H), 2.87-2.79 (m, 1H), 2.16-1.68 (m, 10H). MS (ESI, m/e) [M+H]⁺ 623.4.

### Example 108: 1-(7-(6-amino-4-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

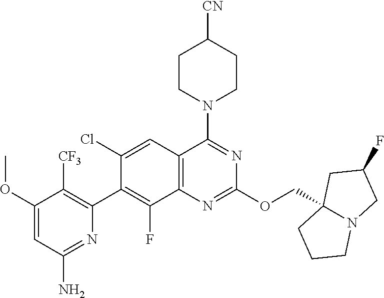 [SMILES: COc1cc(N)nc(-c2c(Cl)cc3c(N4CCC(C#N)CC4)nc(OC[C@@]45CCCN4C[C@H](F)C5)nc3c2F)c1C(F)(F)F]

### Step 1: 6-bromo-4-methoxy-N,N-bis(4-methoxybenzyl)pyridin-2-amine

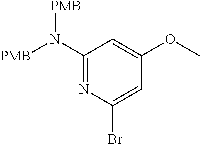

To the solution of 6-bromo-4-methoxypyridin-2-amine (1 g, 4.92 mmol) in DMF (20 mL) was added NaH (0.43 g, 10.8 mmol) dropwise at 0° C., and the mixture was stirred at 0° C. for another 30 min. Then, PMBCl (1.7 g, 10.8 mmol) was added, and the mixture was stirred at room temperature overnight. The mixture was diluted with water, filtered and solid was collected to give the title product (2 g, 92%). MS (ESI, m/e) [M+H]⁺ 443.1.

### Step 2: 1-(7-(6-(bis(4-methoxybenzyl)amino)-4-methoxypyridin-2-yl)-6-chloro-2,8-difluoroquinazolin-4-yl)piperidine-4-carbonitrile

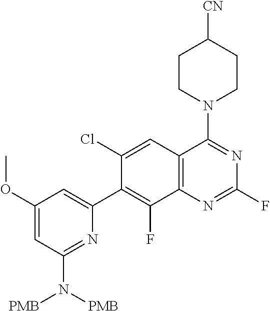

To a mixture of 1-(7-bromo-6-chloro-2,8-difluoroquinazolin-4-yl)piperidine-4-carbonitrile (0.4 g, 0.76 mmol), 6-bromo-4-methoxy-N,N-bis(4-methoxybenzyl)pyridin-2-amine (0.5 g, 1.14 mmol), Pd(PPh₃)₄ (87.8 mg, 0.076 mmol), Pd(PPh₃)₂Cl₂ (53 mg, 0.076 mmol) and Bu₆Sn₂ (0.66 g, 1.14 mmol) was stirred at 90° C. overnight. The reaction mixture was cooled to room temperature and filtered. Filtrate was concentrated, diluted with H₂O (10 mL), then extracted with EtOAc (20 mL×3). The combined organic layer was dried over Na₂SO₄, filtered and concentrated to give crude product, which was purified by silica gel chromatography (PE:EA=20:1-1:1) to give the title product (0.5 g, 72%). MS (ESI, m/e) [M+H]⁺ 671.2.

### Step 3: 1-(7-(6-(bis(4-methoxybenzyl)amino)-3-iodo-4-methoxypyridin-2-yl)-6-chloro-2,8-difluoroquinazolin-4-yl)piperidine-4-carbonitrile

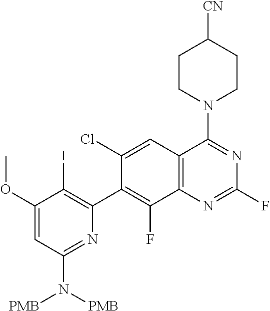

To a stirred solution of 1-(7-(6-(bis(4-methoxybenzyl)amino)-4-methoxypyridin-2-yl)-6-chloro-2,8-difluoroquinazolin-4-yl)piperidine-4-carbonitrile (0.47 g, 0.7 mmol) in DMF/AcOH=4:1 (5 mL) was added NIS (0.47 g, 2.1 mmol) in dropwise at 0° C. The mixture was stirred at room temperature for 2 hrs. The solvent was removed by reduced pressure, and the residue was diluted with saturated NaHCO₃ solution (100 mL), and extracted with EtOAc (20 mL*3). The combined organic phase was washed with brine (20 mL*2) and dried over Na₂SO₄. Filtered off the solid and concentrated to give crude product, which was purified by silica gel chromatography (PE:EA=20:1-1:1) to give the title product (0.45 g, 81%). MS (ESI, m/e) [M+H]⁺ 797.1.

### Step 4: 1-(7-(6-(bis(4-methoxybenzyl)amino)-4-methoxy-3-(trifluoromethyl)pyridin-2-yl)-6-chloro-2,8-difluoroquinazolin-4-yl)piperidine-4-carbonitrile

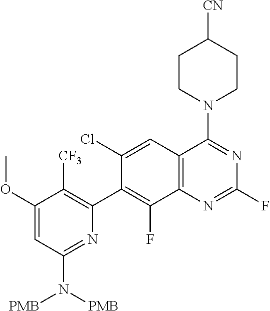

A mixture of 1-(7-(6-(bis(4-methoxybenzyl)amino)-3-iodo-4-methoxypyridin-2-yl)-6-chloro-2,8-difluoroquinazolin-4-yl)piperidine-4-carbonitrile (0.4 g, 0.5 mmol), CuI (0.8 g, 4.2 mmol) and methyl 2,2-difluoro-2-(fluorosulfonyl)acetate (1.6 g, 8.3 mmol) in DMA (10 mL) was stirred at 120° C. for 2 hrs. The reaction mixture was cooled to room temperature, and the solvent was removed in vacuo to give the crude product, which was purified by silica gel chromatography (DCM:MeOH=50:1-10:1) to give the title product (0.2 g, 48%). MS (ESI, m/e) [M+H]⁺ 739.2.

### Step 5: 1-(7-(6-(bis(4-methoxybenzyl)amino)-4-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

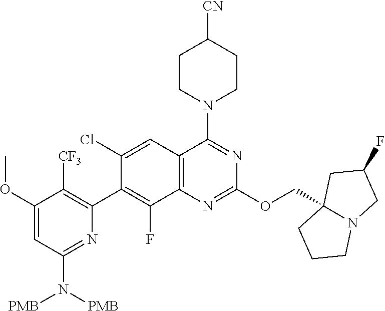

To the solution of 1-(7-(6-(bis(4-methoxybenzyl)amino)-4-methoxy-3-(trifluoromethyl)pyridin-2-yl)-6-chloro-2,8-difluoroquinazolin-4-yl)piperidine-4-carbonitrile (100 mg, 0.135 mmol) and ((2R,7aS)-2-fluorotetrahydro-1H-pyrrolizin-7a(5H)-yl)methanol (21.6 mg, 0.135 mmol) in THF (10 mL) was added NaH (8 mg, 0.2 mmol) at 0° C., The mixture was stirred at room temperature for 3 hrs. The mixture was diluted with ice-water, filtered to collect solid as crude product, which was purified by silica gel chromatography (DCM:MeOH=50:1-10:1) to give the title product (100 mg, 90%). MS (ESI, m/e) [M+H]⁺ 878.3.

### Step 6: 1-(7-(6-amino-4-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

To a mixture of 1-(7-(6-(bis(4-methoxybenzyl)amino)-4-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile (50 mg, 0.057 mmol) in CH₂Cl₂ (5 mL) was added TFA (1 mL) drop-wise. The mixture was stirred at RT for 2 hrs. The solvent was removed in vacuo to give the crude product which was purified by prep-HPLC to give the title product (10 mg, 36%). ¹H NMR (500 MHz, DMSO-d₆) δ 7.79 (s, 1H), 6.85 (s, 2H), 6.22 (s, 1H), 5.37-5.17 (m, 1H), 4.12-3.85 (m, 8H), 3.65-3.55 (m, 2H), 3.28-3.18 (m, 1H), 3.09-3.02 (m, 2H), 2.90-2.75 (m, 1H), 2.19-1.70 (m, 10H). MS (ESI) m/e [M+H]⁺=638.4.

### Example 109: 7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-N,N-dimethylquinazolin-4-amine

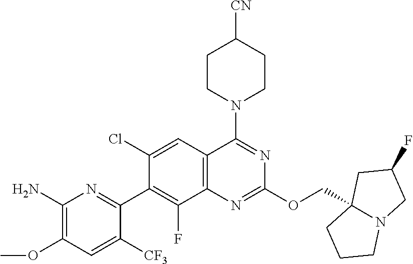 [SMILES: COc1cc(C(F)(F)F)c(-c2c(Cl)cc3c(N4CCC(C#N)CC4)nc(OC[C@@]45CCCN4C[C@H](F)C5)nc3c2F)nc1N]

### Step 1: 6-bromo-3-methoxy-N,N-bis(4-methoxybenzyl)pyridin-2-amine

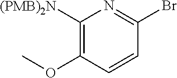

To a solution of 6-bromo-3-methoxypyridin-2-amine (2.0 g, 15.2 mmol) in DMF (40 mL) was added NaH (1.0 g, 25 mmol) slowly at 0° C., the reaction mixture was stirred at 0° C. for 0.5 hours, then PMBCl (3.3 g, 21 mmol) was added. The reaction mixture was allowed to be warmed to room temperature for 16 hours. EtOAc (200 mL) was added, organic layer was washed with water and brine, dried over Na₂SO₄. The filtrate was concentrated and purified by column chromograph (Hexane/EtOAc=4/1) to give the title product (3.7 g, 83%). MS (ESI, m/e) [M+H]⁺ 443.2.

### Step 2: 1-(7-(6-(bis(4-methoxybenzyl)amino)-5-methoxypyridin-2-yl)-6-chloro-2,8-difluoroquinazolin-4-yl)piperidine-4-carbonitrile

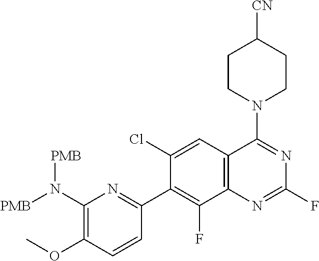

The mixture of 1-(7-bromo-6-chloro-2,8-difluoroquinazolin-4-yl)piperidine-4-carbonitrile (1.0 g, 2.6 mmol), 6-bromo-3-methoxy-N,N-bis(4-methoxybenzyl)pyridin-2-amine (1.5 g, 3.4 mmol), Pd(PPh₃)₂Cl₂ (150 mg, 0.21 mmol), Pd(PPh₃)₄ and Bu₆Sn₂ (1.8 g, 3.1 mmol) in dioxane (25 mL) was stirred at rt for 1 hr and then heated to 100° C. for 16 hrs. The mixture was concentrated and purified by column chromograph (Hexane/EtOAc=1/1) to give the title product (530 mg, 30%). MS (ESI, m/e) [M+H]⁺ 617.6.

### Step 3: 1-(7-(6-(bis(4-methoxybenzyl)amino)-3-iodo-5-methoxypyridin-2-yl)-6-chloro-2,8-difluoroquinazolin-4-yl)piperidine-4-carbonitrile

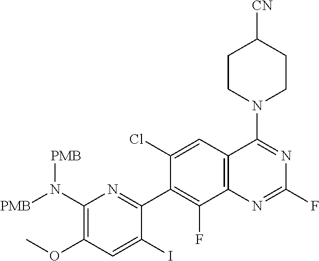

To a solution of 1-(7-(6-(bis(4-methoxybenzyl)amino)-5-methoxypyridin-2-yl)-6-chloro-2,8-difluoroquinazolin-4-yl)piperidine-4-carbonitrile (500 mg, 0.75 mmol) in HOAc/DMF (2 mL/10 mL) was added NIS (500 mg, 2.22 mmol) in portions at rt, and the mixture was stirred at rt for 6 hrs. EtOAc (50 mL) was added, and the organic phase was washed with water (30 mL×2) and brine (30 mL×2), dried over Na₂SO₄, concentrated and purified by column chromograph (Hexane/EtOAc=1/2) to give the title product (160 mg, 27%). MS (ESI, m/e) [M+H]⁺ 797.4.

### Step 4: 1-(7-(6-(bis(4-methoxybenzyl)amino)-5-methoxy-3-(trifluoromethyl)pyridin-2-yl)-6-chloro-2,8-difluoroquinazolin-4-yl)piperidine-4-carbonitrile

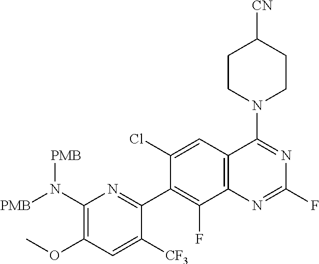

A mixture of 1-(7-(6-(bis(4-methoxybenzyl)amino)-3-iodo-5-methoxypyridin-2-yl)-6-chloro-2,8-difluoroquinazolin-4-yl)piperidine-4-carbonitrile (160 mg, 0.2 mmol), CuI (190 mg, 1.0 mmol) and methyl 2,2-difluoro-2-(fluorosulfonyl)acetate (300 mg, 1.56 mmol) in DMA (6 mL) was heated to 140° C. for 2 hrs. Cooling the reaction to room temperature, diluted with EtOAc (50 mL), washed with water (30 mL×2) and brine (30 mL×2), the resulting organic phase was dried over Na₂SO₄, concentrated and purified by column chromograph (Hexane/EtOAc=1/2) to give the title product (50 mg, 34%). MS (ESI, m/e) [M+H]⁺ 739.4.

### Step 5: 1-(7-(6-(bis(4-methoxybenzyl)amino)-5-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

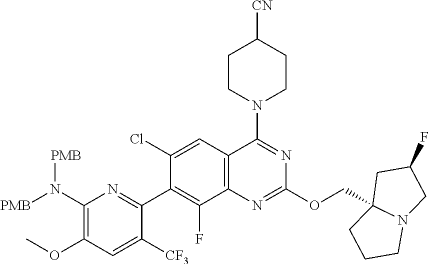

To a solution of ((2R,7aS)-2-fluorotetrahydro-1H-pyrrolizin-7a(5H)-yl)methanol (50 mg, 0.31 mmol) in THF was added NaH (10 mg, 0.25 mmol, 60%) in portions at 0° C., and the mixture was stirred at 0° C. for 30 min. 1-(7-(6-(bis(4-methoxybenzyl)amino)-5-methoxy-3-(trifluoromethyl)pyridin-2-yl)-6-chloro-2,8-difluoroquinazolin-4-yl)piperidine-4-carbonitrile (50 mg, 0.068 mmol) was added to the mixture in portions at 0° C. The mixture was stirred at rt for another 2 hrs. The reaction was quenched with aq. Sat. NH₄Cl solution, extracted with EtOAc for 3 times. The combined organic phase was dried over Na₂SO₄, concentrated and purified by column chromograph (DCM/MeOH=10/1) to give the title product (35 mg, 57%). MS (ESI, m/e) [M+H]⁺ 878.5.

### Step 6: 1-(7-(6-amino-5-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile

A mixture of 1-(7-(6-(bis(4-methoxybenzyl)amino)-5-methoxy-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)piperidine-4-carbonitrile (35 mg, 0.039 mmol) and TFA (3 mL) was stirred at 50° C. for 16 hrs. Concentrated and purified by Prep-HPLC to give the title product (7 mg, 28%). ¹H NMR (500 MHz, DMSO-d₆) δ 7.82 (s, 1H), 7.31 (s, 1H), 6.80 (s, 2H), 5.43-5.23 (m, 1H), 4.20-4.10 (m, 2H), 4.05-3.95 (m, 5H), 3.64-3.60 (m, 2H), 3.30-3.10 (m, 4H), 2.94-2.90 (m, 1H), 2.22-1.82 (m, 10H). MS (ESI, m/e) [M+H]⁺ 638.7.

### Example 110a (common intermediate): 7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-ol

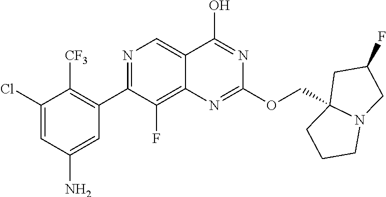 [SMILES: Nc1cc(Cl)c(C(F)(F)F)c(-c2ncc3c(O)nc(OC[C@@]45CCCN4C[C@H](F)C5)nc3c2F)c1]

### Step 1: 2,4,7-trichloro-8-fluoropyrido[4,3-d]pyrimidine

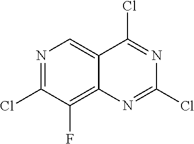 [SMILES: Fc1c(Cl)ncc2c(Cl)nc(Cl)nc12]

To a solution of 7-chloro-8-fluoropyrido[4,3-d]pyrimidine-2,4-diol (10 g, 39.8 mmol) in 200 mL POCl₃ was added DIPEA (10.0 mL), and the mixture was stirred at 100° C. for 4 hrs. Then, the mixture was cooled and concentrated to give a brown oil. The oil was dissolved in DCM and the solution was washed with ice-water twice. The organic layer was concentrated and purified by silica gel column with PE:EtOAc=3:1 to afford the title compound (7 g, 60%). MS (ESI, m/e) [M+H]⁺ 252.

### Step 2: 4-(benzyloxy)-2,7-dichloro-8-fluoropyrido[4,3-d]pyrimidine

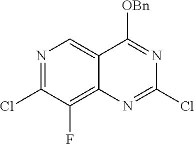 [SMILES: Fc1c(Cl)ncc2c(OCc3ccccc3)nc(Cl)nc12]

To a solution of 2,4,7-trichloro-8-fluoropyrido[4,3-d]pyrimidine (7 g, 28 mmol) in 100 mL of 1,4-dioxane was added benzyl alcohol (6 g, 56 mmol), DIPEA (10.8 g, 84 mmol), and molecular sieves (4 A°, 10 g). The mixture was stirred at 60° C. for 16 hrs. Then, it was diluted with water, and combined organic layer was dried over sodium sulfate. Solvent was evaporated and the residue was purified by chromatography column on silica to give the title compound (4 g, 78%). MS (ESI, m/e) [M+H]⁺ 324.

### Step 3: 4-(benzyloxy)-7-chloro-8-fluoro-2-(((2R,7aS)-2-fluorotetrahydro-1H-pyrrolizin-7a(5H)-yl)methoxy)pyrido[4,3-d]pyrimidine

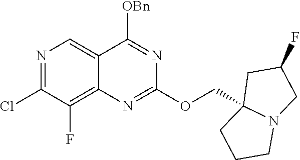 [SMILES: Fc1c(Cl)ncc2c(OCc3ccccc3)nc(OC[C@@]34CCCN3C[C@H](F)C4)nc12]

To a solution of 4-(benzyloxy)-2,7-dichloro-8-fluoropyrido[4,3-d]pyrimidine (4 g, 12.38 mmol) in 100 mL of 1,4-dioxane was added ((2R,7aS)-2-fluorotetrahydro-1H-pyrrolizin-7a(5H)-yl)methanol (3.9 g, 24.76 mmol), DIPEA (4.8 g, 37.14 mmol) and molecular sieves (4A°, 10 g). The mixture was stirred at 80° C. for 16 hrs. The mixture was diluted with water, and combined organic layer was dried over sodium sulfate. Solvents were evaporated and the residue was purified by chromatography column on silica to give the title product (3.4 g, 62%). MS (ESI, m/e) [M+H]⁺ 447.

### Step 4: 3-(4-(benzyloxy)-8-fluoro-2-(((2R,7aS)-2-fluorotetrahydro-1H-pyrrolizin-7a(5H)-yl)methoxy)pyrido[4,3-d]pyrimidin-7-yl)-5-chloro-4-(trifluoromethyl)aniline

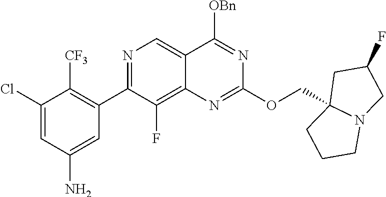 [SMILES: Nc1cc(Cl)c(C(F)(F)F)c(-c2ncc3c(OCc4ccccc4)nc(OC[C@@]45CCCN4C[C@H](F)C5)nc3c2F)c1]

To a 250 mL round-bottomed flask was added 4-(benzyloxy)-7-chloro-8-fluoro-2-(((2R,7aS)-2-fluorotetrahydro-1H-pyrrolizin-7a(5H)-yl)methoxy)pyrido[4,3-d]pyrimidine (3.4 g, 7.6 mmol), 3-chloro-5-(4,4,5,5-tetramethyl-1,3,2-dioxaborolan-2-yl)-4-(trifluoromethyl)aniline (2.9 g, 9.1 mmol), K₃PO₄ (3.2 g, 15.2 mmol), methanesulfonato(diadamantyl-n-butylphosphino)-2′-amino-1,1′-biphenyl-2-yl)palladium(II) dichloromethane adduct, min. 95% [cataCXium® A Palladacycle Gen. 3](0.55 g, 0.76 mmol), THF (120 mL) and H₂O (24 mL). The mixture was stirred at 70° C. for 3 hrs. The resulting cooled mixture was concentrated and purified by flash column chromatography (DCM/MeOH=30/1) to give the title product (2.8 g, 61%). MS (ESI, m/e) [M+H]⁺ 606.

### Step 5: 7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-ol

To a 250 mL round-bottomed flask was added 3-(4-(benzyloxy)-8-fluoro-2-(((2R,7aS)-2-fluorotetrahydro-1H-pyrrolizin-7a(5H)-yl)methoxy)pyrido[4,3-d]pyrimidin-7-yl)-5-chloro-4-(trifluoromethyl)aniline (2.8 g, 4.6 mmol) and TFA (50 mL), and the mixture was stirred at room temperature for overnight. The reaction mixture was concentrated and diluted with DCM. pH of the solution was adjusted to 7 with DIPEA. The mixture was then purified by reverse flash to give the title product (1.3 g, 55%). MS (ESI, m/e) [M+H]⁺ 516.2.

### Example 110: 7-(5-amino-3-chloro-2-(trifluoromethyl) phenyl)-N-cyclopropyl-8-fluoro-2-(((2R,7aS)-2-fluorotetrahydro-1H-pyrrolizin-7a(5H)-yl) methoxy) pyrido[4,3-d]pyrimidin-4-amine

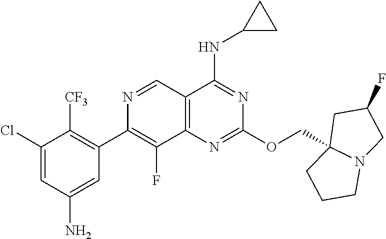 [SMILES: Nc1cc(Cl)c(C(F)(F)F)c(-c2ncc3c(NC4CC4)nc(OC[C@@]45CCCN4C[C@H](F)C5)nc3c2F)c1]

### Step 1: 7-(5-amino-3-chloro-2-(trifluoromethyl) phenyl)-N-cyclopropyl-8-fluoro-2-(((2R,7aS)-2-fluorotetrahydro-1H-pyrrolizin-7a(5H)-yl) methoxy) pyrido[4,3-d]pyrimidin-4-amine

To a mixture of 7-(5-amino-3-chloro-2-(trifluoromethyl) phenyl)-8-fluoro-2-(((2R,7aS)-2-fluorotetrahydro-1H-pyrrolizin-7a(5H)-yl) methoxy) pyrido[4,3-d]pyrimidin-4-ol (25 mg, 0.0485 mmol) in acetonitrile (5 mL) was added cyclopropanamine (7.2 mg, 0.126 mmol), BOP (43 mg, 0.097 mmol) and DIPEA (37.5 mg, 0.291 mmol) in step-wise. The mixture was stirred at 40° C. for overnight. The resulting mixture was concentrated to give a residue which was further purified by Prep-HPLC to give the title product (5.67 mg). ¹H NMR (500 MHz, MeOD) δ 9.08 (s, 1H), 6.90 (s, 1H), 6.48 (s, 1H), 5.50-5.35 (m, 1H), 4.58-4.44 (m, 2H), 3.67-3.43 (m, 3H), 3.25-3.12 (m, 2H), 2.57-1.96 (m, 6H), 0.97-0.92 (m, 2H), 0.81-0.75 (m, 2H). MS (ESI, m/e) [M+H]⁺ 555.3.

### Example 111: 7-(5-amino-3-chloro-2-(trifluoromethyl) phenyl)-N-cyclobutyl-8-fluoro-2-(((2R,7aS)-2-fluorotetrahydro-1H-pyrrolizin-7a(5H)-yl) methoxy) pyrido[4,3-d]pyrimidin-4-amine

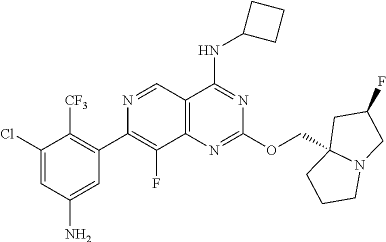 [SMILES: Nc1cc(Cl)c(C(F)(F)F)c(-c2ncc3c(NC4CCC4)nc(OC[C@@]45CCCN4C[C@H](F)C5)nc3c2F)c1]

Example 111 was prepared by similar procedure as described in Example 110 by replacing cyclopropanamine with cyclobutanamine to give the title product. ¹H NMR (500 MHz, CD₃OD) δ 9.17 (s, 1H), 6.91 (s, 1H), 6.49 (s, 1H), 5.48-5.34 (m, 1H), 4.50-4.39 (m, 2H), 3.64-3.42 (m, 3H), 3.24-3.14 (m, 1H), 2.51-2.25 (m, 7H), 2.17-1.82 (m, 5H). MS (ESI, m/e) [M+H]⁺ 569.4.

### Example 112: 7-(5-amino-3-chloro-2-(trifluoromethyl) phenyl)-N-(bicyclo [1.1.1]pentan-1-yl)-8-fluoro-2-(((2R,7aS)-2-fluorotetrahydro-1H-pyrrolizin-7a(5H)-yl) methoxy) pyrido[4,3-d]pyrimidin-4-amine

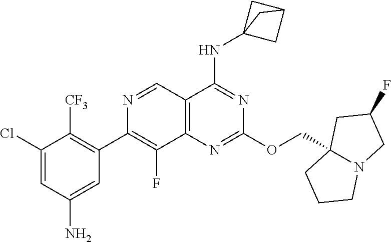 [SMILES: Nc1cc(Cl)c(C(F)(F)F)c(-c2ncc3c(NC45CC(C4)C5)nc(OC[C@@]45CCCN4C[C@H](F)C5)nc3c2F)c1]

Example 112 was prepared by similar procedure as described in Example 110 by replacing cyclopropanamine with bicyclo[1.1.1]pentan-1-amine to give the title product. ¹H NMR (500 MHz, CD₃OD) δ 9.10 (s, 1H), 6.90 (s, 1H), 6.49 (s, 1H), 5.47-5.36 (m, 1H), 4.50-4.37 (m, 2H), 3.67-3.42 (m, 3H), 3.24-3.15 (m, 1H), 2.62-2.31 (m, 9H), 2.28-1.96 (m, 4H). MS (ESI, m/e) [M+H]⁺ 581.4.

### Example 113: 3-chloro-5-(8-fluoro-2-(((2R,7aS)-2-fluorotetrahydro-1H-pyrrolizin-7a(5H)-yl) methoxy)-4-morpholinopyrido[4,3-d]pyrimidin-7-yl)-4-(trifluoromethyl)aniline

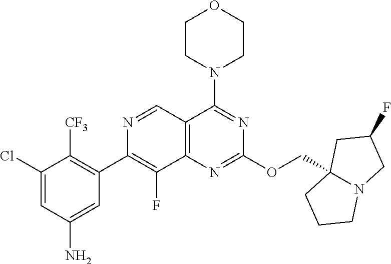 [SMILES: Nc1cc(Cl)c(C(F)(F)F)c(-c2ncc3c(N4CCOCC4)nc(OC[C@@]45CCCN4C[C@H](F)C5)nc3c2F)c1]

Example 113 was prepared by similar procedure as described in Example 110 by replacing cyclopropanamine with morpholine to give the title product. ¹H NMR (500 MHz, CD₃OD) δ 9.05 (s, 1H), 6.94-6.88 (m, 1H), 6.53-6.46 (m, 1H), 5.48-5.34 (m, 1H), 4.50-4.34 (m, 2H), 4.14-4.08 (m, 4H), 3.87-3.85 (m, 4H), 3.60-3.42 (m, 3H), 3.23-3.14 (m, 1H), 2.52-1.94 (m, 6H). MS (ESI, m/e) [M+H]⁺ 585.3.

### Example 114: 3-chloro-5-(8-fluoro-2-(((2R,7aS)-2-fluorotetrahydro-1H-pyrrolizin-7a(5H)-yl)methoxy)-4-((S)-3-methylmorpholino)pyrido[4,3-d]pyrimidin-7-yl)-4-(trifluoromethyl)aniline

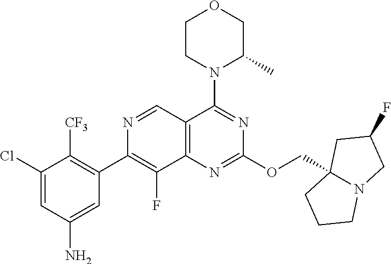 [SMILES: C[C@H]1COCCN1c1nc(OC[C@@]23CCCN2C[C@H](F)C3)nc2c(F)c(-c3cc(N)cc(Cl)c3C(F)(F)F)ncc12]

Example 114 was prepared by similar procedure as described in Example 110 by replacing cyclopropanamine with (S)-3-methylmorpholine to give the title product. ¹H NMR (500 MHz, CD₃OD) δ 9.02 (s, 1H), 6.98-6.84 (m, 1H), 6.54-6.47 (m, 1H), 5.52-5.32 (m, 1H), 4.95-4.92 (m, 1H), 4.55-4.38 (m, 3H), 4.05-3.53 (m, 8H), 3.29-3.24 (m, 1H), 2.59-1.99 (m, 6H), 1.65-1.55 (m, 3H). MS (ESI, m/e) [M+H]⁺ 599.2.

### Example 115: 3-chloro-5-(8-fluoro-2-(((2R,7aS)-2-fluorotetrahydro-1H-pyrrolizin-7a(5H)-yl)methoxy)-4-((R)-3-methylmorpholino)pyrido[4,3-d]pyrimidin-7-yl)-4-(trifluoromethyl)aniline

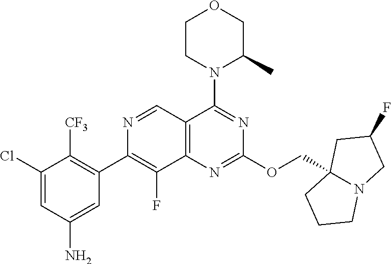 [SMILES: C[C@@H]1COCCN1c1nc(OC[C@@]23CCCN2C[C@H](F)C3)nc2c(F)c(-c3cc(N)cc(Cl)c3C(F)(F)F)ncc12]

Example 115 was prepared by similar procedure as described in Example 110 by replacing cyclopropanamine with (R)-3-methylmorpholine to give the title product. ¹H NMR (500 MHz, CD₃OD) δ 9.00 (s, 1H), 6.95-6.84 (m, 1H), 6.55-6.47 (m, 1H), 5.42-5.32 (m, 1H), 4.50-4.35 (m, 3H), 4.05-3.58 (m, 5H), 3.46-3.36 (m, 3H), 3.20-3.10 (m, 1H), 2.48-1.89 (m, 6H), 1.65-1.55 (m, 3H). MS (ESI, m/e) [M+H]⁺ 599.4.

### Example 116: 3-chloro-5-(4-((S)-3-ethylmorpholino)-8-fluoro-2-(((2R,7aS)-2-fluorotetrahydro-1H-pyrrolizin-7a(5H)-yl)methoxy)pyrido[4,3-d]pyrimidin-7-yl)-4-(trifluoromethyl)aniline

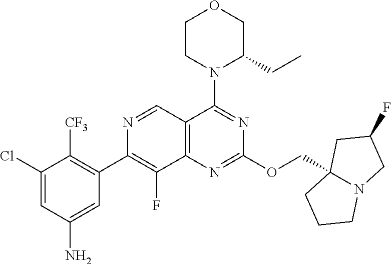 [SMILES: CC[C@H]1COCCN1c1nc(OC[C@@]23CCCN2C[C@H](F)C3)nc2c(F)c(-c3cc(N)cc(Cl)c3C(F)(F)F)ncc12]

Example 116 was prepared by similar procedure as described in Example 110 by replacing cyclopropanamine with (S)-3-ethylmorpholine to give the title product. ¹H NMR (500 MHz, CD₃OD) δ 9.02 (s, 1H), 6.95-6.86 (m, 1H), 6.60-6.41 (m, 1H), 5.50-5.32 (m, 1H), 4.47-4.26 (m, 3H), 4.01-3.92 (m, 3H), 3.82-3.49 (m, 6H), 3.24-3.17 (m, 1H), 2.50-2.01 (m, 8H), 1.05-0.94 (m, 3H). MS (ESI, m/e) [M+H]⁺ 613.2.

### Example 117: 3-chloro-5-(4-((R)-3-ethylmorpholino)-8-fluoro-2-(((2R,7aS)-2-fluorotetrahydro-1H-pyrrolizin-7a(5H)-yl)methoxy)pyrido[4,3-d]pyrimidin-7-yl)-4-(trifluoromethyl)aniline

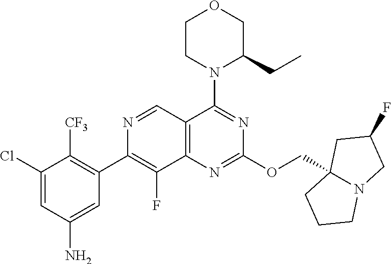 [SMILES: CC[C@@H]1COCCN1c1nc(OC[C@@]23CCCN2C[C@H](F)C3)nc2c(F)c(-c3cc(N)cc(Cl)c3C(F)(F)F)ncc12]

Example 117 was prepared by similar procedure as described in Example 110 by replacing cyclopropanamine with (R)-3-ethylmorpholine to give the title product. ¹H NMR (500 MHz, CD₃OD) δ 9.04 (s, 1H), 6.93-6.89 (m, 1H), 6.53-6.48 (m, 1H), 5.57-5.41 (m, 1H), 4.60-4.27 (m, 3H), 4.01-3.95 (m, 3H), 3.82-3.62 (m, 6H), 2.57-2.43 (m, 2H), 2.36-2.05 (m, 6H), 1.02-0.96 (m, 3H). MS (ESI, m/e) [M+H]⁺ 613.2.

### Example 118: 3-chloro-5-(4-((3S,5S)-3,5-dimethylmorpholino)-8-fluoro-2-(((2R,7aS)-2-fluorotetrahydro-1H-pyrrolizin-7a(5H)-yl) methoxy) pyrido[4,3-d]pyrimidin-7-yl)-4-(trifluoromethyl)aniline

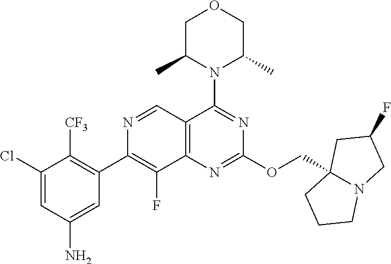 [SMILES: C[C@H]1COC[C@H](C)N1c1nc(OC[C@@]23CCCN2C[C@H](F)C3)nc2c(F)c(-c3cc(N)cc(Cl)c3C(F)(F)F)ncc12]

Example 118 was prepared by similar procedure as described in Example 110 by replacing cyclopropanamine with (3S,5S)-3,5-dimethylmorpholine to give the title product. ¹H NMR (500 MHz, CD₃OD) δ 9.12 (s, 1H), 6.95-6.89 (m, 1H), 6.54-6.47 (m, 1H), 5.54-5.40 (m, 1H), 4.60-4.50 (m, 2H), 4.26-4.18 (m, 2H), 4.12-4.04 (m, 2H), 3.73-3.53 (m, 5H), 2.63-2.00 (m, 6H), 1.27-1.18 (m, 6H). MS (ESI, m/e) [M+1]⁺ 613.5.

### Example 119: 3-chloro-5-(8-fluoro-2-(((2R,7aS)-2-fluorotetrahydro-1H-pyrrolizin-7a(5H)-yl)methoxy)-4-(2-methylmorpholino)pyrido[4,3-d]pyrimidin-7-yl)-4-(trifluoromethyl)aniline

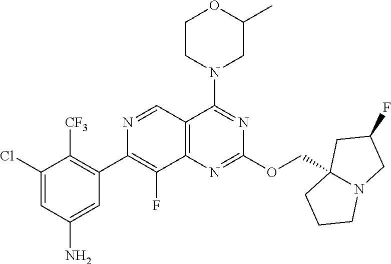 [SMILES: CC1CN(c2nc(OC[C@@]34CCCN3C[C@H](F)C4)nc3c(F)c(-c4cc(N)cc(Cl)c4C(F)(F)F)ncc23)CCO1]

Example 119 was prepared by similar procedure as described in Example 110 by replacing cyclopropanamine with 2-methylmorpholine to give the title product. ¹H NMR (500 MHz, CD₃OD) δ 9.13-8.96 (m, 1H), 7.05-6.84 (m, 1H), 6.59-6.42 (m, 1H), 5.53-5.36 (m, 1H), 4.60-4.49 (m, 3H), 4.48-4.41 (m, 1H), 4.07-3.98 (m, 1H), 3.81-3.73 (m, 2H), 3.63-3.56 (m, 3H), 3.29-3.22 (m, 3H), 2.56-1.99 (m, 6H), 1.29-1.21 (m, 3H). MS (ESI, m/e) [M+H]⁺ 599.2.

### Example 120: ((2S)-4-(7-(5-amino-3-chloro-2-(trifluoromethyl)phenyl)-8-fluoro-2-((2-fluorotetrahydro-1H-pyrrolizin-7a(5H)-yl)methoxy)pyrido[4,3-d]pyrimidin-4-yl)morpholin-2-yl)methanol

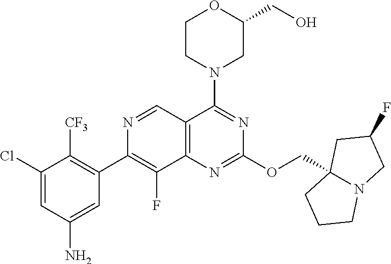 [SMILES: Nc1cc(Cl)c(C(F)(F)F)c(-c2ncc3c(N4CCO[C@H](CO)C4)nc(OC[C@@]45CCCN4C[C@H](F)C5)nc3c2F)c1]

Example 120 was prepared by similar procedure as described in Example 110 by replacing cyclopropanamine with (S)-morpholin-2-ylmethanol to give the title product. 1H NMR (500 MHz, CD₃OD) δ 9.07 (s, 1H), 6.91 (s, 1H), 6.50 (s, 1H), 5.50-5.34 (m, 1H), 4.68-4.32 (m, 4H), 4.08-4.05 (m, 1H), 3.84-3.44 (m, 9H), 3.28-3.34 (m, 1H), 2.51-1.92 (m, 6H). MS (ESI, m/e) [M+H]⁺ 615.5.

### Example 121: 1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbonitrile

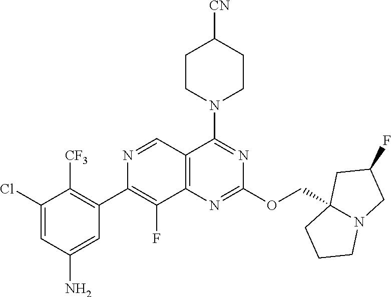 [SMILES: N#CC1CCN(c2nc(OC[C@@]34CCCN3C[C@H](F)C4)nc3c(F)c(-c4cc(N)cc(Cl)c4C(F)(F)F)ncc23)CC1]

Example 121 was prepared by similar procedure as described in Example 110 by replacing cyclopropanamine with piperidine-4-carbonitrile to give the title product. 1H NMR (500 MHz, CD₃OD) δ 9.02 (s, 1H), 6.91 (s, 1H), 6.50 (s, 1H), 5.52-5.34 (m, 1H), 4.54-4.25 (m, 4H), 3.97-3.86 (m, 2H), 3.62-3.47 (m, 3H), 3.26-3.18 (m, 2H), 2.57-1.98 (m, 10H). MS (ESI, m/e) [M+H]⁺ 608.2.

### Example 122: 1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-fluoropiperidine-4-carbonitrile

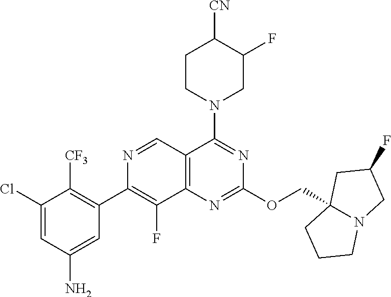 [SMILES: N#CC1CCN(c2nc(OC[C@@]34CCCN3C[C@H](F)C4)nc3c(F)c(-c4cc(N)cc(Cl)c4C(F)(F)F)ncc23)CC1F]

Example 122 was prepared by similar procedure as described in Example 110 by replacing cyclopropanamine with 3-fluoropiperidine-4-carbonitrile to give the title product. ¹H NMR (500 MHz, CD₃OD) δ 9.07 (s, 1H), 6.92 (s, 1H), 6.51 (s, 1H), 5.53-5.36 (m, 1H), 5.20-5.02 (m, 1H), 4.79-4.46 (m, 4H), 4.00-3.85 (m, 1H), 3.65-3.50 (m, 5H), 2.54-2.01 (m, 8H). MS (ESI, m/e) [M+H]⁺ 626.2.

### Example 123: 1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carbonitrile

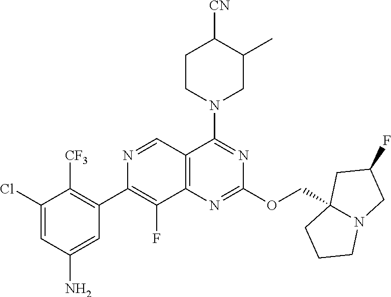 [SMILES: CC1CN(c2nc(OC[C@@]34CCCN3C[C@H](F)C4)nc3c(F)c(-c4cc(N)cc(Cl)c4C(F)(F)F)ncc23)CCC1C#N]

Example 123 was prepared by similar procedure as described in Example 110 by replacing cyclopropanamine with 3-methylpiperidine-4-carbonitrile to give the title product. ¹H NMR (500 MHz, CD₃OD) δ 9.04 (s, 1H), 6.91 (s, 1H), 6.50 (s, 1H), 5.58-5.40 (m, 1H), 4.71-4.55 (m, 3H), 3.75-3.60 (m, 6H), 2.88-2.05 (m, 10H), 1.30-1.18 (m, 3H). MS (ESI, m/e) [M+H]⁺ 622.2.

### Example 124: 1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-methylpiperidine-4-carbonitrile

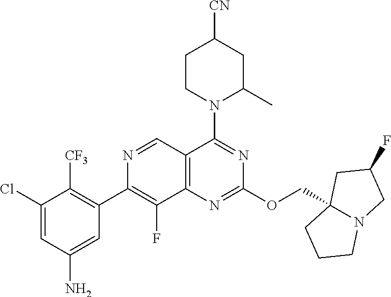 [SMILES: CC1CC(C#N)CCN1c1nc(OC[C@@]23CCCN2C[C@H](F)C3)nc2c(F)c(-c3cc(N)cc(Cl)c3C(F)(F)F)ncc12]

Example 124 was prepared by similar procedure as described in Example 110 by replacing cyclopropanamine with 2-methylpiperidine-4-carbonitrile to give the title product. ¹H NMR (500 MHz, CD₃OD) δ 8.97 (s, 1H), 6.98-6.86 (m, 1H), 6.64-6.45 (m, 1H), 5.58-5.41 (m, 1H), 5.20-5.06 (m, 1H), 4.65-4.48 (m, 3H), 3.89-3.60 (m, 4H), 3.39-3.33 (m, 2H), 2.68-1.99 (m, 10H), 1.56-1.50 (m, 3H). MS (ESI, m/e) [M+H]⁺ 622.2.

### Example 125: 2-(1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)acetonitrile

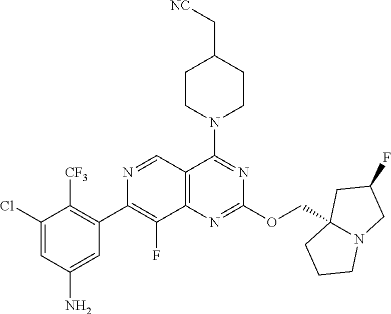 [SMILES: N#CCC1CCN(c2nc(OC[C@@]34CCCN3C[C@H](F)C4)nc3c(F)c(-c4cc(N)cc(Cl)c4C(F)(F)F)ncc23)CC1]

Example 125 was prepared by similar procedure as described in Example 110 by replacing cyclopropanamine with 2-(piperidin-4-yl)acetonitrile to give the title product. ¹H NMR (500 MHz, CD₃OD) δ 9.01 (s, 1H), 6.91 (s, 1H), 6.50 (s, 1H), 5.53-5.37 (m, 1H), 4.85-4.70 (m, 2H), 4.56-4.40 (m, 2H), 3.80-3.38 (m, 6H), 2.56-2.28 (m, 4H), 2.26-1.95 (m, 7H), 1.62-1.56 (m, 2H). MS (ESI, m/e) [M+H]⁺ 622.5.

### Example 126: 2-(1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile

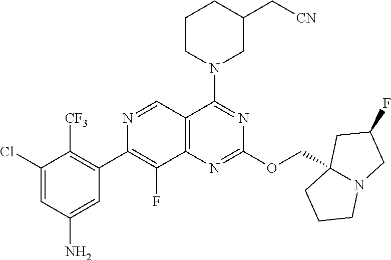 [SMILES: N#CCC1CCCN(c2nc(OC[C@@]34CCCN3C[C@H](F)C4)nc3c(F)c(-c4cc(N)cc(Cl)c4C(F)(F)F)ncc23)C1]

Example 126 was prepared by similar procedure as described in Example 110 by replacing cyclopropanamine with 2-(piperidin-3-yl)acetonitrile to give the title product. ¹H NMR (500 MHz, CD₃OD) δ 9.05 (s, 1H), 6.91 (s, 1H), 6.50 (s, 1H), 5.54-5.37 (m, 1H), 4.82-4.70 (m, 2H), 4.60-4.45 (m, 3H), 3.77-3.55 (m, 4H), 2.64-1.50 (m, 14H). MS (ESI, m/e) [M+H]⁺ 622.2.

### Example 127: 1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-ol

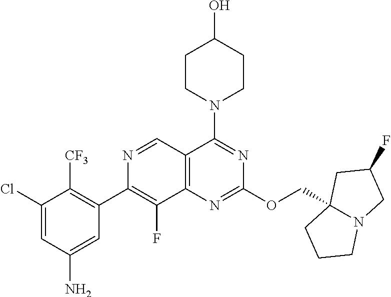 [SMILES: Nc1cc(Cl)c(C(F)(F)F)c(-c2ncc3c(N4CCC(O)CC4)nc(OC[C@@]45CCCN4C[C@H](F)C5)nc3c2F)c1]

Example 127 was prepared by similar procedure as described in Example 110 by replacing cyclopropanamine with piperidin-4-ol to give the title product. ¹H NMR (500 MHz, CD₃OD) δ 9.03 (s, 1H), 6.92 (s, 1H), 6.48 (s, 1H), 5.59-5.42 (m, 1H), 4.65-4.50 (m, 3H), 4.46-4.33 (m, 2H), 4.10-4.00 (m, 1H), 3.90-3.67 (m, 5H), 2.65-2.02 (m, 8H), 1.80-1.69 (m, 2H). MS (ESI, m/e) [M+H]⁺ 599.2.

### Example 128: (R)-1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol

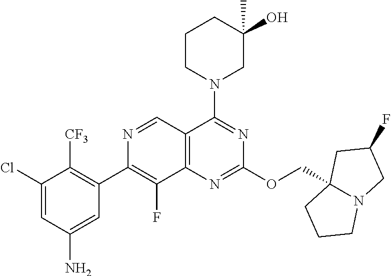 [SMILES: C[C@@]1(O)CCCN(c2nc(OC[C@@]34CCCN3C[C@H](F)C4)nc3c(F)c(-c4cc(N)cc(Cl)c4C(F)(F)F)ncc23)C1]

Example 128 was prepared by similar procedure as described in Example 110 by replacing cyclopropanamine with (3R)-3-methyl-piperidin-3-ol to give the title product. ¹H NMR (500 MHz, CD₃OD) δ 9.21 (s, 1H), 6.92 (s, 1H), 6.50 (s, 1H), 5.65-5.48 (m, 1H), 4.74-4.68 (m, 1H), 4.66-4.58 (m, 2H), 4.36-4.24 (m, 1H), 4.06-3.84 (m, 3H), 3.65-3.56 (m, 1H), 3.51-3.37 (m, 2H), 2.78-2.54 (m, 2H), 2.48-2.40 (m, 1H), 2.38-2.31 (m, 2H), 2.22-2.08 (m, 2H), 1.89-1.75 (m, 3H), 1.29 (s, 3H). MS (ESI, m/e) [M+H]⁺ 613.2.

### Example 129: (S)-1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol

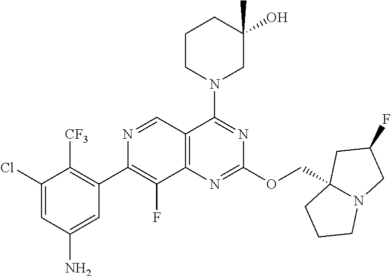 [SMILES: C[C@]1(O)CCCN(c2nc(OC[C@@]34CCCN3C[C@H](F)C4)nc3c(F)c(-c4cc(N)cc(Cl)c4C(F)(F)F)ncc23)C1]

Example 129 was prepared by similar procedure as described in Example 110 by replacing cyclopropanamine with (S)-3-methyl-piperidin-3-ol to give the title product. ¹H NMR (500 MHz, CD₃OD) δ 9.18 (s, 1H), 6.91 (s, 1H), 6.50 (s, 1H), 5.57-5.38 (m, 1H), 4.61-4.50 (m, 2H), 4.48-4.40 (m, 1H), 4.33-4.22 (m, 1H), 3.75-3.53 (m, 4H), 3.46-3.38 (m, 1H), 3.29-3.22 (m, 1H), 2.59-2.38 (m, 2H), 2.34-2.26 (m, 1H), 2.24-1.99 (m, 4H), 1.89-1.73 (m, 3H), 1.28 (s, 3H). MS (ESI, m/e) [M+H]⁺ 613.2.

### Example 130: 1-(7-(5-amino-3-chloro-2-(trifluoromethyl) phenyl)-8-fluoro-2-(((2R,7aS)-2-fluorotetrahydro-1H-pyrrolizin-7a(5H)-yl) methoxy) pyrido[4,3-d]pyrimidin-4-yl)-3-ethylpiperidin-3-ol

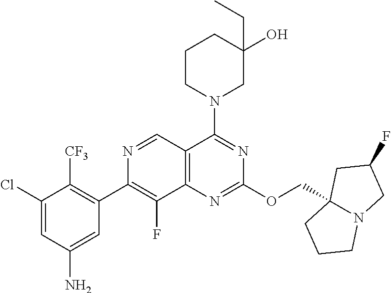 [SMILES: CCC1(O)CCCN(c2nc(OC[C@@]34CCCN3C[C@H](F)C4)nc3c(F)c(-c4cc(N)cc(Cl)c4C(F)(F)F)ncc23)C1]

Example 130 was prepared by similar procedure as described in Example 110 by replacing cyclopropanamine with 3-ethylpiperidin-3-ol to give the title product. ¹H NMR (500 MHz, CD₃OD) δ 9.20 (s, 1H), 6.91 (s, 1H), 6.50 (s, 1H), 5.51-5.34 (m, 1H), 4.64-4.28 (m, 4H), 3.69-3.39 (m, 5H), 3.26-3.18 (m, 1H), 2.53-1.50 (m, 12H), 1.02-0.94 (m, 3H). MS (ESI, m/e) [M+H]⁺ 627.4.

### Example 131: 3-(7-(5-amino-3-chloro-2-(trifluoromethyl) phenyl)-8-fluoro-2-(((2R,7aS)-2-fluorotetrahydro-1H-pyrrolizin-7a(5H)-yl) methoxy) pyrido[4,3-d]pyrimidin-4-yl)-3-azabicyclo[4.1.0]heptan-1-ol

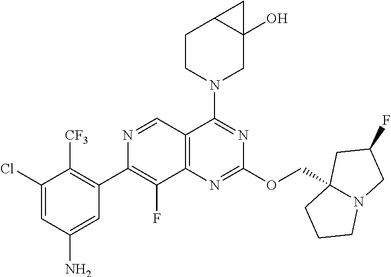 [SMILES: Nc1cc(Cl)c(C(F)(F)F)c(-c2ncc3c(N4CCC5CC5(O)C4)nc(OC[C@@]45CCCN4C[C@H](F)C5)nc3c2F)c1]

Example 131 was prepared by similar procedure as described in Example 110 by replacing cyclopropanamine with 3-azabicyclo[4.1.0]heptan-1-ol to give the title product. ¹H NMR (500 MHz, CD₃OD) δ 9.06 (s, 1H), 6.91 (s, 1H), 6.50 (s, 1H), 5.53-5.36 (m, 1H), 4.54-4.43 (m, 3H), 4.20-4.17 (m, 1H), 4.06-3.97 (m, 1H), 3.73-3.52 (m, 4H), 3.29-3.21 (m, 1H), 2.57-1.82 (m, 8H), 1.47-1.38 (m, 1H), 1.03-0.97 (m, 1H), 0.64-0.58 (m, 1H). MS (ESI, m/e) [M+H]⁺ 611.5.

### Assays

### Biochemical Assays

### KRAS WT and KRAS G12V Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-169) and GST-tagged GDP-loaded KRAS G12V (amino acids 1-169) were expressed in E. coli and purified in house. All protein and reaction solutions were prepared in assay buffer containing 50 mM HEPES pH7.5, 50 mM NaCl, 1 mM MgCl₂, 1 mM TCEP, 0.01% BSA, and 0.008% Brij-35. Purified WT KRAS (3 nM final concentration) or KRAS G12V protein (2 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (2.5 nM final assay concentration) for KRAS G12V was added to the assay plate, respectively. After 1 hr incubation at 24° C., Mab Anti-GST-Tb cryptate (Cisbio) and Streptavidin-XL665 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

### KRAS WT and KRAS G12D Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-188) and GST-tagged GDP-loaded KRAS G12D (amin acids 1-188) were expressed in E. coli and purified in house. All protein and reaction solutions were prepared in assay buffer containing 50 mM HEPES pH7.5, 50 mM NaCl, 1 mM MgCl₂, 1 mM TCEP, 0.01% BSA, and 0.008% Brij-35. Purified WT KRAS (3 nM final concentration) or KRAS G12D protein (0.5 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (4 nM final assay concentration) for KRAS G12D was added to the assay plate, respectively. After 1 hr incubation at 24° C., Mab Anti-GST-Tb cryptate (Cisbio) and Streptavidin-XL665 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

KRAS G12V pERK Assay

SW620 cell line was used in this study. Cells were maintained in RPMI 1640 supplemented with 10% fetal bovine serum (Thermo Fisher), 50 units/mL penicillin and streptomycin (Thermo Fisher) and kept at 37° C. in a humidified atmosphere of 5% CO2 in air. Cells were reinstated from frozen stocks that were laid down within 30 passages from the original cells purchased. 40000 cells per well were seeded into a 96-well plate and incubated overnight. Cells were treated with a 10-point dilution series. The final compound concentration is from 0 to 10 M. After 2 hrs compound treatment, cells were lysed, and the pERK1/2(THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+− cryptate (donor) labeled anti-phospho-ERK1/2 and 2 μL of D2 (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC50 determination was performed by fitting the curve of percent inhibition versus the log of the inhibitor concentration using Dotmatics.

KRAS G12D pERK Assay

AsPC-1 cell line was used in this study. Cells were maintained in RPMI-1640 supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. 30000 cells per well were seeded into a 96-well plate and incubated overnight. Cells were treated with a 10-point dilution series. The final compound concentration is from 0 to 10 μM. After 2 h compound treatment, cells were lysed, and the pERK1/2(THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+− cryptate (donor) labeled anti-phospho-ERK1/2 and 2 μL of D2 (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

### Activity Tables

Each of the compounds in Tables 1, 2 and 3 was tested in one or more of the biochemical assays provided herein and was found to have activity therein.

TABLE 1

| | G12V | G12D | SW620 | AsPC-1 |
|---|---|---|---|---|
| Compd | Probe | Probe | pERK | pERK |
| No. | IC₅₀/nM | IC₅₀/nM | IC50/Nm | IC50/nM |
| 1 | 4.2 | 22 | 129 | 2464 |
| 2 | 16 | | | |
| 3 | 6.2 | | | |
| 4 | 29 | | | |
| 5 | 45 | | | |
| 6 | 54 | | | |
| 7 | 4.2 | | 220 | |
| 8 | 43 | | | |
| 9 | 2.5 | | | |
| 10 | 19 | | | |
| 11 | 4.4 | | | |
| 12 | 3.7 | | | |
| 13 | 20 | | | |
| 14 | 13 | | | |
| 15 | 17 | | | |
| 16 | 14 | | | |
| 17 | 43 | | | |
| 18 | 13 | | | |
| 19 | 9.9 | | | |
| 20 | 7.9 | | | |
| 21 | 4.1 | | | |
| 22 | 367 | | | |
| 23 | 23 | | | |
| 24 | 24 | | | |
| 25 | 38 | | | |
| 26 | 11 | | | |
| 27 | 503 | | | |
| 28 | 3.8 | | | |
| 29 | 15 | | | |
| 30 | 3.5 | | | |
| 31 | 57 | | | |
| 32 | 21 | | | |

TABLE 2

| | G12V | G12D | SW620 | AsPC-1 |
|---|---|---|---|---|
| Compd | Probe | Probe | pERK | pERK |
| No. | IC₅₀/nM | IC₅₀/nM | IC50/Nm | IC50/nM |
| 33 | 2.6 | | 333 | |
| 34 | 52 | | | |
| 35 | 30 | | | |
| 36 | 19 | | | |
| 37 | 6.0 | | 449 | |
| 38 | 25 | | | |
| 39 | 5.5 | | 410 | |
| 40 | 12 | | | |
| 41 | 12 | | | |
| 42 | 70 | | | |
| 43 | 7.1 | | 356 | |
| 44 | 14 | | | |
| 45 | 15 | | | |
| 46 | 7.1 | | | |
| 47 | 1.9 | | | |
| 48 | 4.1 | | | |
| 49 | 3.0 | | | |
| 50 | 13 | | | |
| 51 | 11 | | | |
| 52 | 19 | | | |
| 53 | 12 | | | |
| 54 | 41 | | | |
| 55 | 168 | | | |
| 56 | 22 | | | |
| 57 | 180 | | | |
| 58 | 40 | | | |
| 59 | 27 | | | |
| 60 | 2.1 | | 413 | |
| 61 | 14 | | | |
| 62 | 22 | | | |
| 63 | 11 | | | |
| 64 | 4.3 | | | |
| 65 | 4.1 | | | |
| 66 | 7.7 | | | |
| 67 | 30 | | | |
| 68 | 0.77 | 6.6 | 76 | 686 |
| 69 | 17 | | | |
| 70 | 7.5 | | | |
| 71 | 10 | | | |
| 72 | 2.0 | | | |
| 73 | 6.8 | | | |
| 74 | 1.9 | | | |
| 75 | 3.8 | | | |
| 76 | 21 | | | |
| 77 | 17 | | | |
| 78 | 3.3 | | | |
| 79 | 28 | | | |
| 80 | 17 | | | |
| 81 | 4.9 | | | |
| 82 | 0.74 | | 199 | |
| 83 | 6.4 | | | |
| 84 | 0.98 | | | |
| 85 | 2.3 | | | |
| 86 | 0.8 | 23 | 715 | 5440 |
| 87 | 0.77 | | 108 | |
| 88 | 1.4 | | | |
| 89 | 2.4 | | | |
| 90 | 4.2 | | | |
| 91 | 3.8 | | | |
| 92 | 2.8 | | | |
| 93 | 2.2 | | | |
| 94 | 2.5 | | | |
| 95 | 2.7 | | | |
| 96 | 1.5 | | | |
| 97 | 3.8 | | | |
| 98 | 51 | | | |
| 99 | 2.8 | | | |
| 100 | 0.74 | 0.34 | 18 | 56 |
| 101 | 0.28 | | 11 | |
| 102 | 46 | | | |
| 103 | 11 | | | |
| 104 | 7.0 | | | |
| 105 | 25 | | | |
| 106 | 23 | | | |
| 107 | 73 | | | |
| 108 | >1000 | | | |
| 109 | >1000 | | | |

TABLE 3

| | G12V | G12D | SW620 | AsPC-1 |
|---|---|---|---|---|
| Compd | Probe | Probe | pERK | pERK |
| No. | IC₅₀/nM | IC₅₀/nM | IC50/Nm | IC50/nM |
| 110 | 17 | | | |
| 111 | 40 | | | |
| 112 | 19 | | | |
| 113 | 11 | | | |
| 114 | 9.2 | | | |
| 115 | 6.5 | | | |
| 116 | 4.4 | | | |
| 117 | 12 | | | |
| 118 | 23 | | | |
| 119 | 24 | | | |
| 120 | 6.6 | | | |
| 121 | 13 | | | |
| 122 | 28 | | | |
| 123 | 22 | | | |
| 124 | 15 | | | |
| 125 | 81 | | | |
| 126 | 6.3 | | | |
| 127 | 21 | | | |
| 128 | 2.6 | 1.2 | 84 | 76 |
| 129 | 15 | | | |
| 130 | 32 | | | |
| 131 | 23 | | | |

As demonstrated by the data in Tables 1-3, the inventors surprisingly and unexpectedly discovered that the exemplary compounds in Tables 1-3 modulate or inhibit the activity of KRAS G12V.

A number of references have been cited, the disclosures of which are incorporated herein by reference in their entirety.

1. A compound of formula (I): 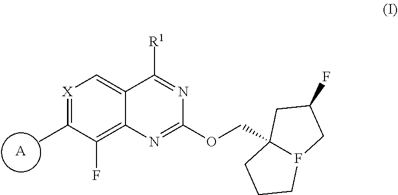
or a pharmaceutically acceptable salt, tautomer, stereoisomer, or enantiomer thereof,
wherein:
ring A is 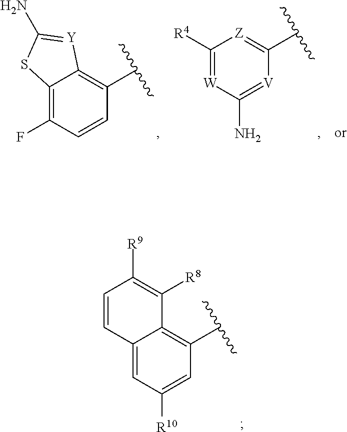
X is N, or C—R²;
Y is N, or C—CN;
Z is N, or C—R³;
W is N, or C—R⁵;
V is N, or C—R⁷;
R¹ is —NR^{1a}R^{1b};
R² is halogen, or unsubstituted or substituted alkyl;
R³ is halogen, or unsubstituted or substituted alkyl;
R⁴ is halogen, —NO₂, or unsubstituted or substituted alkyl;
R⁵ is hydrogen, halogen, or —CN;
R⁷ is hydrogen, halogen, or —CN;
each of R⁸, R⁹, and R¹⁰ is independently hydrogen, halogen, unsubstituted or substituted alkyl, or —OH;
R^{1a} and R^{1b} are each independently hydrogen, unsubstituted or substituted alkyl, unsubstituted or substituted cycloalkyl, unsubstituted or substituted cycloalkylalkyl, unsubstituted or substituted heterocyclyl, unsubstituted or substituted heterocyclylalkyl, provided that R^{1a} and R^{1b} are not both hydrogen; or
R^{1a} and R^{1b}, together with the nitrogen atom to which they are attached to, form an unsubstituted or substituted heterocyclyl.

2. The compound of claim 1,
wherein ring A is

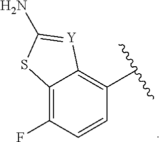

3. The compound of claim 2, wherein Y is N.

4. The compound of claim 2, wherein Y is C—CN.

5. The compound of claim 2, wherein X is C—R².

6. The compound of claim 2, wherein R² is halogen, or alkyl which is unsubstituted or substituted by halogen; preferably Cl, or CF₃; more preferably CF₃.

7. The compound of claim 2, wherein R¹ is —NHR^{1a}.

8. The compound of claim 7, wherein R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl.

9. The compound of claim 1,
wherein ring A is

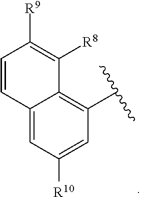

10. The compound of claim 9, wherein X is C—R².

11. The compound of claim 2, wherein R^{1a} and R^{1b}, together with the nitrogen atom to which they are attached to, form an unsubstituted or substituted monocyclic heterocyclyl or an unsubstituted or substituted spiro heterocyclyl, said unsubstituted or substituted monocyclic heterocyclyl or the unsubstituted or substituted spiro heterocyclyl comprises zero or one or two additional heteroatoms selected from oxygen, nitrogen, or optionally oxidized sulfur, preferably R¹ is unsubstituted or substituted heterocyclyl selected from unsubstituted or substituted azetidinyl, unsubstituted or substituted pyrrolidyl, unsubstituted or substituted piperidyl, unsubstituted or substituted azepanyl, unsubstituted or substituted 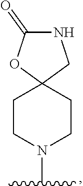 [SMILES: *N1CCC2(CC1)CNC(=O)O2]
unsubstituted or substituted 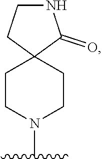 [SMILES: *N1CCC2(CCNC2=O)CC1]
unsubstituted or substituted 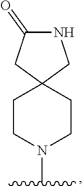 [SMILES: *N1CCC2(CC1)CNC(=O)C2]
unsubstituted or substituted 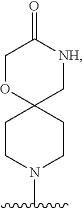 [SMILES: *N1CCC2(CC1)CNC(=O)CO2]
or unsubstituted or substituted

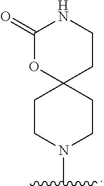 [SMILES: *N1CCC2(CCNC(=O)O2)CC1]

12. The compound of claim 1,
wherein ring A is

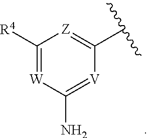

13. The compound of claim 12, wherein X is C—R².

14. The compound of claim 13, wherein R² is halogen, or alkyl which is unsubstituted or substituted by halogen; preferably Cl, or CF₃; more preferably Cl.

15. The compound of claim 14, wherein ring A is

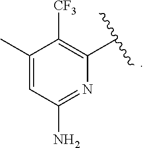 [SMILES: *c1nc(N)cc(C)c1C(F)(F)F]

16. The compound of claim 12, wherein R^{1a} and R^{1b}, together with the nitrogen atom to which they are attached to, form an unsubstituted or substituted monocyclic heterocyclyl or an unsubstituted or substituted spiro heterocyclyl, said unsubstituted or substituted monocyclic heterocyclyl or the unsubstituted or substituted spiro heterocyclyl comprises zero, one, or two additional heteroatoms selected from oxygen, nitrogen, or optionally oxidized sulfur; preferably R¹ is unsubstituted or substituted heterocyclyl selected from unsubstituted or substituted azetidinyl, unsubstituted or substituted pyrrolidyl, unsubstituted or substituted piperidyl, unsubstituted or substituted morpholinyl, or unsubstituted or substituted

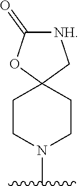 [SMILES: *N1CCC2(CC1)CNC(=O)O2]

17. The compound of claim 12, wherein R¹ is

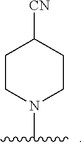 [SMILES: *N1CCC(C#N)CC1]

18. The compound of claim 17,
wherein ring A is

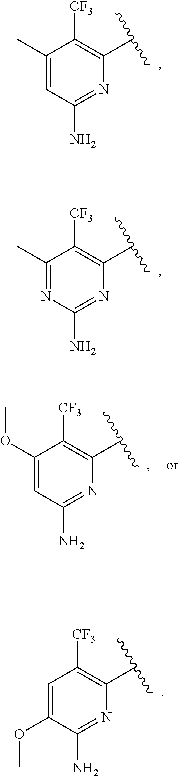 [SMILES: *c1nc(N)cc(C)c1C(F)(F)F ; *c1nc(N)nc(C)c1C(F)(F)F ; *c1nc(N)cc(OC)c1C(F)(F)F ; *c1nc(N)c(OC)cc1C(F)(F)F]

19. The compound of claim 12, wherein X is N.

20. The compound of claim 19, wherein ring A is

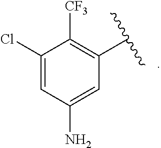 [SMILES: *c1cc(N)cc(Cl)c1C(F)(F)F]

21. The compound of claim 19, wherein R¹ is —NHR^{1a}.

22. The compound of claim 21, wherein R^{1a} is unsubstituted or substituted cycloalkyl, or unsubstituted or substituted heterocyclyl, wherein at least one ring of R^{1a} is cyclopropyl, cyclobutyl, cyclopentyl, or cyclohexyl; preferably cyclopropyl.

23. The compound of claim 19, wherein R^{1a} and R^{1b}, together with the nitrogen atom to which they are attached to, form an unsubstituted or substituted monocyclic heterocyclyl or an unsubstituted or substituted spiro heterocyclyl, said unsubstituted or substituted monocyclic heterocyclyl or the unsubstituted or substituted spiro heterocyclyl comprises zero or one or two additional heteroatoms selected from oxygen, nitrogen, or optionally oxidized sulfur, preferably at least one ring of R¹ is piperidyl, or morpholinyl.

24. The compound of claim 23, wherein R¹ is optionally substituted by one or more C₁₋₄ alkyl, halogen, —CN, or —OH, wherein each of the C₁₋₄ alkyl is independently optionally substituted by one or more halogen, —CN, or —OH.

25. The compound of claim 19, wherein R¹ is

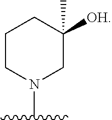 [SMILES: *N1CCC[C@@](C)(O)C1]

26. The compound of claim 25, wherein ring A is

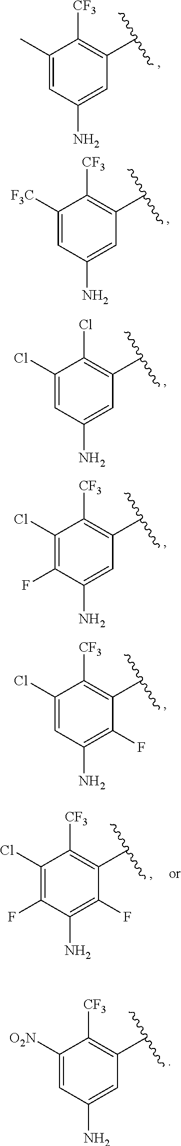

27. The compound of claim 1, wherein R^{1a} and R^{1b}, together with the nitrogen atom to which they are attached to, form unsubstituted or substituted monocyclic heterocyclyl, unsubstituted or substituted bicyclic heterocyclyl, unsubstituted or substituted tricyclic heterocyclyl, or unsubstituted or substituted spirocyclic heterocyclyl.

28. The compound of claim 1, wherein R¹ is not unsubstituted or substituted piperazinyl.

29. The compound of claim 1, wherein R¹ is not unsubstituted or substituted 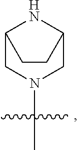 [SMILES: *N1CC2CCC(C1)N2]
unsubstituted or substituted 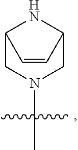 [SMILES: *N1CC2C=CC(C1)N2]
unsubstituted or substituted 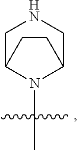 [SMILES: *N1C2CCC1CNC2]
unsubstituted or substituted 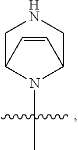 [SMILES: *N1C2C=CC1CNC2]
unsubstituted or substituted 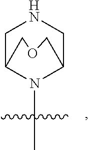 [SMILES: *N1C2CNCC1COC2]
, unsubstituted or substituted 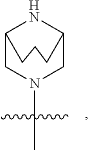 [SMILES: *N1CCNC(CCCC)C1]
or unsubstituted or substituted

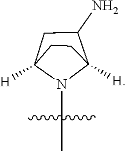

30. The compound of claim 1, wherein R¹ is not unsubstituted or substituted 3,8-diazabicyclo[3.2.1]octan-3-yl, or unsubstituted or substituted

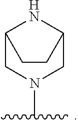 [SMILES: *N1CC2CCC(C1)N2]

31. The compound of claim 1, wherein the compound is selected from Table 1, Table 2 and Table 3.

32. A pharmaceutical composition comprising an effective amount of a compound of claim 1, or a pharmaceutically acceptable salt, tautomer, isotopologue, stereoisomer, or prodrug thereof, and a pharmaceutically acceptable carrier, excipient or vehicle.

33. A method for inhibiting the activity of KRAS mutant protein in a cell, comprising contacting said cell with an effective amount of a compound of claim 1, or a pharmaceutically acceptable salt, tautomer, isotopologue, stereoisomer, or prodrug thereof, optionally wherein the KRAS mutant protein is KRAS G12D and/or G12V mutant protein.

34. A method for treatment or prevention of cancer, the method comprising administering to a subject in need thereof an effective amount of a compound of claim 1, or a pharmaceutically acceptable salt, tautomer, isotopologue, stereoisomer, or prodrug thereof, optionally wherein the cancer is mediated by KRAS mutation; preferably KRAS G12D and/or G12V mutation.
